(19) United States
(12) Patent Application Publication
(10) Pub. No.: US 2015/0018235 A1
(43) Pub. Date: Jan. 15, 2015

(54) Methods and Compositions for the Treatment and Diagnosis of Pancreatic Cancer

(71) Applicant: Oncocyte Corporation, Alameda, CA (US)

(72) Inventors: Karen Chapman, Mill Valley, CA (US); Joseph Wagner, San Ramon, CA (US); Michael West, Mill Valley, CA (US)

(21) Appl. No.: 14/371,234

(22) PCT Filed: Jan. 14, 2013
(86) PCT No.: PCT/US13/21462

§ 371 Date: Jul. 9, 2014

Related U.S. Application Data
(60) Provisional application No. 61/586,601, filed on Jan. 13, 2012.

(51) Int. Cl. G01N 33/574; C12Q 1/68

(52) U.S. Cl. CPC G01N 33/57484; C12Q 1/6886; C12Q 2600/112
USPC 506/9; 506/16; 506/18; 506/14

(57) ABSTRACT

The invention provides methods, compositions and kits for the detection and treatment of pancreatic cancer.

This application claims priority to U.S. Provisional Application No. 61/586,601 filed on Jan. 13, 2012 the entire contents of which is hereby incorporated by reference.

### FIELD OF THE INVENTION

The field of the invention relates to cancer and the diagnosis and treatment of cancer.

### BACKGROUND

Early detection of cancer can impact treatment outcomes and disease progression. Typically, cancer detection relies on diagnostic information obtained from biopsy, x-rays, CAT scans, NMR and the like. These procedures may be invasive, time consuming and expensive. Moreover, they have limitations with regard to sensitivity and specificity. There is a need in the field of cancer diagnostics for a highly specific, highly sensitive, rapid, inexpensive, and relatively non-invasive method of diagnosing cancer. Various embodiments of the invention described below meet this need as well as other needs existing in the field of diagnosing and treating cancer.

### SUMMARY OF THE INVENTION

Embodiments of the disclosure provide methods of diagnosis, prognosis and treatment of cancer, e.g. pancreatic cancer. Other embodiments provide compositions relating to the diagnosis, prognosis and treatment of cancer, such as pancreatic cancer.

In certain embodiments the invention provides a method of detecting pancreatic cancer in a subject comprising a) obtaining a sample from a subject; b) contacting the sample obtained from the subject with one or more agents that detect one or more markers expressed by a pancreatic cancer cell c) contacting a non-cancerous cell with the one or more agents from b); and d) comparing the expression level of the marker in the sample obtained from the subject with the expression level in the non-cancerous cell, wherein a higher level of expression of the marker in the sample compared to the non-cancerous cell indicates that the subject has pancreatic cancer. Suitable markers include the genes encoded for by any of the genes disclosed infra.

In some embodiments the invention provides a method of detecting pancreatic cancer in a subject comprising a) obtaining a sample from a subject b) contacting the sample obtained from the subject with one or more agents that detect expression of one or more of the markers encoded by genes chosen from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof; c) contacting a non-cancerous cell with the one or more agents from b); and d) comparing the expression level of one or more of the markers encoded by genes chosen from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof in the sample with the expression level in the non-cancerous cell, wherein a higher level of expression in the sample of one or more of the markers encoded by genes chosen from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof in the sample obtained from the subject compared to the non-cancerous cell indicates that the subject has pancreatic cancer.

In other embodiments the invention provides a method of detecting pancreatic cancer in a subject comprising a) obtaining a sample from a subject b) contacting the sample obtained from the subject with one or more agents that detect expression of a panel of markers encoded by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof; c) contacting a non-cancerous cell, with the one or more agents from b); and d) comparing the expression level of the panel of markers encoded for by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof in the sample obtained from the subject with the expression level of the panel of markers encoded for by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof, in the non-cancerous cell, wherein a higher level of expression of the panel of markers encoded for by genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof in the sample compared to the non-cancerous cell indicates that the subject has pancreatic cancer.

In other embodiments the invention provides a method of detecting pancreatic adenocarcinoma in a subject comprising a) obtaining a sample from a subject b) contacting the sample obtained from the subject with one or more agents that detect expression of a panel of markers encoded by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, or a complement thereof; c) contacting a non-cancerous cell, with the one or more agents from b); and d) comparing the expression level of the panel of markers encoded for by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, or a complement thereof in the sample obtained from the subject with the expression level of the panel of markers encoded for by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, or a complement thereof, in the non-cancerous cell, wherein a higher level of expression of the panel of markers encoded for by genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1 or a complement thereof in the sample compared to the non-cancerous cell indicates that the subject has pancreatic adenocarcinoma.

In other embodiments the invention provides a method of detecting pancreatic neuroendocrine carcinoma in a subject comprising a) obtaining a sample from a subject b) contacting the sample obtained from the subject with one or more agents that detect expression of a panel of markers encoded by the genes WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, or a complement thereof; c) contacting a non-cancerous cell, with the one or more agents from b); and d) comparing the expression level of the panel of markers encoded for by the genes WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, or a complement thereof in the sample obtained from the subject with the expression level of the panel of markers encoded for by the genes WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, or a complement thereof, in the non-cancerous cell, wherein a higher level of expression of the panel of markers encoded for by genes WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP or a complement thereof in the sample compared to the non-cancerous cell indicates that the subject has pancreatic neuroendocrine carcinoma.

In further embodiments the invention provides a method of detecting pancreatic cancer cells in a sample comprising a) obtaining a sample b) contacting the sample obtained in a) with one or more agents that detect expression of one or more of the markers encoded by genes chosen from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6 orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof; c) contacting a non-cancerous cell with the one or more agents from b); and d) comparing the expression level of one or more of the markers encoded by genes chosen from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof in the sample obtained in a) with the expression level of one or more of the markers encoded by genes chosen from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof, in the non-cancerous cell, wherein a higher level of expression of one or more of the markers encoded by genes chosen from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6 orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof, in the sample compared to the non-cancerous cell indicates that the sample contains pancreatic cancer cells. The sample may be an in vitro sample or an in vivo sample, or derived from an in vivo sample.

With regard to the embodiments described in the preceding paragraphs, the sample may be any sample as described infra, for example, a bodily fluid, such as blood, serum or urine. The sample may be a cellular sample or the extract of a cellular sample. The sample may be a tissue sample. Nucleic acids and/or proteins may be isolated from the sample. Nucleic acids such as RNA may be transcribed into cDNA. The agent may be one or more molecules that bind specifically to one or more proteins expressed by the cancer cell or one or more nucleic acids expressed by the cell. For example, the agent may be a protein such as an antibody that binds specifically to the protein expressed by one of the marker genes identified infra. The agent may be one or more nucleic acids that hybridize to a nucleic acid expressed by the cancer cell. The nucleic acid expressed by the cancer cell may be an RNA molecule, e.g. an mRNA molecule. The nucleic acid molecule that hybridizes to the nucleic acid expressed by the cancer cell may be a DNA molecule, such as a DNA probe.

In still other embodiments the invention provides a composition of matter useful in distinguishing a pancreatic cancer cell from a non-cancerous cell comprising one or more molecules that specifically bind to a molecule expressed at higher levels by a pancreatic cancer cell compared to a non-cancer cell. As an example, the composition may comprise a protein, that binds to one or more molecules expressed by the pancreatic cancer cell at higher levels compared to the non-cancer cell. As another example, the composition may comprise a nucleic acid that binds to one or more molecules expressed by the pancreatic cancer cell at higher levels compared to the non-cancer cell.

In some embodiments the invention provides a composition of matter comprising one or more proteins, such as an antibody, that specifically binds to a molecule expressed by a pancreatic cancer cell chosen from the markers encoded by the genes listed in tables 1, 2, 6 or fragments thereof. The molecule expressed by the pancreatic cancer cell may be expressed by the cancer cell at a level that is higher than the level expressed by a non-cancerous cell.

In some embodiments the invention provides a composition of matter comprising one or more proteins, such as an antibody, that specifically binds to a molecule expressed by a pancreatic cancer cell chosen from the markers encoded by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2. The molecule expressed by the pancreatic cancer cell may be expressed by the cancer cell at a level that is higher than the level of the same marker expressed by a non-cancerous cell.

In further embodiments the invention provides a composition of matter comprising a plurality of proteins, such as a plurality antibodies, that specifically binds to a panel of molecules expressed by a pancreatic cancer cell wherein the panel of markers comprises molecule encoded by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof. The panel of markers may be expressed at a level that is higher in the cancer cell than the level of the panel of markers in a non-cancerous cell.

In further embodiments the invention provides a composition of matter comprising a plurality of proteins, such as a plurality antibodies, that specifically binds to a panel of molecules expressed by a pancreatic adenocarcinoma cell wherein the panel of markers comprises molecule encoded by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, or a complement thereof. The panel of markers may be expressed in the adenocarcinoma cell at a level that is higher than the level of the panel of markers in a non-cancerous cell.

In other embodiments the invention provides a composition of matter comprising a plurality of proteins, such as a plurality antibodies, that specifically binds to a panel of molecules expressed by a pancreatic neuroendocrine carcinoma cell wherein the panel of markers comprises molecule encoded by the genes WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, or a complement thereof. The panel of markers may be expressed in the neuroendocrine carcinoma cell at a level that is higher than the level of the panel of markers in a non-cancerous cell.

In certain embodiments the invention provides a composition of matter comprising a protein, such as an antibody, that specifically binds to a molecule expressed by an pancreatic cancer cell chosen from a molecule encoded by one or more of the genes chosen from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof. The molecule expressed by the pancreatic cancer cell may be expressed by the pancreatic cancer cell at level that is higher than the level expressed by a non-cancerous cell.

In other embodiments the invention provides a composition of matter comprising a nucleic acid that specifically binds to a molecule, such as an mRNA molecule, expressed by a pancreatic cancer cell wherein the molecule is chosen from a marker encoded for by the genes listed in tables 1, 2, 6 or fragments thereof. The molecule expressed by the pancreatic cancer cell may be expressed by the pancreatic cancer cell at level that is higher than the level expressed by a non-cancerous cell.

In other embodiments the invention provides a composition of matter comprising a nucleic acid that specifically binds to a molecule, such as an mRNA molecule, expressed by a pancreatic cancer cell wherein the molecule is chosen from a marker encoded for by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2. The molecule expressed by the pancreatic cancer cell may be expressed by the cancer cell at level that is higher than the level expressed by a non-cancerous cell.

In still further embodiments the invention provides a method of determining if a pancreatic cancer in a subject is advancing comprising a) measuring the expression level of one or more markers associated with pancreatic cancer at a first time point; b) measuring the expression level of the one or more markers measured in a) at a second time point, wherein the second time point is subsequent to the first time point; and c) comparing the expression level measured in a) and b), wherein an increase in the expression level of the one or more markers in b) compared to a) indicates that the subject's pancreatic cancer is advancing. Suitable markers include those markers encoded for by the genes listed in tables 1, 2, 6 or fragments thereof.

In some embodiments the invention provides a method of determining if a pancreatic cancer in a subject is advancing comprising obtaining a sample and a) measuring the expression level of the panel of markers encoded for by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, at a first time point; b) measuring the expression level of the markers measured in a) at a second time point, wherein the second time point is subsequent to the first time point; and c) comparing the expression level measured in a) and b), wherein an increase in the expression level of the markers at the second time point compared to the first time point indicates that the subject's pancreatic cancer is advancing.

In some embodiments the invention provides antigens (i.e. cancer-associated polypeptides) associated with pancreatic cancer as targets for diagnostic and/or therapeutic antibodies. In some embodiments, the antigen may be chosen from a protein encoded by one or more genes listed in tables 1, 2, 6 or fragments thereof, or a combination of proteins encoded by a gene listed in tables 1, 2, 6 or fragments thereof.

In some embodiments the invention provides antigens (i.e. cancer-associated polypeptides) associated with pancreatic cancer as targets for diagnostic and/or therapeutic antibodies. In some embodiments, the antigen may include a panel of proteins encoded by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a fragment thereof.

In yet other embodiments the invention provides a method of eliciting an immune response to a pancreatic cancer cell comprising contacting a subject with a protein or protein fragment that is expressed by a pancreatic cancer cell thereby eliciting an immune response to the pancreatic cancer cell. As an example the subject may be contacted intravenously or intramuscularly with protein or protein fragment.

In further embodiments the invention provides a method of eliciting an immune response to a pancreatic cancer cell comprising contacting a subject with one or more proteins or protein fragments that is encoded by a gene chosen from the genes listed in tables 1, 2, 6 or fragments thereof, thereby eliciting an immune response to a pancreatic cancer cell. As an example the subject may be contacted with the protein or the protein fragment intravenously or intramuscularly.

In yet other embodiments the invention provides a kit for detecting pancreatic cancer cells in a sample. The kit may comprise one or more agents that detect expression of any the cancer associated sequences disclosed infra e.g. those provided in Tables 1, 2, and 6. The agents may bind to one or more of the cancer associated sequences disclosed infra. The kit may include agents that are proteins and/or nucleic acids for example. In one embodiment the kit provides a plurality of agents. The agents may be able to detect the panel of markers encoded by the genes comprising PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof. In certain embodiments the agent binds specifically to a gene product of one or more of the genes disclosed infra, e.g. one or more the genes disclosed in Tables 1, 2, and 6. The gene product may be a protein or a peptide or a nucleic acid such as an mRNA or a combination thereof.

In yet other embodiments the invention provides a kit for detecting pancreatic adenocarcinoma cancer cells in a sample. The kit may comprise one or more agents that detect expression of any the cancer associated sequences disclosed infra e.g. those provided in Tables 1, 2, and 6. The agents may bind to one or more of the cancer associated sequences disclosed infra. The kit may include agents that are proteins and/or nucleic acids for example. In one embodiment the kit provides a plurality of agents. The agents may be able to detect the panel of markers encoded by the genes comprising PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, or a complement thereof. In certain embodiments the agent binds specifically to a gene product of one or more of the genes disclosed infra, e.g. one or more the genes chosen from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1. The gene product may be a protein or a peptide or a nucleic acid such as an mRNA or a combination thereof.

In yet other embodiments the invention provides a kit for detecting pancreatic neuroendocrine carcinoma cells in a sample. The kit may comprise one or more agents that detect expression of any the cancer associated sequences disclosed infra e.g. those provided in Tables 1, 2, and 6. The agents may bind to one or more of the cancer associated sequences disclosed infra. The kit may include agents that are proteins and/or nucleic acids for example. In one embodiment the kit provides a plurality of agents. The agents may be able to detect the panel of markers encoded by the genes comprising WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, WP, or a complement thereof. In certain embodiments the agent binds specifically to a gene product of one or more of the genes chosen from The gene product may be a protein or a peptide or a nucleic acid such as an mRNA or a combination thereof.

In yet other embodiments the invention provides a kit for detecting pancreatic cancer cells in a sample. The kit may comprise one or more agents that detect expression of any the cancer associated sequences disclosed infra e.g. those provided in Tables 1, 2, and 6. The agents may bind to one or more of the cancer associated sequences disclosed infra. The kit may include agents that are proteins and/or nucleic acids for example. In one embodiment the kit provides a plurality of agents. The agents may be able to detect the panel of markers encoded by the genes comprising UBD, LAMC2, PPY, REG4, REG1b, LCN2, MMP11, COL10A and MMP7, or a complement thereof. The agents may bind to a gene product encoded for by one or more genes chosen from UBD, LAMC2, PPY, REG4, REG1b, LCN2, MMP11, COL10A and MMP7. The gene product may be a protein or a peptide or a nucleic acid such as an mRNA or a combination thereof.

In yet other embodiments the invention provides a kit for detecting pancreatic cancer cells in a sample. The kit may comprise one or more agents that detect expression of any the cancer associated sequences disclosed infra. The kit may include agents that are proteins and/or nucleic acids for example. In one embodiment the kit provides a plurality of agents. The agents may be able to detect the panel of markers encoded by the genes comprising PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof. In certain embodiments the agent binds specifically to a gene product of one or more of the genes disclosed infra, e.g. one or more the genes disclosed in Tables 1, 2, and 6. The gene product may be a protein or a peptide or a nucleic acid such as an mRNA or a combination thereof.

In still other embodiments the invention provides a kit for detecting pancreatic cancer in a sample comprising a plurality of agents that specifically bind to a molecule encoded for by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2. The agent may be a nucleic acid such as a DNA molecule or a protein or a peptide. The molecule encoded for by the gene may be a nucleic acid such as an mRNA or a protein or a peptide or a combination thereof.

In other embodiments the invention provides a kit for detection of pancreatic cancer in a sample obtained from a subject. The kit may comprise one or more agents that bind specifically to a molecule expressed specifically by a pancreatic cancer cell, e.g. one or more of the markers encoded for by the genes provided in Tables 1, 2, and 6. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The detectible substance may be linked the agent that specifically binds to a molecule expressed by a pancreatic cancer cell. The kit may further contain a positive control (e.g. one or more pancreatic cancer cells; or specific known quantities of the molecule expressed by the pancreatic cancer cell) and a negative control (e.g. a tissue or cell sample that is non-cancerous).

In some embodiments the invention provides a kit for the detection of pancreatic cancer comprising one or more agents that specifically bind one or more markers encoded by genes chosen from a gene disclosed infra., e.g., PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2. The agent may be a protein, such as an antibody. Alternatively, the agent may be a nucleic such as a DNA molecule or an RNA molecule. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The detectable substance may be linked to the agent that specifically binds the one or more markers disclosed infra. The kit may further contain a positive control (e.g. one or more pancreatic cancer cells; or specific known quantities of the molecule expressed by the pancreatic cancer cell) and a negative control (e.g. a tissue or cell sample that is non-cancerous). As an example the kit may take the form of an ELISA or a DNA microarray. In some embodiments the kit may include one or more antibodies suitable for use in a fluorescent activated cell sorter, e.g. use in flow cytometry.

Some embodiments are directed to a method of treating pancreatic cancer in a subject, the method comprising administering to a subject in need thereof a therapeutic agent modulating the activity of a pancreatic cancer associated protein, wherein the cancer associated protein is encoded by the genes listed in tables 1, 2, 6 or fragments thereof, homologs thereof, combinations thereof, or a fragment thereof. In some embodiments, the therapeutic agent binds to the cancer associated protein. In some embodiments, the therapeutic agent is an antibody. In some embodiments, the antibody may be a monoclonal antibody or a polyclonal antibody. In some embodiments, the antibody is a humanized or human antibody. In some embodiments the antibody may be conjugated with a drug or a toxin.

In some embodiments, a method of treating pancreatic cancer in a subject may comprise administering to a subject in need thereof a therapeutic agent that modulates the expression of one or more genes chosen from the genes listed in tables 1, 2, 6 or fragments thereof, fragments thereof, homologs thereof, and/or complements thereof.

In further embodiments, the invention provides a method of treating pancreatic cancer may comprise a gene knockdown of one or more of the genes listed in tables 1, 2, 6 or fragments thereof, homologs thereof, and or compliments thereof.

In still other embodiments, the present invention provides methods of screening a drug candidate for activity against pancreatic cancer, the method comprising: (a) contacting a cell that expresses one or more pancreatic cancer associated genes chosen from the genes listed in tables 1, 2, 6 or fragments thereof with a drug candidate; (b) detecting an effect of the drug candidate on expression of the one or more pancreatic cancer associated genes in the cell from a); and (c) comparing the level of expression of one or more of the genes recited in a) in the absence of the drug candidate to the level of expression of the one or more genes recited in a) in the presence of the drug candidate; wherein a decrease in the expression of the pancreatic cancer associated gene in the presence of the drug candidate indicates that the candidate has activity against pancreatic cancer.

In some embodiments, the present invention provides methods of visualizing a pancreatic cancer tumor comprising a) targeting one or more pancreatic cancer associated proteins with a labeled molecule that binds specifically to the cancer tumor, wherein the pancreatic cancer associated protein is selected from a protein encoded for by one or more genes chosen from those listed in the genes listed in tables 1, 2, 6 or fragments thereof; and b) detecting the labeled molecule, wherein the labeled molecule visualizes the tumor. Visualization may be done in vivo, or in vitro.

In yet other embodiments the invention provides methods of visualizing a pancreatic cancer tumor comprising a) targeting one or more pancreatic cancer associated genes, e.g. one or more genes encoded for by the genes listed in tables 1, 2, 6 or fragments thereof, with a labeled molecule, such as a nucleic acid that binds specifically to the cancer tumor genes chosen from the genes listed in tables 1, 2, 6 or fragments thereof; and b) detecting the labeled molecule, wherein the labeled molecule visualizes the tumor. Visualization may be done in vivo, or in vitro.

### DESCRIPTION OF DRAWINGS

For a fuller understanding of the nature and advantages of the present invention, reference should be had to the following detailed description taken in connection with the accompanying drawings, in which:

FIG. 1 shows the expression of PPY in normal cells and tissues versus pancreatic tumors.

FIG. 2 shows the expression of CHGB in normal cells and tissues versus pancreatic tumors.

FIG. 3 shows the expression of ACE2 in normal cells and tissues versus pancreatic tumors.

FIG. 4 shows the expression of COL10A1 in normal cells and tissues versus pancreatic tumors.

FIG. 5 shows the expression of PRSS7 in normal cells and tissues versus pancreatic tumors.

FIG. 6 shows the expression of MEP1B in normal cells and tissues versus pancreatic tumors.

FIG. 7 shows the expression of MMP12 in normal cells and tissues versus pancreatic tumors.

FIG. 8 shows the expression of MMP11 in normal cells and tissues versus pancreatic tumors.

FIG. 9 shows the expression of TM4SF20 in normal cells and tissues versus pancreatic tumors.

FIG. 10 shows the expression of AQP10 in normal cells and tissues versus pancreatic tumors.

FIG. 11 shows the expression of REG1P in normal cells and tissues versus pancreatic tumors.

FIG. 12 shows the expression of KCNIP1 in normal cells and tissues versus pancreatic tumors.

FIG. 13 shows the expression of UBD in normal cells and tissues versus pancreatic tumors.

FIG. 14 shows the expression of MS4A10 in normal cells and tissues versus pancreatic tumors.

FIG. 15 shows the expression of CXCL5 in normal cells and tissues versus pancreatic tumors.

FIG. 16 shows the expression of REG3G in normal cells and tissues versus pancreatic tumors.

FIG. 17 shows the expression of PTPRR in normal cells and tissues versus pancreatic tumors.

FIG. 18 shows the expression of CST1 in normal cells and tissues versus pancreatic tumors.

FIG. 19 shows the expression of MMP7 in normal cells and tissues versus pancreatic tumors.

FIG. 20 shows the expression of COMP in normal cells and tissues versus pancreatic tumors.

FIG. 21 shows the expression of WNT4 in normal cells and tissues versus pancreatic tumors.

FIG. 22 shows the expression of PCSK2 in normal cells and tissues versus pancreatic tumors.

FIG. 23 shows the expression of CHGB in normal cells and tissues versus pancreatic tumors.

FIG. 24 shows the expression of PCSK1 in normal cells and tissues versus pancreatic tumors.

FIG. 25 shows the expression of PCSK1N in normal cells and tissues versus pancreatic tumors.

FIG. 26 shows the expression of SCGN in normal cells and tissues versus pancreatic tumors.

FIG. 27 shows the expression of PPY in normal cells and tissues versus pancreatic tumors.

FIG. 28 shows the expression of SMOC1 in normal cells and tissues versus pancreatic tumors.

FIG. 29 shows the expression of VIP in normal cells and tissues versus pancreatic tumors.

FIG. 30 shows the expression of UBD in normal serum versus serum from patients with pancreatic cancer.

FIG. 31 shows the expression of LAMC2 in normal serum versus serum from patients with pancreatic cancer.

FIG. 32 shows the expression of PPY in normal serum versus serum from patients with pancreatic cancer.

FIG. 33 shows the expression of REG4 in normal serum versus serum from patients with pancreatic cancer.

FIG. 34 shows the expression of REG1b in normal serum versus serum from patients with pancreatic cancer.

FIG. 35 shows the expression of LCN2 in normal serum versus serum from patients with pancreatic cancer.

FIG. 36 shows the expression of MMP11 in normal serum versus serum from patients with pancreatic cancer.

FIG. 37 shows the expression of COL10A in normal serum versus serum from patients with pancreatic cancer.

FIG. 38 shows the expression of MMP7 in normal serum versus serum from patients with pancreatic cancer

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. Nothing herein is to be construed as an admission that the invention is not entitled to antedate such disclosure by virtue of prior invention.

As used herein, the singular forms “a,” “an,” and “the” include plural reference unless the context clearly dictates otherwise. Thus, for example, reference to a “therapeutic” is a reference to one or more therapeutics and equivalents thereof known to those skilled in the art, and so forth.

As used herein, the term “about” means plus or minus 10% of the numerical value of the number with which it is being used. Therefore, about 50% means in the range of 45% to 55%.

“Administering,” when used in conjunction with a therapeutic, means to administer a therapeutic directly into or onto a target tissue or to administer a therapeutic to a patient whereby the therapeutic treats the tissue to which it is targeted. Thus, as used herein, the term “administering,” when used in conjunction with a therapeutic, can include, but is not limited to, providing the therapeutic into or onto the target tissue; providing the therapeutic systemically to a patient by, e.g., intravenous injection whereby the therapeutic reaches the target tissue; providing the therapeutic in the form of the encoding sequence thereof to the target tissue (e.g., by so-called gene-therapy techniques). “Administering” a composition may be accomplished by oral administration, intravenous injection, intraperitoneal injection, intramuscular injection, subcutaneous injection, transdermal diffusion or electrophoresis, local injection, extended release delivery devices including locally implanted extended release devices such as bioerodible or reservoir-based implants, as protein therapeutics or as nucleic acid therapeutic via gene therapy vectors, topical administration, or by any of these methods in combination with other known techniques. Such combination techniques include, without limitation, heating, radiation and ultrasound.

“Agent” as used herein refers to a molecule that specifically binds to a cancer associated sequence or a molecule encoded for by a cancer associated sequence or a receptor that binds to a molecule encoded for by a cancer associated sequence. Examples of agents include nucleic acid molecules, such as DNA and proteins, such as antibodies. The agent may be linked with a label or detectible substance as described infra. The agent may be linked with a therapeutic agent or a toxin.

The term “amplify” as used herein means creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases, or any combination thereof.

The term “animal,” “patient” or “subject” as used herein includes, but is not limited to, humans, non-human primates and non-human vertebrates such as wild, domestic and farm animals including any mammal, such as cats, dogs, cows, sheep, pigs, horses, rabbits, rodents such as mice and rats. In some embodiments, the term “subject,” “patient” or “animal” refers to a male. In some embodiments, the term “subject,” “patient” or “animal” refers to a female.

The term “antibody”, as used herein, means an immunoglobulin or a part thereof, and encompasses any polypeptide comprising an antigen-binding site regardless of the source, method of production, or other characteristics. The term includes for example, polyclonal, monoclonal, monospecific, polyspecific, humanized, single-chain, chimeric, synthetic, recombinant, hybrid, mutated, and CDR-grafted antibodies. A part of an antibody can include any fragment which can bind antigen, for example, an Fab, F (ab′)₂, Fv, scFv.

The term “biological sources” as used herein refers to the sources from which the target polynucleotides or proteins or peptide fragments may be derived. The source can be of any form of “sample” as described infra, including but not limited to, cell, tissue or fluid. “Different biological sources” can refer to different cells/tissues/organs of the same individual, or cells/tissues/organs from different individuals of the same species, or cells/tissues/organs from different species.

The term “capture reagent” refers to a reagent, for example an antibody or antigen binding protein, capable of binding a target molecule or analyte to be detected in a sample.

The term “gene expression result” refers to a qualitative and/or quantitative result regarding the expression of a gene or gene product. Any method known in the art may be used to quantitate a gene expression result. The gene expression result can be an amount or copy number of the gene, the RNA encoded by the gene, the mRNA encoded by the gene, the protein product encoded by the gene, or any combination thereof. The gene expression result can also be normalized or compared to a standard. The gene expression result can be used, for example, to determine if a gene is expressed, overexpressed, or differentially expressed in two or more samples by comparing the gene expression results from 2 or more samples or one or more samples with a standard or a control.

The term “homology,” as used herein, refers to a degree of complementarity. There may be partial homology or complete homology. The word “identity” may substitute for the word “homology.” A partially complementary nucleic acid sequence that at least partially inhibits an identical sequence from hybridizing to a target nucleic acid is referred to as “substantially homologous.” The inhibition of hybridization of the completely complementary nucleic acid sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

As used herein, the term “hybridization” or “hybridizing” refers to hydrogen bonding, which may be Watson-Crick, Hoogsteen or reversed Hoogsteen hydrogen bonding between complementary nucleoside or nucleotide bases. For example, adenine and thymine are complementary nucleobases which pair through the formation of hydrogen bonds. “Complementary,” as used herein in reference to nucleic acid molecules refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that a nucleic acid sequence need not be 100% complementary to that of its target nucleic acid to be specifically hybridizable. A nucleic acid compound is specifically hybridizable when there is binding of the molecule to the target, and there is a sufficient degree of complementarity to avoid non-specific binding of the molecule to non-target sequences under conditions in which specific binding is desired, i.e., under physiological conditions in the case of in vivo assays or therapeutic treatment, and in the case of in vitro assays, under conditions in which the assays are performed.

The term “inhibiting” includes the administration of a compound of the present disclosure to prevent the onset of the symptoms, alleviating the symptoms, or eliminating the disease, condition or disorder. The term “inhibiting” may also refer to lowering the expression level of gene, such as a gene encoding a cancer associated sequence. Expression level of RNA and/or protein may be lowered.

The term “label” and/or detectible substance refer to a composition capable of producing a detectable signal indicative of the presence of the target polynucleotide or a polypeptide or protein in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by a device or method, such as, but not limited to, a spectroscopic, photochemical, biochemical, immunochemical, electrical, optical, chemical detection device or any other appropriate device. In some embodiments, the label may be detectable visually without the aid of a device. The term “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

A “microarray” is a linear or two-dimensional array of, for example, discrete regions, each having a defined area, formed on the surface of a solid support. The density of the discrete regions on a microarray is determined by the total numbers of target polynucleotides to be detected on the surface of a single solid phase support, preferably at least about 50/cm² more preferably at least about 100/cm², even more preferably at least about 500/cm², and still more preferably at least about 1,000/cm². As used herein, a DNA microarray is an array of oligonucleotide primers placed on a chip or other surfaces used to identify, amplify, detect, or clone target polynucleotides. Since the position of each particular group of primers in the array is known, the identities of the target polynucleotides can be determined based on their binding to a particular position in the microarray.

As used herein, the term “naturally occurring” refers to sequences or structures that may be in a form normally found in nature. “Naturally occurring” may include sequences in a form normally found in any animal.

The use of “nucleic acid,” “polynucleotide” or “oligonucleotide” or equivalents herein means at least two nucleotides covalently linked together. In some embodiments, an oligonucleotide is an oligomer of 6, 8, 10, 12, 20, 30 or up to 100 nucleotides. In some embodiments, an oligonucleotide is an oligomer of at least 6, 8, 10, 12, 20, 30, 40, 50, 60, 70, 80, 90, 100, 150, 200, 300, 400, or 500 nucleotides. A “polynucleotide” or “oligonucleotide” may comprise DNA, RNA, PNA or a polymer of nucleotides linked by phosphodiester and/or any alternate bonds.

As used herein, the term “optional” or “optionally” refers to embodiments where the subsequently described structure, event or circumstance may or may not occur, and that the description includes instances where the event occurs and instances where it does not.

The phrases “percent homology,” “% homology,” “percent identity,” or “% identity” refer to the percentage of sequence similarity found in a comparison of two or more amino acid or nucleic acid sequences. Percent identity can be determined electronically, e.g., by using the MEGALIGN program (LASERGENE software package, DNASTAR). The MEGALIGN program can create alignments between two or more sequences according to different methods, e.g., the Clustal Method. (Higgins, D. G. and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be calculated by the Clustal Method, or by other methods known in the art, such as the Jotun Hein Method. (See, e.g., Hein, J. (1990) Methods Enzymol. 183:626-645.) Identity between sequences can also be determined by other methods known in the art, e.g., by varying hybridization conditions.

By “pharmaceutically acceptable”, it is meant the carrier, diluent or excipient must be compatible with the other ingredients of the formulation and not deleterious to the recipient thereof.

“Recombinant protein,” as used herein, means a protein made using recombinant techniques, for example, but not limited to, through the expression of a recombinant nucleic acid as depicted infra. A recombinant protein may be distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises about 50-75%, about 80%, or about 90%. In some embodiments, a substantially pure protein comprises about 80-99%, 85-99%, 90-99%, 95-99%, or 97-99% by weight of the total protein. A recombinant protein can also include the production of a cancer associated protein from one organism (e.g. human) in a different organism (e.g. yeast, E. coli, or the like) or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed herein.

As used herein, the term “sample” refers to composition that is being tested or treated with a reagent, agent, capture reagent, binding partner and the like. Samples may be obtained from subjects. In some embodiments, the sample may be blood, plasma, serum, or any combination thereof. A sample may be derived from blood, plasma, serum, or any combination thereof. Other typical samples include, but are not limited to, any bodily fluid obtained from a mammalian subject, tissue biopsy, sputum, lymphatic fluid, blood cells (e.g., peripheral blood mononuclear cells), tissue or fine needle biopsy samples, urine, peritoneal fluid, colostrums, breast milk, fetal fluid, fecal material, tears, pleural fluid, or cells therefrom. The sample may be processed in some manner before being used in a method described herein, for example a particular component to be analyzed or tested according to any of the methods described infra. One or more molecules may be isolated from a sample.

The terms “specific binding,” “specifically binds,” and the like, refer to instances where two or more molecules form a complex that is measurable under physiologic or assay conditions and is selective. An antibody or antigen binding protein or other molecule is said to “specifically bind” to a protein, antigen, or epitope if, under appropriately selected conditions, such binding is not substantially inhibited, while at the same time non-specific binding is inhibited. Specific binding is characterized by a high affinity and is selective for the compound, protein, epitope, or antigen. Nonspecific binding usually has a low affinity. Examples of specific binding include the binding of enzyme and substrate, an antibody and its antigenic epitope, a cellular signaling molecule and its respective cell receptor.

As used herein, a polynucleotide “derived from” a designated sequence refers to a polynucleotide sequence which is comprised of a sequence of approximately at least about 6 nucleotides, preferably at least about 8 nucleotides, more preferably at least about 10-12 nucleotides, and even more preferably at least about 15-20 nucleotides corresponding to a region of the designated nucleotide sequence. “Corresponding” means homologous to or complementary to the designated sequence. Preferably, the sequence of the region from which the polynucleotide is derived is homologous to or complementary to a sequence that is unique to a cancer associated gene.

As used herein, the term “tag,” “sequence tag” or “primer tag sequence” refers to an oligonucleotide with specific nucleic acid sequence that serves to identify a batch of polynucleotides bearing such tags therein. Polynucleotides from the same biological source are covalently tagged with a specific sequence tag so that in subsequent analysis the polynucleotide can be identified according to its source of origin. The sequence tags also serve as primers for nucleic acid amplification reactions.

The term “support” refers to conventional supports such as beads, particles, dipsticks, fibers, filters, membranes, and silane or silicate supports such as glass slides.

As used herein, the term “therapeutic” or “therapeutic agent” means an agent that can be used to treat, combat, ameliorate, prevent or improve an unwanted condition or disease of a patient. In part, embodiments of the present disclosure are directed to the treatment of cancer or the decrease in proliferation of cells. In some embodiments, the term “therapeutic” or “therapeutic agent” may refer to any molecule that associates with or affects the target marker or cancer associated sequence disclosed infra, its expression or its function. In various embodiments, such therapeutics may include molecules such as, for example, a therapeutic cell, a therapeutic peptide, a therapeutic gene, a therapeutic compound, or the like, that associates with or affects the target marker or cancer associated sequence disclosed infra, its expression or its function.

A “therapeutically effective amount” or “effective amount” of a composition is a predetermined amount calculated to achieve the desired effect, i.e., to inhibit, block, or reverse the activation, migration, metastasis, or proliferation of cells. In some embodiments, the effective amount is a prophylactic amount. In some embodiments, the effective amount is an amount used to medically treat the disease or condition. The specific dose of a composition administered according to this invention to obtain therapeutic and/or prophylactic effects will, of course, be determined by the particular circumstances surrounding the case, including, for example, the composition administered, the route of administration, and the condition being treated. It will be understood that the effective amount administered will be determined by the physician in the light of the relevant circumstances including the condition to be treated, the choice of composition to be administered, and the chosen route of administration. A therapeutically effective amount of composition of this invention is typically an amount such that when it is administered in a physiologically tolerable excipient composition, it is sufficient to achieve an effective systemic concentration or local concentration in the targeted tissue.

The terms “treat,” “treated,” or “treating” as used herein can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “tissue” refers to any aggregation of similarly specialized cells that are united in the performance of a particular function.

Cancer Associated Sequences

In some embodiments, the present disclosure provides for nucleic acid and protein sequences that are associated with cancer, herein termed “cancer associated” or “CA” sequences. In some embodiments, the present disclosure provides nucleic acid and protein sequences that are associated with pancreatic cancers or carcinomas such as, without limitation, carcinoma, any malignant pancreatic neoplasm, ductal adenocarcinoma, cholangiocarcinoma, muinous carcinoma, adenosquamous carcinoma, signet ring cell carcinoma, hepatoid carcinomas, colloid carcinomas, undifferentiated carcinomas, pancreatic cystic neoplasms, islet cell tumors, pancreatic endocrine tumors, pancreatic neuroendocrine carcinoma, extrapulmonary small cell cancer, undifferentiated carcinoma or a combination thereof. The method of diagnosing may comprise measuring the level of expression of a cancer associated marker disclosed herein. The method may further comprise comparing the expression level of the cancer associated sequence with a standard and/or a control. The standard may be from a sample known to contain pancreatic cancer cells. The control may include known pancreatic cancer cells and/or non-cancerous cells, such as non-cancer cells derived from pancreatic tissue.

Cancer associated sequences may include those that are up-regulated (i.e. expressed at a higher level), as well as those that are down-regulated (i.e. expressed at a lower level), in cancers. Cancer associated sequences can also include sequences that have been altered (i.e., translocations, truncated sequences or sequences with substitutions, deletions or insertions, including, but not limited to, point mutations) and show either the same expression profile or an altered profile. In some embodiments, the cancer associated sequences are from humans; however, as will be appreciated by those in the art, cancer associated sequences from other organisms may be useful in animal models of disease and drug evaluation; thus, other cancer associated sequences may be useful, including those obtained from any subject, such as, without limitation, sequences from vertebrates, including mammals, such as rodents (rats, mice, hamsters, guinea pigs, etc.), primates, and farm animals (including sheep, goats, pigs, cows, horses, etc.). Cancer associated sequences from other organisms may be obtained using the techniques outlined herein.

Examples of cancer associated sequences include the genes listed in tables 1, 2, 6 or fragments thereof.

In some embodiments, the cancer associated sequences are nucleic acids. As will be appreciated by those skilled in the art and as described herein, cancer associated sequences of embodiments herein may be useful in a variety of applications including diagnostic applications to detect nucleic acids or their expression levels in a subject, therapeutic applications or a combination thereof. Further, the cancer associated sequences of embodiments herein may be used in screening applications; for example, generation of biochips comprising nucleic acid probes to the cancer associated sequences.

A nucleic acid of the present disclosure may include phosphodiester bonds, although in some cases, as outlined below (for example, in antisense applications or when a nucleic acid is a candidate drug agent), nucleic acid analogs may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996),). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp. 169-176). Several nucleic acid analogs are described in Rawls, C & E News Jun., 2, 1997 page 35. These modifications of the ribose-phosphate backbone may be done for a variety of reasons, for example to increase the stability and half-life of such molecules in physiological environments for use in anti-sense applications or as probes on a biochip.

As will be appreciated by those skilled in the art, such nucleic acid analogs may be used in some embodiments of the present disclosure. In addition, mixtures of naturally occurring nucleic acids and analogs can be made; alternatively, mixtures of different nucleic acid analogs, and mixtures of naturally occurring nucleic acids and analogs may be made.

In some embodiments, the nucleic acids may be single stranded or double stranded or may contain portions of both double stranded or single stranded sequence. As will be appreciated by those skilled in the art, the depiction of a single strand also defines the sequence of the other strand; thus the sequences described herein also includes the complement of the sequence. The nucleic acid may be DNA, both genomic and cDNA, RNA, or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus, for example, the subject units of a peptide nucleic acid, each containing a base, are referred to herein as a nucleoside.

In some embodiments, cancer associated sequences may include both nucleic acid and amino acid sequences. In some embodiments, the cancer associated sequences may include sequences having at least about 60% homology with the disclosed sequences. In some embodiments, the cancer associated sequences may have at least about 65%, about 70%, about 75%, about 80%, about 85%, about 90%, about 95%, about 97%, about 99%, about 99.8% homology with the disclosed sequences. In some embodiments, the cancer associated sequences may be “mutant nucleic acids”. As used herein, “mutant nucleic acids” refers to deletion mutants, insertions, point mutations, substitutions, translocations.

In some embodiments, the cancer associated sequences may be recombinant nucleic acids. By the term “recombinant nucleic acid” herein refers to nucleic acid molecules, originally formed in vitro, in general, by the manipulation of nucleic acid by polymerases and endonucleases, in a form not normally found in nature. Thus a recombinant nucleic acid may also be an isolated nucleic acid, in a linear form, or cloned in a vector formed in vitro by ligating DNA molecules that are not normally joined, are both considered recombinant for the purposes of this invention. It is understood that once a recombinant nucleic acid is made and reintroduced into a host cell or organism, it can replicate using the in vivo cellular machinery of the host cell rather than in vitro manipulations; however, such nucleic acids, once produced recombinantly, although subsequently replicated in vivo, are still considered recombinant or isolated for the purposes of the invention. As used herein, a “polynucleotide” or “nucleic acid” is a polymeric form of nucleotides of any length, either ribonucleotides or deoxyribonucleotides. This term includes double- and single-stranded DNA and RNA. It also includes known types of modifications, for example, labels which are known in the art, methylation, “caps”, substitution of one or more of the naturally occurring nucleotides with an analog, internueleotide modifications-such as, for example, those with uncharged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

The use of microarray analysis of gene expression allows the identification of host sequences associated with pancreatic cancer. These sequences may then be used in a number of different ways, including diagnosis, prognosis, screening for modulators (including both agonists and antagonists), antibody generation (for immunotherapy and imaging), etc. However, as will be appreciated by those skilled in the art, sequences that are identified in one type of cancer may have a strong likelihood of being involved in other types of cancers as well. Thus, while the sequences outlined herein are initially identified as correlated with pancreatic cancers, they may also be found in other types of cancers as well.

Some embodiments described herein may be directed to the use of cancer associated sequences for diagnosis and treatment of pancreatic cancer. In some embodiments, the cancer associated sequence may be selected from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a combination thereof. In some embodiments, these cancer associated sequences may be associated with pancreatic cancers including, without limitation, carcinoma, any malignant pancreatic neoplasm, ductal adenocarcinoma, cholangiocarcinoma, muinous carcinoma, adenosquamous carcinoma, signet ring cell carcinoma, hepatoid carcinomas, colloid carcinomas, undifferentiated carcinomas, pancreatic cystic neoplasms, islet cell tumors, pancreatic endocrine tumors, pancreatic neuroendocrine carcinoma, extrapulmonary small cell cancer, undifferentiated carcinoma or a combination thereof.

In some embodiments, the cancer associated sequences may be DNA sequences encoding the above mRNA or the cancer associated protein or cancer associated polypeptide expressed by the above mRNA or homologs thereof. In some embodiments, the cancer associated sequence may be a mutant nucleic acid of the above disclosed sequences. In some embodiments, the homolog may have at least about 60%, at least about 65%, at least about 70%, at least about 75%, at least about 80%, at least about 85%, at least about 90%, at least about 95%, at least about 97%, at least about 98%, at least about 99%, at least about 99.5% identity with the disclosed polypeptide sequence.

In some embodiments, an isolated nucleic acid comprises at least 10, 12, 15, 20 or 30 contiguous nucleotides of a sequence selected from the group consisting of the cancer associated polynucleotide sequences disclosed in tables 1, 2 and 6.

In some embodiments, the polynucleotide, or its complement or a fragment thereof, further comprises a detectable label, is attached to a solid support, is prepared at least in part by chemical synthesis, is an antisense fragment, is single stranded, is double stranded or comprises a microarray.

In some embodiments, the invention provides an isolated polypeptide, encoded within an open reading frame of a cancer associated sequence selected from the polynucleotide sequences encoded by the genes disclosed in Tables 1, 2, and 6, or its complement. In some embodiments, the invention provides an isolated polypeptide, wherein said polypeptide comprises the amino acid sequence encoded by a polynucleotide selected from the group consisting of sequences encoded by the genes disclosed in Tables 1, 2, and 6. In some embodiments, the invention provides an isolated polypeptide, wherein said polypeptide comprises the amino acid sequence encoded by a cancer associated polypeptide as described infra.

In some embodiments, the invention further provides an isolated polypeptide, comprising the amino acid sequence of an epitope of the amino acid sequence of a cancer associated polypeptide disclosed infra. The polypeptide or fragment thereof may be attached to a solid support. In some embodiments the invention provides an isolated antibody (monoclonal or polyclonal) or antigen binding fragment thereof, that binds to such a polypeptide. The isolated antibody or antigen binding fragment thereof may be attached to a solid support. The isolated antibody or antigen binding fragment thereof may further comprise a detectable substance.

Some embodiments also provide for antigens (e.g., cancer-associated polypeptides) associated with a variety of cancers as targets for diagnostic and/or therapeutic antibodies, e.g. pancreatic cancer. These antigens may also be useful for drug discovery (e.g., small molecules) and for further characterization of cellular regulation, growth, and differentiation.

### Methods of Detecting and Diagnosing Pancreatic Cancer

In some embodiments, the method of detecting or diagnosing pancreatic cancer may comprise assaying gene expression of a subject in need thereof. Any method known in the art may be used to assay gene expression of one or more markers disclosed infra. In some embodiments, detecting a level of a cancer associated sequence may comprise techniques such as, but not limited to, PCR, mass spectroscopy, microarray, gel electrophoresis, hybridization using one more probes that specifically bind a nucleic acid encoding a cancer associated sequence disclosed infra. Information relating to expression of the receptor can also be useful in determining therapies aimed at up or down-regulating the cancer associated sequence's signaling using agonists or antagonists.

In some embodiments the invention provides a method of diagnosing pancreatic cancer in a subject comprising a) obtaining a sample, such as a bodily fluid from a subject b) contacting the sample with an agent which detects a gene product encoded for by one or more genes chosen from UBD, LAMC2, PPY, REG4, REG1b, LCN2, MMP11, COL10A and MMP7, c) contacting a sample that is known to be free of pancreatic cancer with the agent from b) and d) comparing the level of the gene product encoded for by one or more genes chosen from UBD, LAMC2, PPY, REG4, REG1b, LCN2, MMP11, COL10A and MMP7 in the sample with the level of expression of gene product encoded for by one or more genes chosen from UBD, LAMC2, PPY, REG4, REG1b, LCN2, MMP11, COL10A and MMP7 in the sample that is known to be free of pancreatic cancer, wherein an elevated level of the one or more gene products encoded for by one or more genes chosen from UBD, LAMC2, PPY, REG4, REG1b, LCN2, MMP11, COL10A and MMP7 in the sample obtained from the subject compared to the sample known to be free of pancreatic cancer indicates that the subject has pancreatic cancer. The gene product may be for example a protein or peptide or an mRNA encoded for by one or more genes chosen from UBD, LAMC2, PPY, REG4, REG1b, LCN2, MMP11, COL10A and MMP7. The bodily fluid may be blood, serum, plasma or the like.

In some embodiments the invention provides a method of diagnosing pancreatic cancer in a subject comprising a) obtaining a sample, such as a bodily fluid from a subject b) contacting the sample with one or more agents which detects a panel of markers encoded for by the genes UBD, LAMC2, PPY, REG4, REG1b, LCN2, MMP11, COL10A and MMP7, c) contacting a sample that is known to be free of pancreatic cancer with the one or more agents from b) and d) comparing the level of the gene products encoded by the genes UBD, LAMC2, PPY, REG4, REG1b, LCN2, MMP11, COL10A and MMP7 in the sample with the level of expression of gene product encoded for by the genes UBD, LAMC2, PPY, REG4, REG1b, LCN2, MMP11, COL10A and MMP7 in the sample that is known to be free of pancreatic cancer, wherein an elevated level of at least one of gene products encoded for by the genes UBD, LAMC2, PPY, REG4, REG1b, LCN2, MMP11, COL10A and MMP7 in the sample obtained from the subject compared to the sample known to be free of pancreatic cancer indicates that the subject has pancreatic cancer. The gene product may be for example a protein or peptide or an mRNA (or a combination thereof) encoded for by the genes chosen from UBD, LAMC2, PPY, REG4, REG1b, LCN2, MMP11, COL10A and MMP7. The bodily fluid may be blood, serum, plasma or the like.

In some embodiments, a method of diagnosing pancreatic cancer may comprise detecting a level of the cancer associated protein in a subject. In some embodiments, a method of screening for cancer may comprise detecting a level of the cancer associated protein. In some embodiments, the cancer associated protein is encoded by a nucleotide sequence selected from one or more genes disclosed in Tables 1, 2 and 6, a fragment thereof or a complementary sequence thereof. In some embodiments, a method of detecting cancer in a sample may comprise contacting the sample obtained from a subject with an antibody that specifically binds the protein. In some embodiments, the antibody may be a monoclonal antibody or a polyclonal antibody. In some embodiments, the antibody may be a humanized or a recombinant antibody. Antibodies can be made that specifically bind to this region using known methods and any method is suitable. In some embodiments, the antibody specifically binds to one or more of a molecule, such as protein or peptide, encoded for by one or more cancer associated sequences disclosed infra.

In some embodiments, the antibody binds to an epitope from a protein encoded by the nucleotide sequence of the genes listed in tables 1, 2, 6 or fragments thereofwith an antibody against the protein. In some embodiments, the epitope is a fragment of the protein sequence encoded by the nucleotide sequence of any of the cancer associated sequences disclosed infra. In some embodiments, the epitope comprises about 1-10, 1-20, 1-30, 3-10, or 3-15 residues of the cancer associated sequence. In some embodiments, the epitope is not linear.

In some embodiments, the antibody binds to the regions described herein or a peptide with at least 90, 95, or 99% homology or identity to the region. In some embodiments, the fragment of the regions described herein is 5-10 residues in length. In some embodiments, the fragment of the regions (e.g. epitope) described herein are 3-5 residues in length. The fragments are described based upon the length provided. In some embodiments, the epitope is about 3, 4, 5, 6, 7, 8, 9, 10, 11, 12, 13, 14, 15, or 20 residues in length.

In some embodiments, the sequence to which the antibody binds may include both nucleic acid and amino acid sequences. In some embodiments, the sequence to which the antibody binds may include sequences having at least about 60% homology with the disclosed sequences. In some embodiments, the sequence to which the antibody binds may have at least about 65%, about 70%, about 75%, about 80%, about 85%, about 90%, about 95%, about 97%, about 99%, about 99.8% homology with the disclosed sequences. In some embodiments, the sequences may be referred to as “mutant nucleic acids” or “mutant peptide sequences.”

In some embodiments, a subject can be diagnosed with pancreatic cancer by detecting the presence of a cancer associated sequence (e.g. the genes listed in tables 1, 2, 6 or fragments thereof) in a sample obtained from a subject. In some embodiments, the method comprises detecting the presence or absence of a cancer associated sequence selected from sequences encoding the genes disclosed in tables 1, 2, or 6, wherein the absence of the cancer associated sequence indicates that absence of pancreatic cancer. In some embodiments, the method further comprises treating the subject diagnosed with pancreatic cancer with an antibody that binds to a cancer associated sequence disclosed infra and inhibits the growth or progression of the pancreatic cancer. As discussed, pancreatic cancer may be detected in any type of sample, including, but not limited to, serum, blood, tumor and the like. The sample may be any type of sample as it is described herein.

Any assay suitable may be used to screen for the presence, absence or expression level of one or more proteins encoded for by a cancer associated sequence described infra. In some embodiments the assay may be for example an ELISA, a radio-immuno assay, a western blot, a flow cytometry assay and the like.

In some embodiments, the method of diagnosing a subject with pancreatic cancer comprises obtaining a sample and detecting the presence of a cancer associated sequence selected from sequences encoding the genes listed in tables 1, 2, 6 or fragments thereof, wherein the presence of the cancer associated sequence indicates the subject has pancreatic cancer. In some embodiments, detecting the presence of a cancer associated sequence selected from sequences disclosed infra comprises contacting the sample with an antibody or other type of capture reagent or specific binding partner that specifically binds to the cancer associated sequence's protein and detecting the presence or absence of the binding to the cancer associated sequence's protein in the sample.

In some embodiments, the present disclosure provides a method of diagnosing pancreatic cancer, or a neoplastic condition in a subject, the method comprising obtaining a cancer associated sequence gene expression result of a cancer associated sequence selected from sequences disclosed infra from a sample derived from a subject; and diagnosing pancreatic cancer or a neoplastic condition in the subject based on the cancer associated sequence gene expression result, wherein the subject is diagnosed as having pancreatic cancer or a neoplastic condition if the cancer associated sequence is expressed at a level that is 1) higher than a negative control such a non-cancerous pancreatic tissue or cell sample and/or 2) higher than or equivalent to the expression level of the cancer associated sequence in a standard or positive control wherein the standard or positive control is known to contain pancreatic cancer cells.

Some embodiments are directed to a biochip comprising one or more nucleic acid sequences which encodeone or more cancer associated proteins. In some embodiments, a biochip comprises a nucleic acid molecule which encodes at least a portion of a cancer associated protein. In some embodiments, the cancer associated protein is encoded by a sequence selected from one or more genes disclosed in tables 1, 2, and 6, homologs thereof, combinations thereof, or a fragment thereof. In some embodiments, the nucleic acid molecule specifically hybridizes with a nucleic acid sequence encoded for by one or more genes disclosed in tables 1, 2, and 6. In some embodiments, the biochip comprises a first and second nucleic molecule wherein the first nucleic acid molecule specifically hybridizes with a first sequence selected from cancer associated sequences disclosed infra and the second nucleic acid molecule specifically hybridizes with a second sequence selected from cancer associated sequences disclosed infra, wherein the first and second sequences are not the same sequence. In some embodiments, the present invention provides methods of detecting or diagnosing cancer, such as pancreatic cancer, comprising detecting the expression of a nucleic acid sequence selected from a sequence encoding the genes listed in tables 1, 2, 6 or fragments thereof, wherein a sample is contacted with a biochip comprising a sequence selected from sequences encoding the genes listed in tables 1, 2, 6 or fragments thereof, homologs thereof, combinations thereof, or a fragment thereof.

Also provided herein is a method for diagnosing or determining the propensity to cancers, for example pancreatic cancer, by measuring the expression level of one or more of the cancer associated sequences disclosed infra in a sample and comparing the expression level of the one or more cancer associated sequences in the sample with expression level of the same cancer associated sequences in a non-cancerous cell. A higher level of expression of one or more of the cancer associated sequences disclosed infra compared to the non-cancerous cell indicates a propensity for the development of cancer, e.g., pancreatic cancer.

In some embodiments, the invention provides a method for detecting a cancer associated sequence with the expression of a polypeptide in a test sample, comprising detecting a level of expression of at least one polypeptide such as, without limitation, a cancer associated protein encoded for by a sequence disclosed infra, or a fragment thereof. In some embodiments, the method comprises comparing the level of expression of the polypeptide in the test sample with a level of expression of polypeptide in a normal sample, i.e. a non-cancerous sample, wherein an altered level of expression of the polypeptide in the test sample relative to the level of polypeptide expression in the normal sample is indicative of the presence of cancer in the test sample. In some embodiments, the polypeptide expression is compared to a cancer sample, wherein the level of expression is at least the same as the cancer is indicative of the presence of cancer in the test sample. In some embodiments the test sample is compared to a normal, e.g. a non-cancerous sample where an expression level in the test sample that is greater than that found in the normal sample indicates the presence of cancer in the test sample. In some embodiments, the sample is a cell sample. In some embodiments the sample is a tissue sample. In some embodiments the sample is a bodily fluid. Examples of suitable bodily fluids, include, but are not limited to, blood, serum, saliva or urine. In some embodiments the sample is a blood sample. In some embodiments the sample is a serum sample. In some embodiments the sample is a urine sample.

In some embodiments, the invention provides a method for detecting cancer by detecting the presence of an antibody in a test serum sample. In some embodiments, the antibody recognizes a polypeptide or an epitope of a cancer associated sequence disclosed herein. In some embodiments, the method comprises detecting a level of an antibody against an antigenic polypeptide such as, without limitation, a cancer associated protein such as a protein encoded for by a cancer associated sequence disclosed infra, or an antigenic fragment thereof. In some embodiments, the method comprises comparing the level of the antibody in the test sample with a level of the antibody in the control sample, wherein an altered level of antibody in said test sample relative to the level of antibody in the control sample is indicative of the presence of cancer in the test sample. In some embodiments, the control sample is a sample derived from a non-cancerous sample e.g. blood or serum obtained from a subject that is cancer free. In some embodiments, the control is derived from a cancer sample, and, therefore, in some embodiments, the method comprises comparing the levels of binding and/or the amount of antibody in the sample, wherein when the levels or amount are the same as the cancer control sample is indicative of the presence of cancer in the test sample.

In some embodiments, a method for diagnosing cancer or a neoplastic condition comprises a) determining the expression of one or more genes comprising a nucleic acid sequence selected from the group consisting of the human genomic and mRNA sequences encoding the genes listed in tables 1, 2, 6 or fragments thereof, in a first sample type (e.g. tissue, bodily fluid, etc.) of a first individual; and b) comparing said expression of said gene(s) from a second normal sample type from said first individual or a second unaffected individual; wherein a difference in said expression indicates that the first individual has cancer. In some embodiments, the expression is increased as compared to the normal sample.

In some embodiments, the invention also provides a method for detecting presence or absence of cancer cells in a subject. In some embodiments, the method comprises contacting one or more cells from the subject with an antibody as described herein. The antibody may be conjugated to a detectible substance. In some embodiments the antibody that binds to a protein encoded for by a cancer associated sequence disclosed infra may bind to a second antibody wherein the second antibody is conjugated to a detectible substance. In some embodiments the antibody that binds to a protein encoded for by a cancer associated sequence disclosed infra is bound to a solid support. In some embodiments, the method comprises detecting a complex of a cancer associated protein and the antibody, wherein detection of the complex indicates with the presence of cancer cells in the subject. The complex may include a detectable substance as described infra. The complex may include a solid support, such as bead, a chip, a magnet, a multiwell plate and the like.

In some embodiments, the present disclosure provides methods of detecting cancer in a test sample, comprising: (i) detecting a level of activity of at least one polypeptide that is a gene product; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample, wherein said gene product is a product of a gene selected from one or more of the cancer associated sequences provided infra.

Capture Reagents and Specific Binding Partners

The invention provides for specific binding partners and capture reagents that bind specifically to cancer associated sequences disclosed infra and the polypeptides or proteins encoded for by those sequences. The capture reagents and specific binding partners may be used in diagnostic assays as disclosed infra and/or in therapeutic methods described infra as well as in drug screening assays disclosed infra. Capture reagents include for example nucleic acids and proteins. Suitable proteins include antibodies.

As used herein, the term “specifically binds” or “specifically binding” means binding that is measurably different from a non-specific interaction. Specific binding can be measured, for example, by determining binding of a molecule compared to binding of a control molecule, which generally is a molecule of similar structure that does not have binding activity. For example, specific binding is indicated if the molecule has measurably higher affinity for cells expressing a protein encoded for by a cancer associated sequence disclosed infra than for cells that do not express the same protein encoded for by the cancer associated sequences disclosed infra. Specificity of binding can be determined, for example, by competitive inhibition of a known binding molecule.

The term “specifically binding,” as used herein, includes both low and high affinity specific binding. Specific binding can be exhibited, for example, by a low affinity homing molecule having a Kd of at least about 10⁻⁴ M. Specific binding also can be exhibited by a high affinity homing molecule, for example, a homing molecule having a Kd of at least about 10⁻⁵M. Such a molecule can have, for example, a Kd of at least about 10^{-6 M}, at least about 10⁻⁷ M, at least about 10⁻⁸ M, at least about 10⁻⁹ M, at least about 10^{-10 M}, or can have a Kd of at least about 10⁻¹¹ M or 10⁻¹²M or greater. Both low and high affinity homing molecules are useful and are encompassed by the invention. Low affinity homing molecules are useful in targeting, for example, multivalent conjugates. High affinity homing molecules are useful in targeting, for example, multivalent and univalent conjugates.

In some embodiments the specific binding partner or capture reagent is an antibody. Binding in IgG antibodies, for example, is generally characterized by an affinity of at least about 10⁻⁷ M or higher, such as at least about 10⁻⁸ M or higher, or at least about 10⁻⁹ M or higher, or at least about 10⁻¹⁰ or higher, or at least about 10⁻¹¹ M or higher, or at least about 10⁻¹² M or higher. The term is also applicable where, e.g., an antigen-binding domain is specific for a particular epitope that is not carried by numerous antigens, in which case the antibody or antigen binding protein carrying the antigen-binding domain will generally not bind other antigens. In some embodiments, the capture reagent has a Kd equal or less than 10⁻⁹ M, 10⁻¹⁰ M, or 10⁻¹¹ M for its binding partner (e.g. antigen). In some embodiments, the capture reagent has a Ka greater than or equal to 10⁹ M⁻¹ for its binding partner. Capture reagent can also refer to, for example, antibodies. Intact antibodies, also known as immunoglobulins, are typically tetrameric glycosylated proteins composed of two light (L) chains of approximately 25 kDa each, and two heavy (H) chains of approximately 50 kDa each. Two types of light chain, termed lambda and kappa, exist in antibodies. Depending on the amino acid sequence of the constant domain of heavy chains, immunoglobulins are assigned to five major classes: A, D, E, G, and M, and several of these may be further divided into subclasses (isotypes), e.g., IgG1, IgG2, IgG3, IgG4, IgA1, and IgA2. Each light chain is composed of an N-terminal variable (V) domain (VL) and a constant (C) domain (CL). Each heavy chain is composed of an N-terminal V domain (VH), three or four C domains (CHs), and a hinge region. The CH domain most proximal to VH is designated CH1. The VH and VL domains consist of four regions of relatively conserved sequences named framework regions (FR1, FR2, FR3, and FR4), which form a scaffold for three regions of hypervariable sequences (complementarily determining regions, CDRs). The CDRs contain most of the residues responsible for specific interactions of the antibody or antigen binding protein with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is the greatest source of molecular diversity within the antibody or antigen binding protein-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, Eds. Harlow et al., 1988. One of skill in the art will recognize that each subunit structure, e.g., a CH, VH, CL, VL, CDR, and/or FR structure, comprises active fragments. For example, active fragments may consist of the portion of the VL, or CDR subunit that binds the antigen, i.e., the antigen-binding fragment, or the portion of the CH subunit that binds to and/or activates an Fe receptor and/or complement.

Non-limiting examples of binding fragments encompassed within the term “antigen-specific antibody” used herein include: (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VH domain; and (vi) an isolated CDR. Furthermore, although the two domains of the Fv fragment, VL and VH, are coded for by separate genes, they may be recombinantly joined by a synthetic linker, creating a single protein chain in which the VL and VH domains pair to form monovalent molecules (known as single chain Fv (scFv)). The most commonly used linker is a 15-residue (Gly₄Ser)₃ peptide, but other linkers are also known in the art. Single chain antibodies are also intended to be encompassed within the terms “antibody or antigen binding protein,” or “antigen-binding fragment” of an antibody. The antibody can also be a polyclonal antibody, monoclonal antibody, chimeric antibody, antigen-binding fragment, Fc fragment, single chain antibodies, or any derivatives thereof.

Antibodies can be obtained using conventional techniques known to those skilled in the art, and the fragments are screened for utility in the same manner as intact antibodies. Antibody diversity is created by multiple germline genes encoding variable domains and a variety of somatic events. The somatic events include recombination of variable gene segments with diversity (D) and joining (J) gene segments to make a complete VH domain, and the recombination of variable and joining gene segments to make a complete VL domain. The recombination process itself is imprecise, resulting in the loss or addition of amino acids at the V (D) J junctions. These mechanisms of diversity occur in the developing B cell prior to antigen exposure. After antigenic stimulation, the expressed antibody genes in B cells undergo somatic mutation. Based on the estimated number of germ line gene segments, the random recombination of these segments, and random VH-VL pairing, up to 1.6×10⁷ different antibodies may be produced (Fundamental Immunology, 3rd ed. (1993), ed. Paul, Raven Press, New York, N.Y.). When other processes that contribute to antibody diversity (such as somatic imitation) are taken into account, it is thought that upwards of 1×10¹⁰ different antibodies may be generated (Immunoglobulin Genes, 2nd ed. (1995), eds. Jonio et al., Academic Press, San Diego, Calif.). Because of the many processes involved in generating antibody diversity, it is unlikely that independently derived monoclonal antibodies with the same antigen specificity will have identical amino acid sequences.

Antibody or antigen binding protein molecules capable of specifically interacting with the antigens, epitopes, or other molecules described herein may be produced by methods well known to those skilled in the art. For example, monoclonal antibodies can be produced by generation of hybridomas in accordance with known methods. Hybridomas formed in this manner can then be screened using standard methods, such as enzyme-linked immunosorbent assay (ELISA) and Biacore analysis, to identify one or more hybridomas that produce an antibody that specifically interacts with a molecule or compound of interest. As an alternative to preparing monoclonal antibody-secreting hybridomas, a monoclonal antibody to a polypeptide of the present disclosure may be identified and isolated by screening a recombinant combinatorial immunoglobulin library (e.g., an antibody phage display library) with a polypeptide of the present disclosure to thereby isolate immunoglobulin library members that bind to the polypeptide. Techniques and commercially available kits for generating and screening phage display libraries are well known to those skilled in the art. Additionally, examples of methods and reagents particularly amenable for use in generating and screening antibody or antigen binding protein display libraries can be found in the literature.

Examples of chimeric antibodies include, but are not limited to, humanized antibodies. The antibodies described herein can also be human antibodies. In some embodiments, the capture reagent comprises a detection reagent. The detection reagent can be any reagent that can be used to detect the presence of the capture reagent binding to its specific binding partner. The capture reagent can comprise a detection reagent directly or the capture reagent can comprise a particle that comprises the detection reagent. In some embodiments, the capture reagent and/or particle comprises a color, colloidal gold, radioactive tag, fluorescent tag, or a chemiluminescent substrate. The particle can be, for example, a viral particle, a latex particle, a lipid particle, or a fluorescent particle.

The capture reagents (e.g. antibody) of the present disclosure can also include an anti-antibody, i.e. an antibody that recognizes another antibody but is not specific to an antigen, such as, but not limited to, anti-IgG, anti-IgM, or ant-IgE antibody. This non-specific antibody can be used as a positive control to detect whether the antigen specific antibody is present in a sample.

Nucleic acid capture reagents include DNA, RNA and PNA molecules for example. The nucleic acid may be about 5 nucleotides long, about 10 nucleotides long, about 15 nucleotides long, about 20 nucleotides long, about 25 nucleotides long, about 30 nucleotides long, about 35 nucleotides long about 40 nucleotides long. The nucleic acid may be greater than 30 nucleotides long. The nucleic acid may be less than 30 nucleotides long.

Treatment of Pancreatic Cancer

In some embodiments, pancreatic cancers expressing one of the cancer associated sequences disclosed infra may be treated by antagonizing the cancer associated sequence's activity. In some embodiments, a method of treating pancreatic cancer may comprise administering a therapeutic such as, without limitation, antibodies that antagonize the ligand binding to the cancer associated sequence, small molecules that inhibit the cancer associated sequence's expression or activity, siRNAs directed towards the cancer associated sequence, or the like.

In some embodiments, a method of treating cancer (e.g. pancreatic or other types of cancer) comprises detecting the presence of a cancer associated sequence's receptor and administering a cancer treatment. The treatment may specifically bind to the cancer associated sequence's receptor. The cancer treatment may be any cancer treatment or one that is specific to the inhibiting the action of a cancer associated sequence. For example, various cancers are tested to determine if a specific molecule is present before giving a cancer treatment. In some embodiments, therefore, a sample would be obtained from the patient and tested for the presence of a cancer associated sequence or the overexpression of a cancer associated sequence as described herein. In some embodiments, if a cancer associated sequence is found to be overexpressed then a pancreatic cancer treatment or therapeutic is administered to the subject. The pancreatic cancer treatment may be a conventional non-specific treatment, such as chemotherapy, or the treatment may comprise a specific treatment that only targets the activity of the cancer associated sequence or the receptor to which the cancer associated sequence binds. These treatments can be, for example, an antibody that specifically binds to the cancer associated sequence and inhibits its activity. The treatment may be a nucleic acid that downregulates or silences the expression of the cancer associated sequence.

Some embodiments herein describe method of treating cancer or a neoplastic condition comprising administering an antibody against the cancer associated sequence to a subject. In some embodiments, the antibody may be monoclonal or polyclonal. In some embodiments, the antibody may be humanized or recombinant. In some embodiments, the antibody may neutralize biological activity of the cancer associated sequence by binding to and/or interfering with the cancer associated sequence's receptor. In some embodiments the antibody may bind to site on the protein encoded for by the cancer associated DNA sequence that is not the receptor. In some embodiments, administering the antibody may be to a biological fluid or tissue, such as, without limitation, blood, urine, serum, tumor tissue, or the like.

In some embodiments, a method of treating cancer may comprise administering an agent that interferes with the synthesis, secretion, receptor binding or receptor signaling of cancer associated proteins or its receptors. In some embodiments, the cancer may be selected from, including, without limitation, carcinoma, any malignant pancreatic neoplasm, ductal adenocarcinoma, cholangiocarcinoma, muinous carcinoma, adenosquamous carcinoma, signet ring cell carcinoma, hepatoid carcinomas, colloid carcinomas, undifferentiated carcinomas, pancreatic cystic neoplasms, islet cell tumors, pancreatic endocrine tumors, pancreatic neuroendocrine carcinoma, extrapulmonary small cell cancer, undifferentiated carcinoma or a combination thereof.

In some embodiments, the cancer cell may be targeted specifically with a therapeutic based upon the differentially expressed gene or gene product. For example, in some embodiments, the differentially expressed gene product may be an enzyme, which can convert an anti-cancer prodrug into its active form. Therefore, in normal cells, where the differentially expressed gene product is not expressed or expressed at significantly lower levels, the prodrug may be either not activated or activated in a lesser amount, and may be, therefore less toxic to normal cells. Therefore, the cancer prodrug may, in some embodiments, be given in a higher dosage so that the cancer cells can metabolize the prodrug, which will, for example, kill the cancer cell, and the normal cells will not metabolize the prodrug or not as well, and, therefore, be less toxic to the patient. An example of this is where tumor cells overexpress a metalloprotease, which is described in Atkinson et al., British Journal of Pharmacology (2008) 153, 1344-1352. Using proteases to target cancer cells is also described in Carl et al., PNAS, Vol. 77, No. 4, pp. 2224-2228, April 1980. For example, doxorubicin or other type of chemotherapeutic can be linked to a peptide sequence that is specifically cleaved or recognized by the differentially expressed gene product. The doxorubicin or other type of chemotherapeutic is then cleaved from the peptide sequence and is activated such that it can kill or inhibit the growth of the cancer cell whereas in the normal cell the chemotherapeutic is never internalized into the cell or is not metabolized as efficiently, and is, therefore, less toxic.

In some embodiments, a method of treating pancreatic cancer may comprise gene knockdown of one or more cancer associated sequences described herein. Gene knockdown refers to techniques by which the expression of one or more of an organism's genes is reduced, either through genetic modification (a change in the DNA of one of the organism's chromosomes such as, without limitation, chromosomes encoding cancer associated sequences) or by treatment with a reagent such as a short DNA or RNA oligonucleotide with a sequence complementary to either an mRNA transcript or a gene. In some embodiments, the oligonucleotide used may be selected from RNase-H competent antisense, such as, without limitation, ssDNA oligonucleotides, ssRNA oligonucleotides, phosphorothioate oligonucleotides, or chimeric oligonucleotides; RNase-independent antisense, such as morpholino oligonucleotides, 2′-O-methyl phosphorothioate oligonucleotides, locked nucleic acid oligonucleotides, or peptide nucleic acid oligonucleotides; RNAi oligonucleotides, such as, without limitation, siRNA duplex oligonucleotides, or ssRNA oligonucleotides; or any combination thereof. In some embodiments, a plasmid may be introduced into a cell, wherein the plasmid expresses either an antisense RNA transcript or an shRNA transcript. The oligo introduced or transcript expressed may interact with the target mRNA (ex. sequences disclosed in Table 1) by complementary base pairing (a sense-antisense interaction).

The specific mechanism of silencing may vary with the oligo chemistry. In some embodiments, the binding of a oligonucleotide described herein to the active gene or its transcripts may cause decreased expression through blocking of transcription, degradation of the mRNA transcript (e.g. by small interfering RNA (siRNA) or RNase-H dependent antisense) or blocking either mRNA translation, pre-mRNA splicing sites or nuclease cleavage sites used for maturation of other functional RNAs such as miRNA (e.g. by Morpholino oligonucleotides or other RNase-H independent antisense). For example, RNase-H competent antisense oligonucleotides (and antisense RNA transcripts) may form duplexes with RNA that are recognized by the enzyme RNase-H, which cleaves the RNA strand. As another example, RNase-independent oligonucleotides may bind to the mRNA and block the translation process. In some embodiments, the oligonucleotides may bind in the 5′-UTR and halt the initiation complex as it travels from the 5′-cap to the start codon, preventing ribosome assembly. A single strand of RNAi oligonucleotides may be loaded into the RISC complex, which catalytically cleaves complementary sequences and inhibits translation of some mRNAs bearing partially-complementary sequences. The oligonucleotides may be introduced into a cell by any technique including, without limitation, electroporation, microinjection, salt-shock methods such as, for example, CaCl2 shock; transfection of anionic oligo by cationic lipids such as, for example, Lipofectamine; transfection of uncharged oligonucleotides by endosomal release agents such as, for example, Endo-Porter; or any combination thereof. In some embodiments, the oligonucleotides may be delivered from the blood to the cytosol using techniques selected from nanoparticle complexes, virally-mediated transfection, oligonucleotides linked to octaguanidinium dendrimers (Morpholino oligonucleotides), or any combination thereof.

In some embodiments, a method of treating pancreatic cancer may comprise treating a subject with a suitable reagent to knockdown or inhibit expression of a gene encoding the mRNA encoding the genes listed in tables 1, 2, 6 or fragments thereof, or a combination thereof. In other embodiments the invention provides for the in vitro knockdown of the expression of one or more of the genes listed in tables 1, 2, 6 or fragments, for example in an in vitro culture of cells or cells obtained from a sample obtained from a subject.

The method may comprise culturing hES cell-derived clonal embryonic progenitor cell lines CM02 and EN13 (see U.S. Patent Publication 2008/0070303, entitled “Methods to accelerate the isolation of novel cell strains from pluripotent stem cells and cells obtained thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”) with a retrovirus expressing silencing RNA directed to a cancer-associated sequence. In some embodiments, the method may further comprise confirming down-regulation by qPCR. In some embodiments, the method further comprises cryopreserving the cells. In some embodiments, the method further comprises reprogramming the cells. In some embodiments, the method comprises cryopreserving or reprogramming the cells within two days by the exogenous administration of OCT4, MYC, KLF4, and SOX2 (see Takahashi and Yamanaka 2006 Aug. 25; 126(4):663-76; U.S. patent application Ser. No. 12/086,479, published as US2009/0068742 and entitled “Nuclear Reprogramming Factor”) and by the method described in PCT/US06/30632, published as WO/2007/019398 and entitled “Improved Methods of Reprogramming Animal Somatic Cells”. In some embodiments, the method may comprise culturing mammalian differentiated cells under conditions that promote the propagation of ES cells. In some embodiments, any convenient ES cell propagation condition may be used, e.g., on feeders or in feeder free media capable of propagating ES cells. In some embodiments, the method comprises identifying cells from ES colonies in the culture. Cells from the identified ES colony may then be evaluated for ES markers, e.g., Oct4, TRA 1-60, TRA 1-81, SSEA4, etc., and those having ES cell phenotype may be expanded. Control lines that have not been preconditioned by the knockdown may be reprogrammed in parallel to demonstrate the effectiveness of the preconditioning.

In some embodiments, the cancers treated by modulating the activity or expression of sequences of the genes listed in tables 1, 2, 6 or fragments thereof or the gene product thereof.

In some embodiments, a method of treating cancer comprises administering an antibody (e.g. monoclonal antibody, human antibody, humanized antibody, recombinant antibody, chimeric antibody, and the like) that specifically binds to a cancer associated protein that is expressed on a cell surface. In some embodiments, the antibody binds to an extracellular domain of the cancer associated protein. In some embodiments, the antibody binds to a cancer associated protein differentially expressed on a cancer cell surface relative to a normal cell surface, or, in some embodiments, to at least one human cancer cell line. In some embodiments, the antibody is linked to a therapeutic agent or a toxin.

In some embodiments, implementation of an immunotherapy strategy for treating, reducing the symptoms of, or preventing cancer or neoplasms, (e.g., a vaccine) may be achieved using many different techniques available to the skilled artisan.

Immunotherapy or the use of antibodies for therapeutic purposes has been used in recent years to treat cancer. Passive immunotherapy involves the use of monoclonal antibodies in cancer treatments. See, for example, Cancer: Principles and Practice of Oncology, 6 Th Edition (2001) Chapt. 20 pp. 495-508. Inherent therapeutic biological activity of these antibodies include direct inhibition of tumor cell growth or survival, and the ability to recruit the natural cell killing activity of the body's immune system. These agents may be administered alone or in conjunction with radiation or chemotherapeutic agents. Alternatively, antibodies may be used to make antibody conjugates where the antibody is linked to a toxic agent and directs that agent to the tumor by specifically binding to the tumor.

Screening for Cancer Therapeutics

The invention provides for screening assays to determine if a candidate molecule has an inhibitory effect on the growth and or metastasis of pancreatic cancer cells. Suitable candidates include proteins, peptides, nucleic acids such as DNA, RNA shRNA sm RNA and the like, small molecules including small organic molecules and small inorganic molecules. A small molecule may include molecules less than 50 kd.

In some embodiments, a method of identifying an anti-cancer agent is provided, wherein the method comprises contacting a candidate agent to a sample; and determining the cancer associated sequence's activity in the sample, In some embodiments, the candidate agent is identified as an anti-cancer agent if the cancer associated sequence's activity is reduced in the sample after the contacting. In other embodiments the candidate agent reduces the expression level of one or more cancer associated sequences disclosed infra.

In some embodiments, the candidate agent is an antibody. In some embodiments, the method comprises contacting a candidate antibody that binds to the cancer associated sequence with a sample, and assaying for the cancer associated sequence's activity, wherein the candidate antibody is identified as an anti-cancer agent if the cancer associated sequence activity is reduced in the sample after the contacting. A cancer associated sequence's activity can be any activity of the cancer associated sequence. An example of an activity may include inhibiting enzymatic activity either of the cancer associated sequence itself or of an enzyme that interacts with or is modulated by the cancer associated sequence either at the nucleic acid level or the protein level.

In some embodiments, the present disclosure provides methods of identifying an anti-cancer (e.g. pancreatic cancer) agent comprising contacting a candidate agent to a cell sample; and determining activity of a cancer associated sequence, wherein the candidate agent is identified as an anti-cancer agent if the cancer associated sequence's activity is reduced in the cell sample after the contacting. In some embodiments, the present disclosure provides methods of identifying an anti-cancer agent, the method comprising contacting a candidate agent that binds to a cancer associated sequence selected from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a combination thereof with a cell sample, and assaying for the cancer associated sequence's activity or expression level, wherein the candidate antibody is identified as an anti-cancer agent if the cancer associated sequence's activity is reduced in the cell sample after the contacting.

In some embodiments, a method of screening drug candidates includes comparing the level of expression of the cancer-associated sequence in the absence of the drug candidate to the level of expression in the presence of the drug candidate.

Some embodiments are directed to a method of screening for a therapeutic agent capable of binding to a cancer-associated sequence (nucleic acid or protein), the method comprising combining the cancer-associated sequence and a candidate therapeutic agent, and determining the binding of the candidate agent to the cancer-associated sequence.

Further provided herein is a method for screening for a therapeutic agent capable of modulating the activity of a cancer-associated sequence. In some embodiments, the method comprises combining the cancer-associated sequence and a candidate therapeutic agent, and determining the effect of the candidate agent on the bioactivity of the cancer-associated sequence. An agent that modulates the bioactivity of a cancer associated sequence may be used as a therapeutic agent capable of modulating the activity of a cancer-associated sequence.

In certain embodiments the invention provides a method of screening for anticancer activity comprising: (a) contacting a cell that expresses a cancer associated gene selected from one or more cancer associated sequences disclosed infra, homologs thereof, combinations thereof, or fragments thereof with an anticancer drug candidate; (b) detecting an effect of the anticancer drug candidate on an expression of the cancer associated sequence in the cell (either at the nucleic acid or protein level); and (c) comparing the level of expression in the absence of the drug candidate to the level of expression in the presence of the drug candidate; wherein an effect on the expression of the cancer associate polynucleotide indicates that the candidate has anticancer activity. For example the drug candidate may lower the expression level of the cancer associated sequence in the cell.

In some embodiments, a method of evaluating the effect of a candidate cancer drug may comprise administering the drug to a patient and removing a cell sample from the patient. The expression profile of the cell is then determined. In some embodiments, the method may further comprise comparing the expression profile of the patient to an expression profile of a healthy individual. In some embodiments, the expression profile comprises measuring the expression of one or more or any combination thereof of the sequences disclosed herein. In some embodiments, where the expression profile of one or more or any combination thereof of the sequences disclosed herein is modified (increased or decreased) the candidate cancer drug is said to be effective.

In some embodiments, the invention provides a method of screening for anticancer activity comprising: (a) providing a cell that expresses a cancer associated gene that encodes a nucleic acid sequence selected from the group consisting of the cancer associated sequences shown Tables 1, 2 and 6, or fragment thereof, (b) contacting the cell, which can be derived from a cancer cell with an anticancer drug candidate; (c) monitoring an effect of the anticancer drug candidate on an expression of the cancer associated sequence in the cell sample, and optionally (d) comparing the level of expression in the absence of said drug candidate to the level of expression in the presence of the drug candidate.

Suitable drug candidates include, but are not limited to an inhibitor of transcription, a G-protein coupled receptor antagonist, a growth factor antagonist, a serine-threonine kinase antagonist, a tyrosine kinase antagonist. In some embodiments, where the candidate modulates the expression of the cancer associated sequence the candidate is said to have anticancer activity. In some embodiments, the anticancer activity is determined by measuring cell growth. In some embodiments, the candidate inhibits or retards cell growth and is said to have anticancer activity. In some embodiments, the candidate causes the cell to die, and thus, the candidate is said to have anticancer activity.

In some embodiments, the present invention provides a method of screening for activity against pancreatic cancer. In some embodiments, the method comprises contacting a cell that overexpresses a cancer associated gene which is complementary to a cancer associated sequence selected from cancer associated sequences disclosed infra, homologs thereof; combinations thereof, or fragments thereof with a pancreatic cancer drug candidate. In some embodiments, the method comprises detecting an effect of the pancreatic cancer drug candidate on an expression of the cancer associated polynucleotide in the cell or an effect on the cell's growth or viability. In some embodiments, the method comprises comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against a pancreatic cancer cell that overexpresses a cancer associated gene, wherein said gene comprises a sequence that is a sequence selected from sequences encoding the genes listed in tables 1, 2, 6 or fragments thereof, or complementary thereto, homologs thereof, combinations thereof; or fragments thereof. In some embodiments, the drug candidate may include, for example, a transcription inhibitor, a G-protein coupled receptor antagonist, a growth factor antagonist, a serine-threonine kinase antagonist, or a tyrosine kinase antagonist.

Methods of Identifying Pancreatic Cancer Markers

The pattern of gene expression in a particular living cell may be characteristic of its current state. Nearly all differences in the state or type of a cell are reflected in the differences in RNA levels of one or more genes. Comparing expression patterns of uncharacterized genes may provide clues to their function. High throughput analysis of expression of hundreds or thousands of genes can help in (a) identification of complex genetic diseases, (b) analysis of differential gene expression over time, between tissues and disease states, and (c) drug discovery and toxicology studies. Increase or decrease in the levels of expression of certain genes correlate with cancer biology. For example, oncogenes are positive regulators of tumorigenesis, while tumor suppressor genes are negative regulators of tumorigenesis. (Marshall, Cell, 64: 313-406 (1991); Weinberg, Science, 254: 1138-1146 (1991)). Accordingly, some embodiments herein provide for polynucleotide and polypeptide sequences involved in cancer and, in particular, in oncogenesis.

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes. Carcinogenesis is fundamentally driven by somatic cell evolution (i.e. mutation and natural selection of variants with progressive loss of growth control). The genes that serve as targets for these somatic mutations are classified as either protooncogenes or tumor suppressor genes, depending on whether their mutant phenotypes are dominant or recessive, respectively.

Some embodiments of the invention are directed to cancer associated sequences (“target markers”). Some embodiments are directed to methods of identifying novel target markers useful in the diagnosis and treatment of cancer wherein expression levels of mRNAs, miRNAs, proteins, or protein post translational modifications including but not limited to phosphorylation and sumoylation are compared between five categories of cell types: (1) immortal pluripotent stem cells (such as embryonic stem (“ES”) cells, induced pluripotent stem (“iPS”) cells, and germ-line cells such as embryonal carcinoma (“EC”) cells) or gonadal tissues; (2) ES, iPS, or EC-derived clonal embryonic progenitor (“EP”) cell lines, (3) nucleated blood cells including but not limited to CD34+ cells and CD 133+ cells; (4) normal mortal somatic adult-derived tissues and cultured cells including: skin fibroblasts, vascular endothelial cells, normal non-lymphoid and non-cancerous tissues, and the like, and (5) malignant cancer cells including cultured cancer cell lines or human tumor tissue. mRNAs, miRNAs, or proteins that are generally expressed (or not expressed) in categories 1, 3, and 5, or categories 1 and 5 but not expressed (or expressed) in categories 2 and 4 are candidate targets for cancer diagnosis and therapy. Some embodiments herein are directed to human applications, non-human veterinary applications, or a combination thereof.

In some embodiments, a method of identifying a target marker comprises the steps of: 1) obtaining a molecular profile of the mRNAs, miRNAs, proteins, or protein modifications of immortal pluripotent stein cells (such as embryonic stem (“ES”) cells, induced pluripotent stem (“iPS”) cells, and germ-line cells such as embryonal carcinoma (“EC”) cells); 2) ES, iPS, or EC-derived clonal embryonic progenitor (“EP”) cell lines malignant cancer cells including cultured cancer cell lines or human tumor tissues, and comparing those molecules to those present in mortal somatic cell types such as cultured clonal human embryonic progenitors, cultured somatic cells from fetal or adult sources, or normal tissue counterparts to malignant cancer cells. Target markers that are shared between pluripotent stem cells such as hES cells and malignant cancer cells, but are not present in a majority of somatic cell types may be candidate diagnostic markers and therapeutic targets.

Cancer associated sequences of embodiments herein are disclosed, for example, one or more of the genes disclosed in Tables 1, 2 and 6. These sequences were extracted from fold-change and filter analysis. Expression of cancer associated sequences in normal and pancreatic tumor tissues is disclosed infra.

Once expression is determined, the gene sequence results may be further filtered by considering fold-change in cancer cell lines vs. normal tissue; general specificity; secreted or not, level of expression in cancer cell lines; and signal to noise ratio.

It will be appreciated that there are various methods of obtaining expression data and uses of the expression data. For example, the expression data that can be used to detect or diagnose a subject with cancer can be obtained experimentally. In some embodiments, obtaining the expression data comprises obtaining the sample and processing the sample to experimentally determine the expression data. The expression data can comprise expression data for one or more of the cancer associated sequences described herein. The expression data can be experimentally determined by, for example, using a microarray or quantitative amplification method such as, but not limited to, those described herein. In some embodiments, obtaining expression data associated with a sample comprises receiving the expression data from a third party that has processed the sample to experimentally determine the expression data.

Detecting a level of expression or similar steps that are described herein may be done experimentally or provided by a third-party as is described herein. Therefore, for example, “detecting a level of expression” may refer to experimentally measuring the data and/or having the data provided by another party who has processed a sample to determine and detect a level of expression data.

The comparison of gene expression on an mRNA level using Illumina gene expression microarrays hybridized to RNA probe sequences may be used. For example samples may be prepared from diverse categories of cell types: 1) human embryonic stem (“ES”) cells, or gonadal tissues 2) ES, iPS, or EC-derived clonal embryonic progenitor (“EP”) cell lines, 3) nucleated blood cells including but not limited to CD34+ cells and CD133+ cells; 4) Normal mortal somatic adult-derived tissues and cultured cells including: skin fibroblasts, vascular endothelial cells, normal non-lymphoid and non-cancerous tissues, and the like, and 5) malignant cancer cells including cultured cancer cell lines or human tumor tissue and filters was performed to detect genes that are generally expressed (or not expressed) in categories 1, 3, and 5, or categories 1 and 5 but not expressed (or expressed) in categories 2 and 4. Therapies in these cancers based on this observation would be based on reducing the expression of the above referenced transcripts up-regulated in cancer, or otherwise reducing the expression of the gene products.

Techniques for Analyzing Samples

Any technique known in the art may be used to analyze a sample according to the methods disclosed infra such as methods of detecting or diagnosing cancer in a sample or identifying a new cancer associated sequence. Exemplary techniques are provided below.

Gene Expression Assays: Measurement of the gene expression levels may be performed by any known methods in the art, including but not limited to quantitative PCR, or microarray gene expression analysis, bead array gene expression analysis and Northern analysis. The gene expression levels may be represented as relative expression normalized to the ADPRT (Accession number NM_{—}001618.2), GAPD (Accession number NM_{—}002046.2), or other housekeeping genes known in the art. In the case of microarrayed probes of mRNA expression, the gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines.

RNA extraction: Cells of the present disclosure may be incubated with 0.05% trypsin and 0.5 mM EDTA, followed by collecting in DMEM (Gibco, Gaithersburg, Md.) with 0.5% BSA. Total RNA may be purified from cells using the RNeasy Mini kit (Qiagen, Hilden, Germany).

Isolation of total RNA and miRNA from cells: Total RNA or samples enriched for small RNA species may be isolated from cell cultures that undergo serum starvation prior to harvesting RNA to approximate cellular growth arrest observed in many mature tissues. Cellular growth arrest may be performed by changing to medium containing 0.5% serum for 5 days, with one medium change 2-3 days after the first addition of low serum medium. RNA may be harvested according to the vendor's instructions for Qiagen RNEasy kits to isolate total RNA or Ambion mirVana kits to isolate RNA enriched for small RNA species. The RNA concentrations may be determined by spectrophotometry and RNA quality may be determined by denaturing agarose gel electrophoresis to visualize 28S and 18S RNA. Samples with clearly visible 28S and 18S bands without signs of degradation and at a ratio of approximately 2:1, 28S: 18S may be used for subsequent miRNA analysis.

Assay for miRNA in samples isolated from human cells: The miRNAs may be quantitated using a Human Panel TaqMan MicroRNA Assay from Applied Biosystems, Inc. This is a two-step assay that uses stem-loop primers for reverse transcription (RT) followed by real-time TaqMan®. The assay includes two steps, reverse transcription (RT) and quantitative PCR. Real-time PCR may be performed on an Applied Biosystems 7500 Real-Time PCR System. The copy number per cell may be estimated based on the standard curve of synthetic mir-16 miRNA and assuming a total RNA mass of approximately 15 pg/cell.

The reverse transcription reaction may be performed using 1× cDNA archiving buffer, 3.35 units MMLV reverse transcriptase, 5 mM each dNTP, 1.3 units AB RNase inhibitor, 2.5 nM 330-plex reverse primer (RP), 3 ng of cellular RNA in a final volume of 5 μl. The reverse transcription reaction may be performed on a BioRad or MJ thermocycler with a cycling profile of 20° C. for 30 sec; 42° C. for 30 sec; 50° C. for 1 sec, for 60 cycles followed by one cycle of 85° C. for 5 min.

Real-Time PCR.

Two microlitres of 1:400 diluted Pre-PCR product may be used for a 20 ul reaction. All reactions may be duplicated. Because the method is very robust, duplicate samples may be sufficient and accurate enough to obtain values for miRNA expression levels. TaqMan universal PCR master mix of ABI may be used according to manufacturer's suggestion. Briefly, 1× TaqMan Universal Master Mix (ABI), 1 uM Forward Primer, 1 uM Universal Reverse Primer and 0.2 uM TaqMan Probe may be used for each real-time PCR. The conditions used may be as follows: 95° C. for 10 min, followed by 40 cycles at 95° C. for 15 s, and 60° C. for 1 min. All the reactions may be run on ABI Prism 7000 Sequence Detection System.

Microarray Hybridization and Data Processing.

cDNA samples and cellular total RNA (5 μg in each of eight individual tubes) may be subjected to the One-Cycle Target Labeling procedure for biotin labeling by in vitro transcription (IVT) (Affymetrix, Santa Clara, Calif.) or using the Illumina Total Prep RNA Labelling kit. For analysis on Affymetix gene chips, the cRNA may be subsequently fragmented and hybridized to the Human Genome U133 Plus 2.0 Array (Affymetrix) according to the manufacturer's instructions. The microarray image data may be processed with the GeneChip Scanner 3000 (Affymetrix) to generate CEL data. The CEL data may be then subjected to analysis with dChip software, which has the advantage of normalizing and processing multiple datasets simultaneously. Data obtained from the eight nonamplified controls from cells, from the eight independently amplified samples from the diluted cellular RNA, and from the amplified cDNA samples from 20 single cells may be normalized separately within the respective groups, according to the program's default setting. The model based expression indices (MBEI) may be calculated using the PM/MM difference mode with log-2 transformation of signal intensity and truncation of low values to zero. The absolute calls (Present, Marginal and Absent) may be calculated by the Affymetrix Microarray Software 5.0 (MAS 5.0) algorithm using the dChip default setting. The expression levels of only the Present probes may be considered for all quantitative analyses described below. The GEO accession number for the microarray data is GSE4309. For analysis on Illumina Human HT-12 v4 Expression Bead Chips, labeled cRNA may be hybridized according to the manufacturer's instructions.

Calculation of Coverage and Accuracy.

A true positive is defined as probes called Present in at least six of the eight nonamplified controls, and the true expression levels are defined as the log-averaged expression levels of the Present probes. The definition of coverage is (the number of truly positive probes detected in amplified samples)/(the number of truly positive probes). The definition of accuracy is (the number of truly positive probes detected in amplified samples)/(the number of probes detected in amplified samples). The expression levels of the amplified and nonamplified samples may be divided by the class interval of 20.5 (20, 20.5, 21, 21.5 . . . ), where accuracy and coverage are calculated. These expression level bins may be also used to analyze the frequency distribution of the detected probes.

Analysis of Gene Expression Profiles of Cells:

The unsupervised clustering and class neighbor analyses of the microarray data from cells may be performed using GenePattern software (http://www.broad.mit.edu/cancer/software/genepattern/), which performs the signal-to-noise ratio analysis/T-test in conjunction with the permutation test to preclude the contribution of any sample variability, including those from methodology and/or biopsy, at high confidence. The analyses may be conducted on the 14,128 probes for which at least 6 out of 20 single cells provided Present calls and at least 1 out of 20 samples provided expression levels >20 copies per cell. The expression levels calculated for probes with Absent/Marginal calls may be truncated to zero. To calculate relative gene expression levels, the Ct values obtained with Q-PCR analyses may be corrected using the efficiencies of the individual primer pairs quantified either with whole human genome (BD Biosciences) or plasmids that contain gene fragments. The relative expression levels may be further transformed into copy numbers with a calibration line calculated using the spike RNAs included in the reaction mixture (log₁₀[expression level]=1.05×log₁₀[copy number]+4.65). The Chi-square test for independence may be performed to evaluate the association of gene expressions with Gata4, which represents the difference between cluster 1 and cluster 2 determined by the unsupervised clustering and which is restricted to PE at later stages. The expression levels of individual genes measured with Q-PCR may be classified into three categories: high (>100 copies per cell), middle (10-100 copies per cell), and low (<10 copies per cell). The Chi-square and P-values for independence from Gata4 expression may be calculated based on this classification. Chi squared is defined as follows: χ2=ΣΣ(n fij−fi fj)²/n fi fj, where i and j represent expression level categories (high, middle or low) of the reference (Gata4) and the target gene, respectively; fi, f, and fij represent the observed frequency of categories i, j and ij, respectively; and n represents the sample number (n=24). The degrees of freedom may be defined as (r−1)×(c−1), where r and c represent available numbers of expression level categories of Gata4 and of the target gene, respectively.

Generating an Immune Response Against Pancreatic Cancer

In some embodiments, antigen presenting cells (APCs) may be used to activate T lymphocytes in vivo or ex vivo, to elicit an immune response against cells expressing a cancer associated sequence. APCs are highly specialized cells and may include, without limitation, macrophages, monocytes, and dendritic cells (DCs). APCs may process antigens and display their peptide fragments on the cell surface together with molecules required for lymphocyte activation. In some embodiments, the APCs may be dendritic cells. DCs may be classified into subgroups, including, e.g., follicular dendritic cells, Langerhans dendritic cells, and epidermal dendritic cells. In other embodiments the invention provides a method of eliciting an antibody response to one or more of the cancer associated sequences disclosed infra. The method may comprise administering a protein or a peptide fragment encoded by one or more of the cancer associated sequences disclosed infra to a subject.

Some embodiments are directed to the use of cancer associated polypeptides and polynucleotides encoding a cancer associated sequence, a fragment thereof, or a mutant thereof, and antigen presenting cells (such as, without limitation, dendritic cells), to elicit an immune response against cells expressing a cancer-associated polypeptide sequence, such as, without limitation, cancer cells, in a subject. In some embodiments, the method of eliciting an immune response against cells expressing a cancer associated sequence comprises (1) isolating a hematopoietic stem cell, (2) genetically modifying the cell to express a cancer associated sequence, (3) differentiating the cell into DCs; and (4) administering the DCs to the subject (e.g., human patient). In some embodiments, the method of eliciting an immune response includes (1) isolating DCs (or isolation and differentiation of DC precursor cells), (2) pulsing the cells with a cancer associated sequence, and; (3) administering the DCs to the subject. These approaches are discussed in greater detail, infra. In some embodiments, the pulsed or expressing DCs may be used to activate T lymphocytes ex vivo. These general techniques and variations thereof may be within the skill of those in the art (see, e.g., WO97/29182; WO 97/04802; WO 97/22349; WO 96/23060; WO 98/01538; Hsu et al., 1996, Nature Med. 2:52-58), and that still other variations may be discovered in the future. In some embodiments, the cancer associated sequence is contacted with a subject to stimulate an immune response. In some embodiments, the immune response is a therapeutic immune response so as to treat a subject as described infra. In some embodiments, the immune response is a prophylactic immune response. For example, the cancer associated sequence can be contacted with a subject under conditions effective to stimulate an immune response. The cancer associated sequence can be administered as, for example, a DNA molecule (e.g. DNA vaccine), RNA molecule, or polypeptide, or any combination thereof. Administering a sequence to stimulate an immune response was known, but the identity of which sequences to use was not known prior to the present disclosure. Any sequence or combination of sequences disclosed herein or a homolog thereof can be administered to a subject to stimulate an immune response.

In some embodiments, dendritic cell precursor cells are isolated for transduction with a cancer associated sequence, and induced to differentiate into dendritic cells. The genetically modified DCs express the cancer associated sequence, and may display peptide fragments on the cell surface.

In some embodiments, the cancer associated sequence expressed comprises a sequence of a naturally occurring protein. In some embodiments, the cancer associate sequence does not comprise a naturally occurring sequence. As already noted, fragments of naturally occurring proteins may be used; in addition, the expressed polypeptide may comprise mutations such as deletions, insertions, or amino acid substitutions when compared to a naturally occurring polypeptide, so long as at least one peptide epitope can be processed by the DC and presented on a MHC class I or II surface molecule. In some embodiments, it may be desirable to use sequences other than “wild type,” in order to, for example, increase antigenicity of the peptide or to increase peptide expression levels. In some embodiments, the introduced cancer associated sequences may encode variants such as polymorphic variants (e.g., a variant expressed by a particular human patient) or variants characteristic of a particular cancer (e.g., a cancer in a particular subject).

In some embodiments, a cancer associated sequence may be introduced (transduced) into DCs or stem cells in any of a variety of standard methods, including transfection, recombinant vaccinia viruses, adeno-associated viruses (AAVs), retroviruses, etc.

In some embodiments, the transformed DCs of the invention may be introduced into the subject (e.g., without limitation, a human patient) where the DCs may induce an immune response. Typically, the immune response includes a cytotoxic T-lymphocyte (CTL) response against target cells bearing antigenic peptides (e.g., in a MHC class I/peptide complex). These target cells are typically cancer cells.

In some embodiments, when the DCs are to be administered to a subject, they may preferably isolated from, or derived from precursor cells from, that subject (i.e., the DCs may administered to an autologous subject). However, the cells may be infused into HLA-matched allogeneic or HLA-mismatched allogeneic subject. In the latter case, immunosuppressive drugs may be administered to the subject.

In some embodiments, the cells may be administered in any suitable manner. In some embodiments, the cell may be administered with a pharmaceutically acceptable carrier (e.g., saline). In some embodiments, the cells may be administered through intravenous, intra-articular, intramuscular, intradermal, intraperitoneal, or subcutaneous routes. Administration (i.e., immunization) may be repeated at time intervals. Infusions of DC may be combined with administration of cytokines that act to maintain DC number and activity (e.g., GM-CSF, IL-12).

In some embodiments, the dose administered to a subject may be a dose sufficient to induce an immune response as detected by assays which measure T cell proliferation, T lymphocyte cytotoxicity, and/or effect a beneficial therapeutic response in the patient over time, e.g., to inhibit growth of cancer cells or result in reduction in the number of cancer cells or the size of a tumor.

In some embodiments, DCs are obtained (either from a patient or by in vitro differentiation of precursor cells) and pulsed with antigenic peptides having a cancer associated sequence. The pulsing results in the presentation of peptides onto the surface MI-IC molecules of the cells. The peptide/MHC complexes displayed on the cell surface may be capable of inducing a MHC-restricted cytotoxic T-lymphocyte response against target cells expressing cancer associated polypeptides (e.g., without limitations, cancer cells).

In some embodiments, cancer associated sequences used for pulsing may have at least about 6 or 8 amino acids and fewer than about 30 amino acids or fewer than about 50 amino acid residues in length. In some embodiments, an immunogenic peptide sequence may have from about 8 to about 12 amino acids. In some embodiments, a mixture of human protein fragments may be used; alternatively a particular peptide of defined sequence may be used. The peptide antigens may be produced by de novo peptide synthesis, enzymatic digestion of purified or recombinant human peptides, by purification of the peptide sequence from a natural source (e.g., a subject or tumor cells from a subject), or expression of a recombinant polynucleotide encoding a human peptide fragment.

In some embodiments, the amount of peptide used for pulsing DC may depend on the nature, size and purity of the peptide or polypeptide. In some embodiments, an amount of from about 0.05 ug/ml to about 1 mg/ml, from about 0.05 ug/ml to about 500 ug/ml, from about 0.05 ug/ml to about 250 ug/ml, from about 0.5 ug/ml to about 1 mg/ml, from about 0.5 ug/ml to about 500 ug/ml, from about 0.5 ug/ml to about 250 ug/ml, or from about 1 ug/ml to about 100 ug/ml of peptide may be used. After adding the peptide antigen(s) to the cultured DC, the cells may then be allowed sufficient time to take up and process the antigen and express antigen peptides on the cell surface in association with either class I or class II MHC. In some embodiments, the time to take up and process the antigen may be about 18 to about 30 hours, about 20 to about 30 hours, or about 24 hours.

Numerous examples of systems and methods for predicting peptide binding motifs for different WIC Class I and II molecules have been described. Such prediction could be used for predicting peptide motifs that will bind to the desired MI-IC Class I or II molecules. Examples of such methods, systems, and databases that those of ordinary skill in the art might consult for such purpose include:
I. Peptide Binding Motifs for MHC Class I and II Molecules; William E. Biddison, Roland Martin, Current Protocols in Immunology, Unit II (DOI: 10.1002/0471142735.ima01is36; Online Posting Date: May, 2001).

Reference 1 above, provides an overview of the use of peptide-binding motifs to predict interaction with a specific MHC class I or II allele, and gives examples for the use of MHC binding motifs to predict T-cell recognition.

Table 3 provides an exemplary result for a HLA peptide motif search at the NIH Center for Information Technology website, BioInformatics and Molecular Analysis Section.

TABLE 3
exemplary result for HLA peptide motif search

| | |
|---|---|
| User Parameter and Scoring Information: | Explicit number |
| Method selected to mimic the number of | |
| results | |
| Number of results requested | 20 |
| HLA molecule type selected | A_0201 |
| Length selected for subsequences to be | 9 |
| scored | |
| Echoing mode selected for input sequence | Y |
| Echoing format | Numbered lines |
| Length of user's input peptide sequence | 369 |
| Number of subsequence scores calculated | 361 |
| Number of top-scoring subsequences | 20 |
| reported back in scoring output table | |

| | | | Score (estimate of |
|---|---|---|---|
| | | | half time of |
| | | | disassociation of a |
| | Scoring Results | Subsequence residue | molecule containing |
| Rank | Start Position | listing | this subsequence |
| 1 | 310 | SLLKFLAKV | 2249.173 |
| | | (SEQ ID NO: 57) | |
| 2 | 183 | MLLVFGIDV | 1662.432 |
| | | (SEQ ID NO: 58) | |
| 3 | 137 | KVTDLVQFL | 339.313 |
| | | (SEQ ID NO: 59) | |
| 4 | 254 | GLYDGMMEHL | 315.870 |
| | | (SEQ ID NO: 60) | |
| 5 | 228 | ILILSIIFI | 224.357 |
| | | (SEQ ID NO: 61) | |
| 6 | 296 | FLWGPRAHA | 189.678 |
| | | (SEQ ID NO: 62) | |
| 7 | 245 | VIWEALNMM | 90.891 |
| | | (SEQ ID NO: 63) | |
| 8 | 308 | KMSILKFLA | 72.836 |
| | | (SEQ ID NO: 64) | |
| 9 | 166 | KNYEDHFPL | 37.140 |
| | | (SEQ ID NO: 65) | |
| 10 | 201 | FVLVTSLGL | 31.814 |
| | | (SEQ ID NO: 66) | |
| 11 | 174 | ILFSEASEC | 31.249 |
| | | (SEQ ID NO: 67) | |
| 12 | 213 | GMLSDVQSM | 30.534 |
| | | (SEQ ID NO: 68) | |
| 13 | 226 | TLILILSII | 16.725 |
| | | (SEQ ID NO: 69) | |
| 14 | 225 | GILILILSI | 12.208 |
| | | (SEQ ID NO: 70) | |
| 15 | 251 | NMMGLYDGM | 9.758 |
| | | (SEQ ID NO: 71) | |
| 16 | 88 | QIACSSPSV | 9.563 |
| | | (SEQ ID NO: 72) | |
| 17 | 66 | LIPSTPEEV | 7.966 |
| | | (SEQ ID NO: 73) | |
| 18 | 220 | SMPKTGILI | 7.535 |
| | | (SEQ ID NO: 74) | |
| 19 | 233 | IIFIEGYCT | 6.445 |
| | | (SEQ ID NO: 75) | |
| 20 | 247 | WEALNMGL | 4.395 |
| | | (SEQ ID NO:76) | |

One skilled in the art of peptide-based vaccination may determine which peptides would work best in individuals based on their HLA alleles (e.g., due to “MHC restriction”). Different HLA alleles will bind particular peptide motifs (usually 2 or 3 highly conserved positions out of 8-10) with different energies which can be predicted theoretically or measured as dissociation rates. Thus, a skilled artisan may be able to tailor the peptides to a subject's HLA profile.

In some embodiments, the present disclosure provides methods of eliciting an immune response against cells expressing a cancer associated sequence comprising contacting a subject with a cancer associated sequence under conditions effective to elicit an immune response in the subject, wherein said cancer associated sequence comprises a sequence or fragment thereof a gene selected from one or more of the cancer associated sequences provided infra.

Transfecting Cells with Cancer Associated Sequences

Cells may be transfected with one or more of the cancer associated sequences disclosed infra. Transfected cells may be useful in screening assays, diagnosis and detection assays. Transfected cells expressing one or more cancer associated sequence disclosed herein may be used to obtain isolated nucleic acids encoding cancer associated sequences and/or isolated proteins or peptide fragments encoded by one or more cancer associated sequences.

Electroporation may be used to introduce the cancer associated nucleic acids described herein into mammalian cells (Neumann, E. et al. (1982) EMBO J. 1, 841-845), plant and bacterial cells, and may also be used to introduce proteins (Marrero, M. B. et al. (1995) J. Biol. Chem. 270, 15734-15738; Nolkrantz, K. et al. (2002) Anal. Chem. 74, 4300-4305; Rui, M. et al, (2002) Life Sci. 71, 1771-1778). Cells (such as the cells of this invention) suspended in a buffered solution of the purified protein of interest are placed in a pulsed electrical field. Briefly, high-voltage electric pulses result in the formation of small (nanometer-sized) pores in the cell membrane. Proteins enter the cell via these small pores or during the process of membrane reorganization as the pores close and the cell returns to its normal state. The efficiency of delivery may be dependent upon the strength of the applied electrical field, the length of the pulses, temperature and the composition of the buffered medium. Electroporation is successful with a variety of cell types, even some cell lines that are resistant to other delivery methods, although the overall efficiency is often quite low. Some cell lines may remain refractory even to electroporation unless partially activated.

Microinjection may be used to introduce femtoliter volumes of DNA directly into the nucleus of a cell (Capecchi, M. R. (1980) Cell 22, 470-488) where it can be integrated directly into the host cell genome, thus creating an established cell line bearing the sequence of interest. Proteins such as antibodies (Abarzua, P. et al. (1995) Cancer Res. 55, 3490-3494; Theiss, C. and Meller, K. (2002) Exp. Cell Res. 281, 197-204) and mutant proteins (Naryanan, A. et al. (2003) J. Cell Sci. 116, 177-186) can also be directly delivered into cells via microinjection to determine their effects on cellular processes firsthand. Microinjection has the advantage of introducing macromolecules directly into the cell, thereby bypassing exposure to potentially undesirable cellular compartments such as low-pH endosomes.

Several proteins and small peptides have the ability to transduce or travel through biological membranes independent of classical receptor-mediated or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. In some embodiments, protein transduction domains (PTDs) from these proteins may be fused to other macromolecules, peptides or proteins such as, without limitation, a cancer associated polypeptide to successfully transport the polypeptide into a cell (Schwarze, S. R. et al. (2000) Trends Cell Biol. 10, 290-295). Exemplary advantages of using fusions of these transduction domains is that protein entry is rapid, concentration-dependent and appears to work with difficult cell types (Fenton, M. et al. (1998) J. Immunol. Methods 212, 41-48).

In some embodiments, liposomes may be used as vehicles to deliver oligonucleotides, DNA (gene) constructs and small drug molecules into cells (Zabner, J. et al. (1995) J. Biol. Chem. 270, 18997-19097; Feigner, P. L. et al. (1987) Proc. Natl. Acad. Sci. USA 84, 7413-7417). Certain lipids, when placed in an aqueous solution and sonicated, form closed vesicles consisting of a circularized lipid bilayer surrounding an aqueous compartment. The vesicles or liposomes of embodiments herein may be formed in a solution containing the molecule to be delivered. In addition to encapsulating DNA in an aqueous solution, cationic liposomes may spontaneously and efficiently form complexes with DNA, with the positively charged head groups on the lipids interacting with the negatively charged backbone of the DNA. The exact composition and/or mixture of cationic lipids used can be altered, depending upon the macromolecule of interest and the cell type used (Feigner, J. H. et al. (1994) J. Biol. Chem. 269, 2550-2561). The cationic liposome strategy has also been applied successfully to protein delivery (Zelphati, O. et al. (2001) J. Biol. Chem. 276, 35103-35110). Because proteins are more heterogeneous than DNA, the physical characteristics of the protein, such as its charge and hydrophobicity, may influence the extent of its interaction with the cationic lipids.

Pharmaceutical Compositions and Modes of Administration

Modes of administration for a therapeutic (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of therapeutic to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the therapeutic of the present disclosure and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present disclosure. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions of the present disclosure can be formulated for parenteral administration by injection, e.g., by bolus injection or continuous infusion. The compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compositions can be formulated readily by combining the therapeutic with pharmaceutically acceptable carriers well known in the art. Such carriers enable the therapeutic of the invention to be formulated as tablets, pills, (knees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active therapeutic doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active therapeutic can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the pharmaceutical compositions can take the form of, e.g., tablets or lozenges formulated in a conventional manner.

For administration by inhalation, the therapeutic for use according to the present disclosure is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the therapeutic and a suitable powder base such as lactose or starch.

The compositions of the present disclosure can also be formulated in rectal compositions such as suppositories or retention enemas, e.g., containing conventional suppository bases such as cocoa butter or other glycerides.

In addition to the formulations described previously, the therapeutic of the present disclosure can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present disclosure, for example, can be applied to a plaster, or can be applied by transdermal, therapeutic systems that are consequently supplied to the organism.

Pharmaceutical compositions can include suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compositions of the present disclosure can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component may include one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium steatylfumarate, fatty acid, fatty alcohol, fatty acid ester, glycerylbehenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

Kits

Also provided by the invention are kits and systems for practicing the subject methods, as described above, such components configured to diagnose cancer in a subject, treat cancer in a subject, detect cancer in a sample, or perform basic research experiments on cancer cells (e.g., either derived directly from a subject, grown in vitro or ex vivo, or from an animal model of cancer. The various components of the kits may be present in separate containers or certain compatible components may be pre-combined into a single container, as desired.

In some embodiments, the invention provides a kit for diagnosing the presence of cancer in a test sample, said kit comprising at least one polynucleotide that selectively hybridizes to a cancer associated polynucleotide sequence encoding one or more genes provided in Tables 1, 2 and 6, or its complement. In another embodiment the invention provides an electronic library comprising a cancer associated polynucleotide, a cancer associated polypeptide, or fragment thereof, disclosed infra. In some embodiments the kit may include one or more capture reagents or specific binding partners of one or more cancer associated sequences disclosed infra.

The subject systems and kits may also include one or more other reagents for performing any of the subject methods. The reagents may include one or more matrices, solvents, sample preparation reagents, buffers, desalting reagents, enzymatic reagents, denaturing reagents, probes, polynucleotides, vectors (e.g., plasmid or viral vectors), etc., where calibration standards such as positive and negative controls may be provided as well. As such, the kits may include one or more containers such as vials or bottles, with each container containing a separate component for carrying out a sample processing or preparing step and/or for carrying out one or more steps for producing a normalized sample according to the present disclosure.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control samples and reagents, e.g., two or more control samples for use in testing the kit.

### Additional Embodiments of the Invention

In some embodiments, the methods comprise targeting a marker that is expressed at abnormal levels in pancreatic tumor tissue in comparison to normal somatic tissue. In some embodiments, the marker may comprise a sequence disclosed herein or in Table 1 or Table 4, a complement thereof, or a combination thereof. In some embodiments, the marker may be selected from a sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a homolog thereof, a fragment thereof, a complement thereof or a combination thereof. Some embodiments are directed to methods of treating pancreatic cancer comprising administering a composition including a therapeutic that affects the expression, abundance or activity of a target marker. In some embodiments, the target marker may include a sequence described herein or in Table 1, 2 OR 6, a complement thereof, or any combination thereof.

Some embodiments provide methods of detecting pancreatic cancer comprising detecting a level of a target marker associated with the cancer. In some embodiments, the target marker may include a sequence described herein or in Table 1 or Table 4, a complement thereof or any combination thereof. In some embodiments, the marker may be selected from a sequence selected from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6 orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a fragment thereof, a complement thereof or a combination thereof. In some embodiments, the marker may be selected from one of the genes provided in tables 1, 2 and 6, a fragment thereof, a complement thereof, or a combination thereof.

In some embodiments, a method of detecting pancreatic adenocarcinoma may comprise detecting a level of a target marker selected from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof.

In some embodiments, a method of detecting pancreatic neuroendocrine carcinoma may comprise detecting a level of a target marker selected from WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof.

Some embodiments herein provide antigens (i.e. cancer-associated polypeptides) associated with pancreatic cancer as targets for diagnostic and/or therapeutic antibodies. In some embodiments, the antigen may be selected from a sequence selected from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a fragment thereof, a complement thereof or a combination thereof. In some embodiments, these antigens may be useful for drug discovery (e.g., small molecules) and for further characterization of cellular regulation, growth, and differentiation.

In some embodiments, antigens associated with pancreatic adenocarcinoma may comprise a sequence selected from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof. In some embodiments, the sequence may be selected from SEQ ID NOs: 1-30, a fragment thereof, a complement thereof, or a combination thereof.

In some embodiments, antigens associated with pancreatic neuroendocrine carcinoma may comprise a sequence selected from WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof.

Some embodiments describe a method of diagnosing pancreatic cancer in a subject, the method comprising: (a) determining the expression of one or more genes or gene products or homologs thereof; and (b) comparing the expression of the one or more nucleic acid sequences from a second normal sample from the first subject or a second unaffected subject, wherein a difference in the expression indicates that the first subject has pancreatic cancer, wherein the gene or the gene product is referred to as a gene selected from: PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a gene described in Table 1, 2 or 6 (with sequences incorporated by reference via the accession number), a fragment thereof; a complement thereof or a combination thereof.

In some embodiments, a method of diagnosing pancreatic adenocarcinoma in a subject may comprise (a) determining the expression of one or more genes or gene products or homologs thereof; and (b) comparing the expression of the one or more nucleic acid sequences from a second normal sample from the first subject or a second unaffected subject, wherein a difference in the expression indicates that the first subject has pancreatic cancer, wherein the gene product is encoded by or the gene comprises a sequence selected from: PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof.

In some embodiments, a method of diagnosing pancreatic neuroendocrine carcinoma in a subject may comprise (a) determining the expression of one or more genes or gene products or homologs thereof; and (b) comparing the expression of the one or more nucleic acid sequences from a second normal sample from the first subject or a second unaffected subject, wherein a difference in the expression indicates that the first subject has pancreatic cancer, wherein the gene product is encoded by or the gene comprises a sequence selected from: WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof.

Some embodiments describe a method of eliciting an immune response against cells expressing a cancer associated sequence comprising contacting a subject with a cancer associated sequence under conditions effective to elicit an immune response in the subject, wherein the cancer associated sequence comprises a sequence or fragment thereof selected from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a sequence genes disclosed in table 1, 2 and 6 (including but not limited to the sequences described in the accession files), or a combination thereof.

Some embodiments describe a method of detecting pancreatic cancer in a test sample, comprising: (i) detecting a level of activity of at least one polypeptide that is a gene product; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of pancreatic cancer in the test sample, wherein the gene product is a product of a gene selected from: PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a sequence described in Table 1, 2 and 6, or a combination thereof.

In some embodiments, a method of detecting pancreatic adenocarcinoma in a subject may comprise (i) detecting a level of activity of at least one polypeptide that is a gene product; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of pancreatic cancer in the test sample, wherein the gene product is a product of a gene selected from: PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof.

In some embodiments, a method of detecting pancreatic neuroendocrine carcinoma in a subject may comprise ((i) detecting a level of activity of at least one polypeptide that is a gene product; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of pancreatic cancer in the test sample, wherein the gene product is a product of a gene selected from: WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof.

Some embodiments herein are directed to a method of treating pancreatic cancer in a subject, the method comprising administering to a subject in need thereof a therapeutic agent modulating the activity of a cancer associated protein, wherein the cancer associated protein is encoded by a nucleic acid comprising a nucleic acid sequence selected from a sequence described herein or in Table 1 or Table 4, homologs thereof, combinations thereof, or a fragment thereof. In some embodiments, the therapeutic agent binds to the cancer associated protein. In some embodiments, the therapeutic agent is an antibody. In some embodiments, the antibody may be a monoclonal antibody or a polyclonal antibody. In some embodiments, the antibody is a humanized or human antibody. In some embodiments, a method of treating pancreatic cancer may comprise gene knockdown of a gene selected from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a sequence described in Table 1 or Table 4, or a combination thereof. In some embodiments, a method of treating pancreatic cancer may comprise treating cells to knockdown or inhibit expression of a gene encoding an mRNA of PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a sequence described in Table 1 or Table 4, a fragment thereof, a complement thereof, or a combination thereof. In some embodiments, the cancer is pancreatic cancer. In some embodiments, the pancreatic cancer is selected from carcinoma, any malignant pancreatic neoplasm, ductal adenocarcinoma, cholangiocarcinoma, muinous carcinoma, adenosquamous carcinoma, signet ring cell carcinoma, hepatoid carcinomas, colloid carcinomas, undifferentiated carcinomas, pancreatic cystic neoplasms, islet cell tumors, pancreatic endocrine tumors, pancreatic neuroendocrine carcinoma, extrapulmonary small cell cancer, undifferentiated carcinoma or a combination thereof. The methods disclosed herein may also be used for diagnosis and treatment of other cancers and other conditions in which cells have become immortalized.

In some embodiments, a method of treating pancreatic adenocarcinoma in a subject may comprise gene knockdown of a gene selected from PPY, CHGB, ACE2, COL10A1, PRSS7, MEP1B, MMP12, MMP1, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof.

In some embodiments, a method of treating pancreatic neuroendocrine carcinoma in a subject may comprise gene knockdown of a gene selected from WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof.

In some embodiments, a method of diagnosing a subject with pancreatic cancer comprises obtaining a sample and detecting the presence of a cancer associated sequence selected from a sequence described herein or in Table 1, 2 and 6, a fragment thereof or a complement thereof wherein the presence of the cancer associated sequence indicates the subject has pancreatic cancer or a sequence that specifically hybridizes with a gene selected from the group of PPY, CHGB, ACE2, COL10A1, PRSS7, MEP1B, MMP12, MMP11, TM4SF2O, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a sequence described in Table 1 or Table 4 (sequence incorporated by reference via the accession number), a fragment thereof, a complement thereof; or a combination thereof. In some embodiments, detecting the presence of a cancer associated sequence comprises contacting the sample with an antibody or other type of capture reagent that specifically binds to the cancer associated sequence's protein and detecting the presence or absence of the binding to the cancer associated sequence's protein in the sample. In some embodiments, the pancreatic cancer is selected from carcinoma, any malignant pancreatic neoplasm, ductal adenocarcinoma, cholangiocarcinoma, muinous carcinoma, adenosquamous carcinoma, signet ring cell carcinoma, hepatoid carcinomas, colloid carcinomas, undifferentiated carcinomas, pancreatic cystic neoplasms, islet cell tumors, pancreatic endocrine tumors, pancreatic neuroendocrine carcinoma, extrapulmonary small cell cancer, undifferentiated carcinoma or a combination thereof. The methods disclosed herein may also be used for diagnosis and treatment of other conditions in which cells have become immortalized.

In some embodiments, a method of diagnosing pancreatic adenocarcinoma in a subject may comprise obtaining a sample and detecting the presence of a cancer associated sequence selected from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof. In some embodiments, the cancer associated sequence may be selected from SEQ ID NOs: 1-30, a fragment thereof; a complement thereof, or a combination thereof. In some embodiments, detecting the presence of a cancer associated sequence comprises contacting the sample with an antibody or other type of capture reagent that specifically binds to the cancer associated sequence's protein and detecting the presence or absence of the binding to the cancer associated sequence's protein in the sample.

In some embodiments, a method of diagnosing pancreatic neuroendocrine carcinoma in a subject may comprise obtaining a sample and detecting the presence of a cancer associated sequence selected from WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof. In some embodiments, detecting the presence of a cancer associated sequence comprises contacting the sample with an antibody or other type of capture reagent that specifically binds to the cancer associated sequence's protein and detecting the presence or absence of the binding to the cancer associated sequence's protein in the sample.

In some embodiments, the present invention provides methods of treating pancreatic cancer in a subject, the method comprising administering to a subject in need thereof a therapeutic agent that modulates the activity of PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a sequence described in Table 1 or Table 4 (sequence incorporated by reference via the accession number), a fragment thereof, a complement thereof, or a combination thereof, wherein the therapeutic agent treats the cancer in the subject. In some embodiments, the pancreatic cancer is selected from carcinoma, any malignant pancreatic neoplasm, ductal adenocarcinoma, cholangiocarcinoma, muinous carcinoma, adenosquamous carcinoma, signet ring cell carcinoma, hepatoid carcinomas, colloid carcinomas, undifferentiated carcinomas, pancreatic cystic neoplasms, islet cell tumors, pancreatic endocrine tumors, pancreatic neuroendocrine carcinoma, extrapulmonary small cell cancer, undifferentiated carcinoma or a combination thereof. The methods disclosed herein may also be used for treatment of other cancers and other conditions in which cells have become immortalized.

In some embodiments, a method of treating pancreatic adenocarcinoma in a subject may comprise administering to a subject in need thereof a therapeutic agent that modulates the activity of PPY, CHGB, ACE2, COL10A I, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof, wherein the therapeutic agent treats the pancreatic adenocarcinoma in the subject.

In some embodiments, a method of treating pancreatic neuroendocrine carcinoma in a subject may comprise administering to a subject in need thereof a therapeutic agent that modulates the activity of WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof; a complement thereof or a combination thereof, wherein the therapeutic agent treats the pancreatic neuroendocrine carcinoma in the subject.

In some embodiments, the present invention provides methods of diagnosing pancreatic cancer in a subject, the method comprising determining the expression of a gene disclosed in Table 1 or 4 or a gene selected from PPY, CHGB, ACE2, COL10A1, PRSS7, MEP1B, MMP12, MMP11, TM4SF2O, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a fragment thereof, a complement thereof, or a combination thereof from a sample; and diagnosing pancreatic cancer in the subject based on the expression, wherein the subject is diagnosed as having pancreatic cancer if the gene is overexpressed.

In some embodiments, a method of diagnosing pancreatic adenocarcinoma in a subject may comprise determining the expression of a gene selected from PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof; a complement thereof or a combination thereof; and diagnosing pancreatic adenocarcinoma in the subject based on the expression, wherein the subject is diagnosed as having pancreatic adenocarcinoma if the gene is overexpressed.

In some embodiments, a method of diagnosing pancreatic neuroendocrine carcinoma in a subject may comprise determining the expression of a gene selected from WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof; and diagnosing pancreatic neuroendocrine carcinoma in the subject based on the expression, wherein the subject is diagnosed as having pancreatic neuroendocrine carcinoma if the gene is overexpressed.

In some embodiments, the present invention provides methods of detecting pancreatic cancer in a test sample, the method comprising: (i) detecting a level of an antibody, wherein the antibody binds to an antigenic polypeptide encoded by a nucleic acid sequence comprising a sequence encoding PPY, CHGB, ACE2, COL10A1, PRSS7, MEP1B, MMP12, MMP1I, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a sequence described in Table 1 or Table 4 (sequence incorporated by reference via the accession number), a homolog thereof, a fragment thereof, a complement thereof, or a combination thereof; and (ii) comparing the level of the antibody in the test sample with a level of the antibody in a control sample, wherein an altered level of antibody in the test sample relative to the level of antibody in the control sample is indicative of the presence of pancreatic cancer in the test sample.

In some embodiments, a method of detecting pancreatic adenocarcinoma in a test sample may comprise (i) detecting a level of an antibody, wherein the antibody binds to an antigenic polypeptide encoded by a nucleic acid sequence comprising a sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof; and (ii) comparing the level of the antibody in the test sample with a level of the antibody in a control sample, wherein an altered level of antibody in the test sample relative to the level of antibody in the control sample is indicative of the presence of pancreatic adenocarcinoma in the test sample.

In some embodiments, a method of detecting pancreatic neuroendocrine carcinoma in a test sample may comprise (i) detecting a level of an antibody, wherein the antibody binds to an antigenic polypeptide encoded by a nucleic acid sequence comprising a sequence encoding WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof; and (ii) comparing the level of the antibody in the test sample with a level of the antibody in a control sample, wherein an altered level of antibody in the test sample relative to the level of antibody in the control sample is indicative of the presence of pancreatic neuroendocrine carcinoma in the test sample.

In some embodiments, the present invention provides methods of detecting pancreatic cancer in a test sample, comprising: (i) detecting a level of activity of at least one polypeptide that is encoded by a nucleic acid comprising a nucleic acid sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, a sequence described in Table 1 or Table 4 (sequence incorporated by reference via the accession number), a homolog thereof, a fragment thereof, a complement thereof, or a combination thereof; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of pancreatic cancer in the test sample.

In some embodiments, a method of detecting pancreatic adenocarcinoma in a test sample may comprise (i) detecting a level of activity of at least one polypeptide that is encoded by a nucleic acid comprising a nucleic acid sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of pancreatic adenocarcinoma in the test sample.

In some embodiments, a method of detecting pancreatic neuroendocrine carcinoma in a test sample may comprise (i) detecting a level of activity of at least one polypeptide that is encoded by a nucleic acid comprising a nucleic acid sequence encoding WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of pancreatic neuroendocrine carcinoma in the test sample.

In some embodiments, the present invention provides methods of detecting pancreatic cancer in a test sample, the method comprising: (1) detecting a level of expression of at least one polypeptide that is encoded by a nucleic acid comprising a nucleic acid sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a sequence disclosed in Table 1 or Table 4, homologs thereof, combinations thereof, or a fragment thereof; and (ii) comparing the level of expression of the polypeptide in the test sample with a level of expression of polypeptide in a normal sample, wherein an altered level of expression of the polypeptide in the test sample relative to the level of polypeptide expression in the normal sample is indicative of the presence of pancreatic cancer in the test sample.

In some embodiments, a method of detecting pancreatic adenocarcinoma in a test sample may comprise (i) detecting a level of expression of at least one polypeptide that is encoded by a nucleic acid comprising a nucleic acid sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof; and (ii) comparing the level of expression of the polypeptide in the test sample with a level of expression of polypeptide in a normal sample, wherein an altered level of expression of the polypeptide in the test sample relative to the level of polypeptide expression in the normal sample is indicative of the presence of pancreatic adenocarcinoma in the test sample.

In some embodiments, a method of detecting pancreatic neuroendocrine carcinoma in a test sample may comprise (1) detecting a level of expression of at least one polypeptide that is encoded by a nucleic acid comprising a nucleic acid sequence encoding WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof; and (ii) comparing the level of expression of the polypeptide in the test sample with a level of expression of polypeptide in a normal sample, wherein an altered level of expression of the polypeptide in the test sample relative to the level of polypeptide expression in the normal sample is indicative of the presence of pancreatic neuroendocrine carcinoma in the test sample.

In some embodiments, the present invention provides methods of detecting pancreatic cancer in a test sample, the method comprising: (i) detecting a level of expression of a nucleic acid sequence comprising a nucleic acid sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a sequence disclosed in Table 1, 2 and 6, homologs thereof, mutant nucleic acids thereof, combinations thereof, or a fragment thereof; and (ii) comparing the level of expression of the nucleic acid sequence in the test sample with a level of expression of nucleic acid sequence in a normal sample, wherein an altered level of expression of the nucleic acid sequence in the test sample relative to the level of nucleic acid sequence expression in the normal sample is indicative of the presence of pancreatic cancer in the test sample.

In some embodiments, a method of detecting pancreatic adenocarcinoma in a test sample may comprise (i) detecting a level of expression of a nucleic acid sequence comprising a nucleic acid sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof; and (ii) comparing the level of expression of the nucleic acid sequence in the test sample with a level of expression of nucleic acid sequence in a normal sample, wherein an altered level of expression of the nucleic acid sequence in the test sample relative to the level of nucleic acid sequence expression in the normal sample is indicative of the presence of pancreatic adenocarcinoma in the test sample.

In some embodiments, a method of detecting pancreatic neuroendocrine carcinoma in a test sample may comprise (i) detecting a level of expression of a nucleic acid sequence comprising a nucleic acid sequence encoding WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof; and (ii) comparing the level of expression of the nucleic acid sequence in the test sample with a level of expression of nucleic acid sequence in a normal sample, wherein an altered level of expression of the nucleic acid sequence in the test sample relative to the level of nucleic acid sequence expression in the normal sample is indicative of the presence of pancreatic neuroendocrine carcinoma in the test sample.

In some embodiments, the present invention provides methods of screening for activity against pancreatic cancer, the method comprising: (a) contacting a cell that expresses a cancer associated gene comprising a sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a sequence disclosed in Table 1 or Table 4, a complement thereof, homologs thereof, combinations thereof, or fragments thereof with a cancer drug candidate; (b) detecting an effect of the cancer drug candidate on an expression of the cancer associated polynucleotide in the cell; and (c) comparing the level of expression in the absence of the drug candidate to the level of expression in the presence of the drug candidate; wherein an effect on the expression of the cancer associate polynucleotide indicates that the candidate has activity against pancreatic cancer.

In some embodiments, a method of screening for activity against pancreatic adenocarcinoma may comprise (a) contacting a cell that expresses a cancer associated gene comprising a sequence encoding PPY, CHGB, ACE2, COL10A1, PRSS7, MEP1B, MMP12, MMP1I, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof; (b) detecting an effect of the cancer drug candidate on an expression of the cancer associated polynucleotide in the cell; and (c) comparing the level of expression in the absence of the drug candidate to the level of expression in the presence of the drug candidate; wherein an effect on the expression of the cancer associate polynucleotide indicates that the candidate has activity against pancreatic adenocarcinoma

In some embodiments, a method of screening for activity against pancreatic neuroendocrine carcinoma may comprise (a) contacting a cell that expresses a cancer associated gene comprising a sequence encoding WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof; (b) detecting an effect of the cancer drug candidate on an expression of the cancer associated polynucleotide in the cell; and (c) comparing the level of expression in the absence of the drug candidate to the level of expression in the presence of the drug candidate; wherein an effect on the expression of the cancer associate polynucleotide indicates that the candidate has activity against pancreatic neuroendocrine carcinoma.

In some embodiments, the present invention provides methods of screening for activity against pancreatic cancer, the method comprising: (a) contacting a cell that overexpresses a cancer associated gene comprising a sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a sequence disclosed in Table 1 or Table 4, a complement thereof, homologs thereof, combinations thereof, or fragments thereof with a cancer drug candidate; (b) detecting an effect of the cancer drug candidate on an expression of the cancer associated polynucleotide in the cell or an effect on cell growth or viability; and (e) comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against the pancreatic cancer cell that overexpresses a cancer associated gene comprising a sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a sequence disclosed in Table 1 or Table 4, a complement thereof, homologs thereof, combinations thereof; or fragments thereof.

In some embodiments, a method of screening for activity against pancreatic adenocarcinoma (a) contacting a cell that overexpresses a cancer associated gene comprising a sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof with a cancer drug candidate; (b) detecting an effect of the cancer drug candidate on an expression of the cancer associated polynucleotide in the cell or an effect on cell growth or viability; and (c) comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against the pancreatic cancer cell that overexpresses a cancer associated gene comprising a sequence encoding PPY, CHGB, ACE2, COL10A1, PRSS7, MEP1B, MMP12, MMP11, TM4SF2O, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof

In some embodiments, a method of screening for activity against pancreatic neuroendocrine carcinoma may comprise (a) contacting a cell that overexpresses a cancer associated gene comprising a sequence encoding WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof, (b) detecting an effect of the cancer drug candidate on an expression of the cancer associated polynucleotide in the cell or an effect on cell growth or viability; and (c) comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against the pancreatic cancer cell that overexpresses a cancer associated gene comprising a sequence encoding WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof.

In some embodiments, the present invention provides methods of diagnosing pancreatic cancer in a subject, the method comprising: a) determining the expression of one or more nucleic acid sequences, wherein the one or more nucleic acid sequences comprises a sequence encoding PPY, CHGB, ACE2, COL10A1, PRSS7, MEP1B, MMP12, MMP11, TM4SF2O, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a sequence disclosed in Table 1 or Table 4, homologs thereof, combinations thereof, or fragments thereof in a first sample of a first subject; and b) comparing the expression of the one or more nucleic acid sequences from a second normal sample from the first subject or a second unaffected subject, wherein a difference in the expression of a sequence disclosed herein or in Table 1 or Table 4 indicates that the first subject has pancreatic cancer.

In some embodiments, a method of diagnosing pancreatic adenocarcinoma in a subject may comprise a) determining the expression of one or more nucleic acid sequences from a first sample, wherein the one or more nucleic acid sequences comprises a sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof; and b) comparing the expression of the one or more nucleic acid sequences from a second normal sample from the first subject or a second unaffected subject, wherein a difference in the expression of the sequence from the first sample indicates that the rust subject has pancreatic adenocarcinoma.

In some embodiments, a method of diagnosing pancreatic neuroendocrine carcinoma in a subject may comprise a) determining the expression of one or more nucleic acid sequences from a first sample, wherein the one or more nucleic acid sequences comprises a sequence encoding WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof; and b) comparing the expression of the one or more nucleic acid sequences from a second normal sample from the first subject or a second unaffected subject, wherein a difference in the expression of the sequence from the first sample indicates that the first subject has pancreatic adenocarcinoma.

In some embodiments, the present invention provides methods of detecting cancer in a test sample, comprising: (i) detecting a level of activity of at least one polypeptide; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample, wherein the polypeptide is a gene product of a sequence disclosed in Table 1 or 4 or is a gene product of PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a sequence disclosed in Table 1, 2 and 6, homologs thereof, combinations thereof, or fragments thereof.

In some embodiments, a method of detecting pancreatic adenocarcinoma in a test sample may comprise (i) detecting a level of activity of at least one polypeptide; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample, wherein the polypeptide is a gene product of PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof.

In some embodiments, a method of detecting pancreatic neuroendocrine carcinoma in a test sample may comprise (I) detecting a level of activity of at least one polypeptide; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample, wherein the polypeptide is a gene product of WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof.

In some embodiments, the present invention provides methods of diagnosing pancreatic cancer in a subject, the method comprising: obtaining one or more gene expression results for one or more sequences, wherein the one or more sequences comprises a sequence encoding PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a sequence disclosed in Table 1 or Table 4, homologs thereof, combinations thereof, or fragments thereof, from a sample derived from a subject; and diagnosing cancer in the subject based on the one or more gene expression results, wherein the subject is diagnosed as having cancer if one or more genes is overexpressed.

In some embodiments, a method of diagnosing pancreatic adenocarcinoma in a subject may comprise obtaining one or more gene expression results for one or more sequences, wherein the one or more sequences comprises a sequence encoding: PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof; and diagnosing pancreatic adenocarcinoma in the subject based on the one or more gene expression results, wherein the subject is diagnosed as having cancer if one or more genes is overexpressed.

In some embodiments, a method of diagnosing pancreatic neuroendocrine carcinoma in a subject may comprise obtaining one or more gene expression results for one or more sequences, wherein the one or more sequences comprises a sequence encoding: WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof; and diagnosing pancreatic adenocarcinoma in the subject based on the one or more gene expression results, wherein the subject is diagnosed as having cancer if one or more genes is overexpressed.

In some embodiments, the present invention provides methods of diagnosing a subject with pancreatic cancer or as a person suspected of having pancreatic cancer by determining the amount of protein in a subject of PPY, CHGB, ACE2, COL10A1, PRSS7, MEP1B, MMP12, MMP11, TM4SF2O, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a protein product of a sequence disclosed in Table 1, 2 and 6, homologs thereof, combinations thereof, or fragments thereof. The amount of protein can be determined in a sample, such as but not limited to, serum, blood, or urine.

In some embodiments, a method of diagnosing pancreatic adenocarcinoma or as a person suspected of having pancreatic cancer comprises determining the amount of protein in a subject of encoded by a sequence selected from: PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6ORF222, RBP2, TNFRSF6B, TNFRSF6B, CST1, CTSE, SI, LUM, C5ORF46, MMP7, GABRP, COMP, FNDC1, a fragment thereof, a complement thereof or a combination thereof.

In some embodiments, a method of diagnosing pancreatic neuroendocrine carcinoma or as a person suspected of having pancreatic cancer comprises determining the amount of protein in a subject of encoded by a sequence selected from: WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, a fragment thereof, a complement thereof or a combination thereof.

In some embodiments, the present invention provides methods of utilizing the promoter sequences of genes disclosed herein including: PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a sequence disclosed in Table 1 or Table 4, homologs thereof, combinations thereof, or fragments thereof, to express a transgene that results in the destruction or inhibits the growth, migration, or angiogenesis, of tumors and cells residing in tumors. By nonlimiting example, said promoter and transgene sequence may be expressed in exogenous cells such as perivascular cells, including mesenchymal stem cells, pericytes, RGS5 positive pericytes, or dispose stromal fraction cells that are introduced into the tumor or tumor site after the removal of the tumor, or into the blood circulation such that the exogenous cells activate the transgene subsequent to inhabiting the tumor site.

In some embodiments, the present invention provides methods of visualizing a tumor in a subject comprising targeting a cancer associated protein with a labeled molecule, wherein the cancer associated protein is selected from a protein described herein, and detecting the labeled molecule, wherein the labeled molecule visualizes the tumor in the subject. The protein may be selected from PPY, CHGB, ACE2, COL10A1, PRSS7, MEP1B, MMP12, MMP11, TM4SF2O, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP1, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a protein product of a sequence disclosed in Table 1, 2 and 6, homologs thereof, combinations thereof, or fragments thereof.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

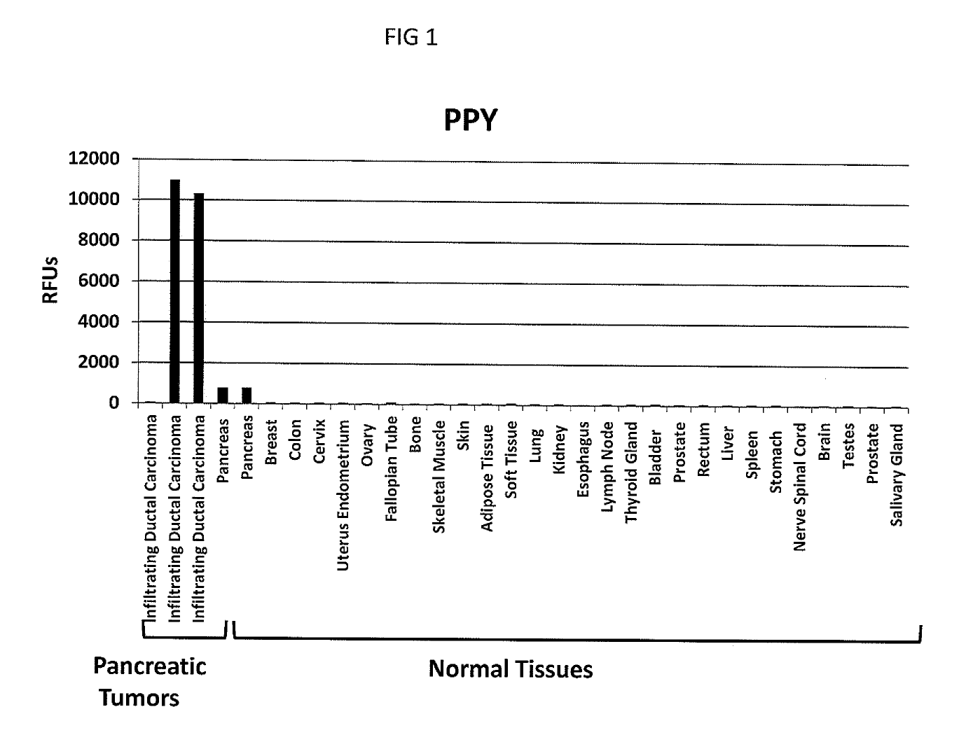

PPY: PPY (Accession number NM_{—}002722.3) encodes Homo sapiens pancreatic polypeptide. It is disclosed here that PPY is a novel marker for pancreas tumors. As shown in FIG. 1, PPY expression was assayed by Illumina microarray, a probe specific for PPY (probe sequence GGACTTATAATGCCACCTTCTGTCTCCTACGACTCCATGAGCAGCGCCAG (SEQ ID NO: 77); Illumina probe ID ILMN_{—}1751445) detected strong gene expression (>800 RFUs) in pancreas gland tumor adenocarcinoma, infiltrating ductal carcinoma. In contrast, expression of PPY in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<800 RFUs). The specificity of elevated PPY expression in malignant tumors of pancreas origin shown herein demonstrates that PPY is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target PPY can be identified using the methods described herein and therapeutics that target PPY include, but are not limited to, antibodies that modulate the activity of PPY. The manufacture and use of antibodies are described herein.

### Example 2

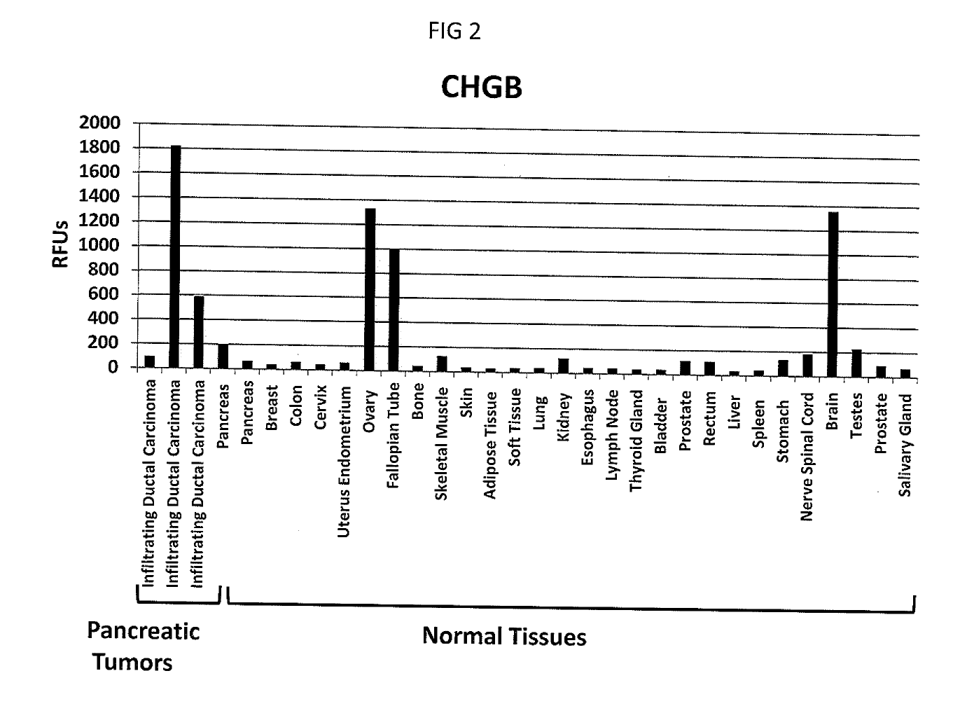

CHGB: CHGB (Accession number NM_{—}001819.1) encodes Homo sapiens chromogranin B (secretogranin 1). It is disclosed here that CHGB is a novel marker for pancreas tumors. As shown in FIG. 2, CHGB expression was assayed by Illumina microarray, a probe specific for CHGB (probe sequence TCAGCCAAAGGGGCTGACTGTCATTGGAGCGGTGGGCACTGTTAAGAAGC(SEQ ID NO: 78); Illumina probe ID ILMN_{—}1765966) detected strong gene expression (>1400 RFUs) in pancreas gland tumor adenocarcinoma, infiltrating ductal carcinoma. In contrast, expression of CHGB in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<1400 RFUs). The specificity of elevated CHGB expression in malignant tumors of pancreas origin shown herein demonstrates that CHGB is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target CHGB can be identified using the methods described herein and therapeutics that target CHGB include, but are not limited to, antibodies that modulate the activity of CHGB. The manufacture and use of antibodies are described herein.

### Example 3

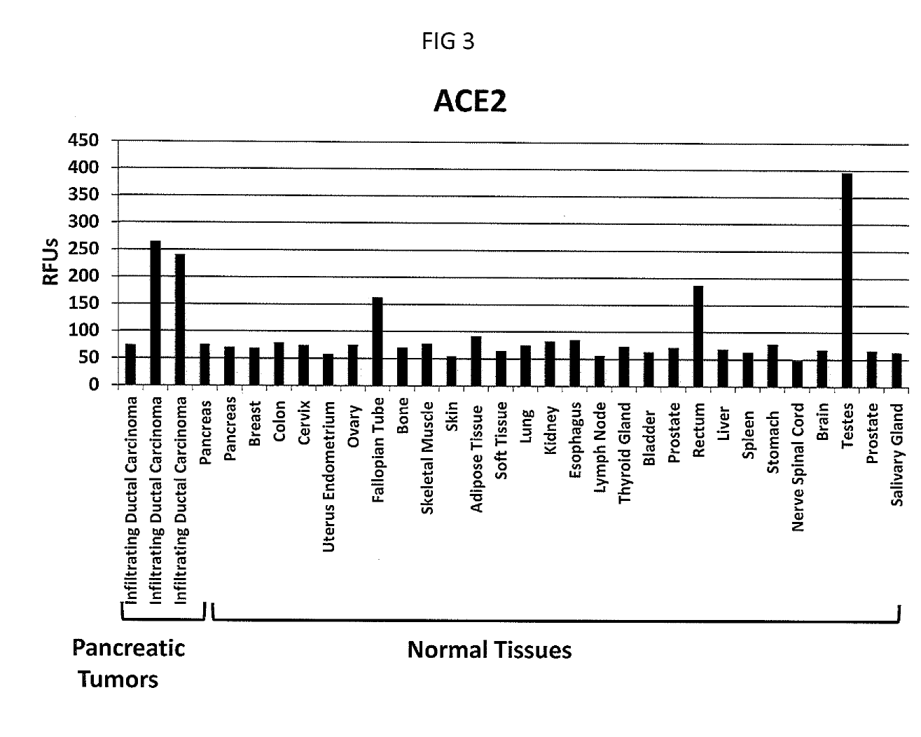

ACE2: ACE2 (Accession number NM_{—}021804.1) encodes Homo sapiens angiotensin 1 converting enzyme (peptidyl-dipeptidase A). It is disclosed here that ACE2 is a novel marker for pancreas tumors. As shown in FIG. 3, ACE2 expression was assayed by Illumina microarray, a probe specific for ACE2 (probe sequence ACATCTCCCTGACAACACAAAACTAGAGCCAGGGGCCTCCGTGAACTCCC (SEQ ID NO: 79); Illumina probe ID ILMN_{—}1667018) detected strong gene expression (>200 RFUs) in pancreas gland tumor adenocarcinoma, infiltrating ductal carcinoma. In contrast, expression of ACE2 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, pancreas, and salivary gland was generally low (<200 RFUs), with the exception of testis (397 RFUs). The specificity of elevated ACE2 expression in malignant tumors of pancreas origin shown herein demonstrates that ACE2 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target ACE2 can be identified using the methods described herein and therapeutics that target ACE2 include, but are not limited to, antibodies that modulate the activity of ACE2. The manufacture and use of antibodies are described herein.

### Example 4

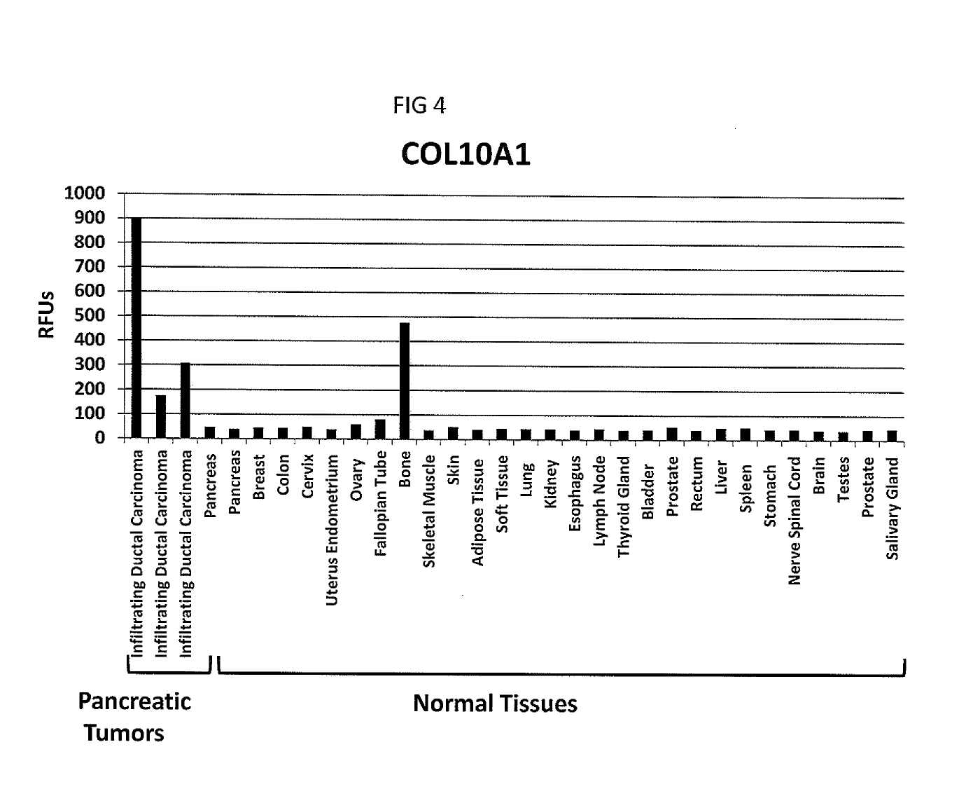

COL10A1: COL10A1 (Accession number NM_{—}000493.3) encodes Homo sapiens collagen, type X, alpha 1. It is disclosed here that COL10A1 is a novel marker for pancreas tumors. As shown in FIG. 4, COL10A1 expression was assayed by Illumina microarray, a probe specific for COL10A1 (probe sequence CCCCTAAAATATTTCTGATGGTGCACTACTCTGAGGCCTGTATGGCCCCT (SEQ ID NO: 80); Illumina probe ID ILMN_{—}1672776) detected strong gene expression (>100 RFUs) in pancreas gland tumor adenocarcinoma, infiltrating ductal carcinoma. In contrast, expression of COL10A1 in a wide variety of normal tissues including normal pancreas, kidney, breast. colon, rectum, cervix, endometrium, ovary, fallopian tube, skeletal muscle, skin, adipose tissue, lung, thyroid, esophagus, lymph node, pancreatic, prostate, liver, spleen, stomach, spinal cord, brain, testis, pancreas, and salivary gland was generally low (<100 RFUs), with the exception of bone (477 RFUs). The specificity of elevated COL10A1 expression in malignant tumors of pancreas origin shown herein demonstrates that COL10A1 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target COL10A1 can be identified using the methods described herein and therapeutics that target COL10A1 include, but are not limited to, antibodies that modulate the activity of COL10A1. The manufacture and use of antibodies are described herein.

### Example 5

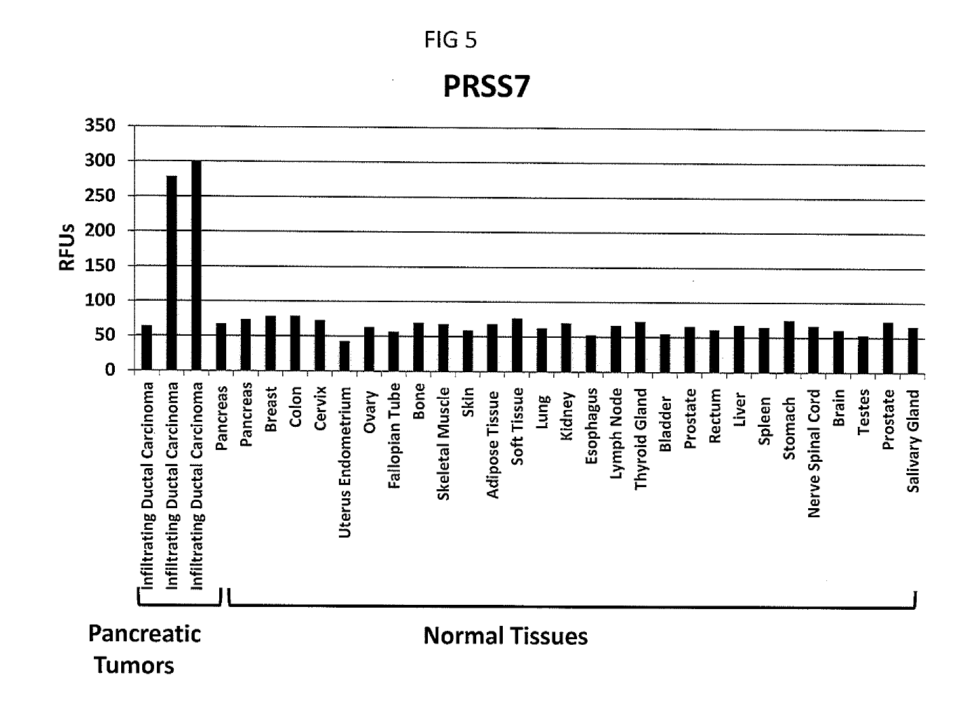

PRSS7: PRSS7 (Accession number NM_{—}002772.1) encodes Homo sapiens protease, serine, 7 (enterokinase). It is disclosed here that PRSS7 is a novel marker for pancreas tumors. As shown in FIG. 5, PRSS7 expression was assayed by Illumina microarray, a probe specific for PRSS7 (probe sequence GCCTAATCGCCCCGGAGTGTATGCCAGGGTCTCAAGGTTTACCGAATGGA (SEQ ID NO: 81); Illumina probe ID ILMN_{—}1695969) detected strong gene expression (>300 RFUs) in pancreas gland tumor adenocarcinoma, infiltrating ductal carcinoma. In contrast, expression of PRSS7 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<300 RFUs). The specificity of elevated PRSS7 expression in malignant tumors of pancreas origin shown herein demonstrates that PRSS7 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target PRSS7 can be identified using the methods described herein and therapeutics that target PRSS7 include, but are not limited to, antibodies that modulate the activity of PRSS7. The manufacture and use of antibodies are described herein.

### Example 6

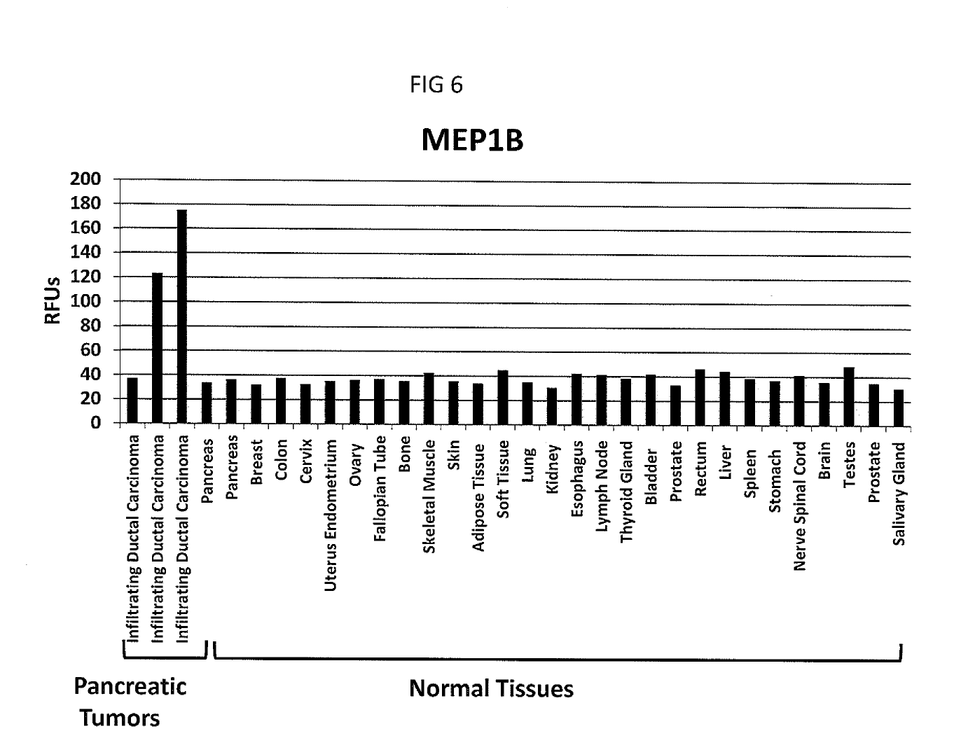

MEP1B: MEP1B (Accession number NM_{—}005925.2) encodes Homo sapiens meprin A, beta. It is disclosed here that MEP1B is a novel marker for pancreas tumors. As shown in FIG. 6, MEP1B expression was assayed by Illumina microarray, a probe specific for MEP1B (probe sequence GGTGGTACATGGGAGAAAGGTGTGAAAAGAGAGGCTCCACCCGAGACACC (SEQ ID NO: 82); Illumina probe ID ILMN_{—}1734694) detected strong gene expression (>100 RFUs) in pancreas gland tumor adenocarcinoma, infiltrating ductal carcinoma. In contrast, expression of MEP1B in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<100 RFUs). The specificity of elevated MEP1B expression in malignant tumors of pancreas origin shown herein demonstrates that MEP1B is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target MEP1B can be identified using the methods described herein and therapeutics that target MEP1B include, but are not limited to, antibodies that modulate the activity of MEP1B. The manufacture and use of antibodies are described herein.

### Example 7

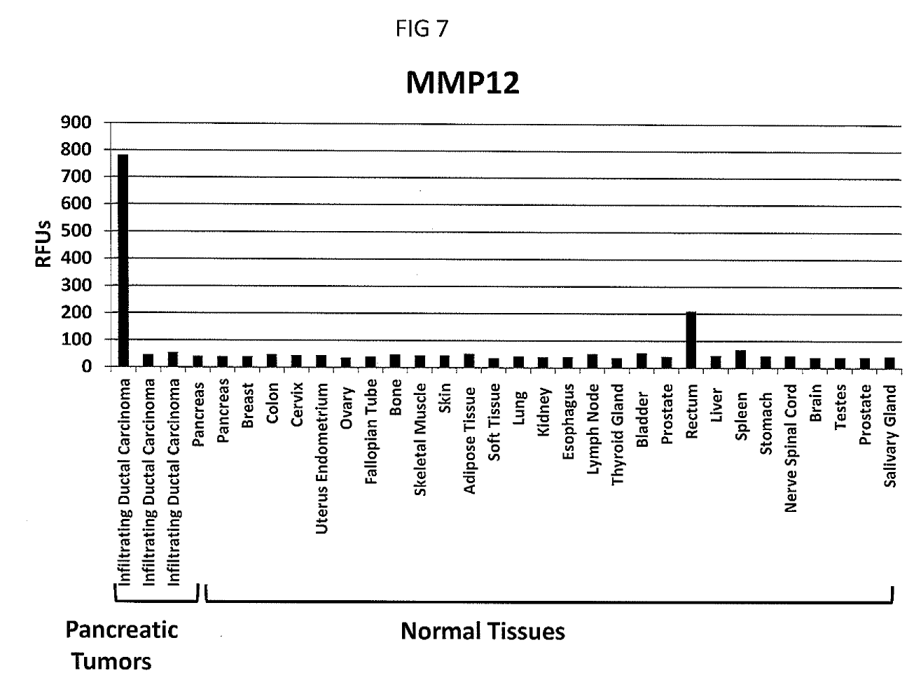

MMP12: MMP12 (Accession number NM_{—}002426.2) encodes Homo sapiens matrix metallopeptidase 12 (macrophage elastase). It is disclosed here that MMP12 is a novel marker for pancreas tumors. As shown in FIG. 7, MMP12 expression was assayed by Illumina microarray, a probe specific for MMP12 (probe sequence TCTATTTGAAGCATGCTCTGTAAGTTGCTTCCTAACATCCTTGGACTGAG (SEQ ID NO: 83); Illumina probe ID ILMN_{—}2073758) detected strong gene expression (>300 RFUs) in pancreas gland tumor adenocarcinoma, infiltrating ductal carcinoma. In contrast, expression of MMP12 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<300 RFUs). The specificity of elevated MMP12 expression in malignant tumors of pancreas origin shown herein demonstrates that MMP12 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target MMP12 can be identified using the methods described herein and therapeutics that target MMP12 include, but are not limited to, antibodies that modulate the activity of MMP12. The manufacture and use of antibodies are described herein.

### Example 8

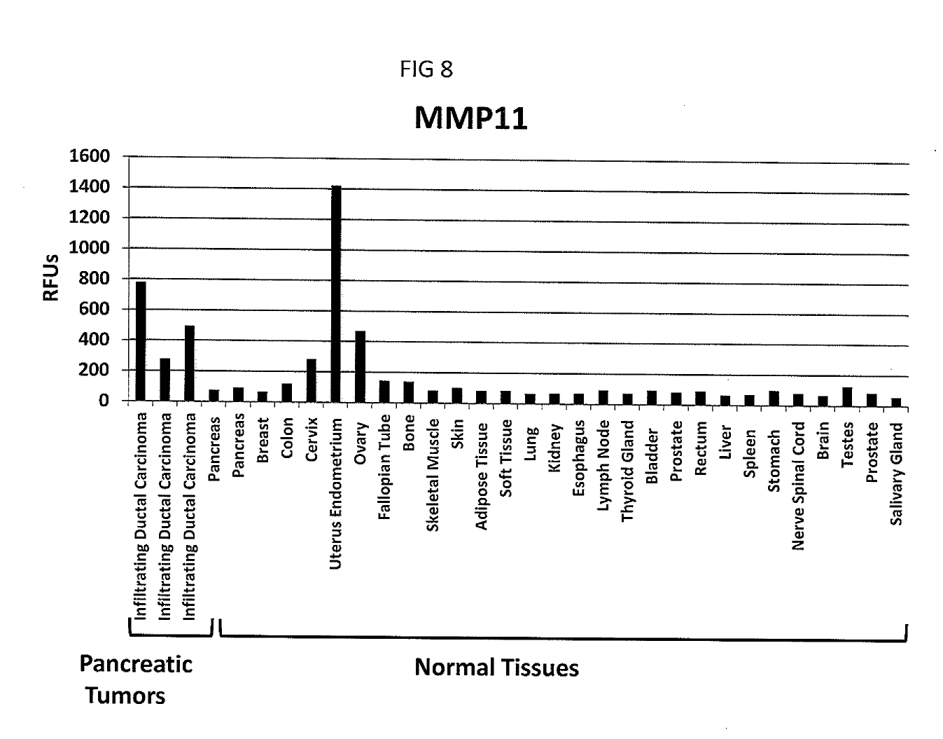

MMP11: MMP11 (Accession number NM_{—}005940.3) encodes Homo sapiens matrix metallopeptidase 11 (stromelysin 3). It is disclosed here that MMP11 is a novel marker for pancreas tumors. As shown in FIG. 8, MMP11 expression was assayed by Illumina microarray, a probe specific for MMP11 (probe sequence CAGGTCTTGGTAGGTGCCTGCATCTGTCTGCCTTCTGGCTGACAATCCTG (SEQ ID NO: 84); Illumina probe ID ILMN_{—}1655915) detected strong gene expression (>490 RFUs) in pancreas gland tumor adenocarcinoma, infiltrating ductal carcinoma. In contrast, expression of MMP11 in a wide variety of normal tissues including normal pancreas, kidney, breast. colon, rectum, cervix, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<490 RFUs), with the exception of endometrium (1425 RFUs). The specificity of elevated MMP11 expression in malignant tumors of pancreas origin shown herein demonstrates that MMP11 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target MMP11 can be identified using the methods described herein and therapeutics that target MMP11 include, but are not limited to, antibodies that modulate the activity of MMP11. The manufacture and use of antibodies are described herein.

### Example 9

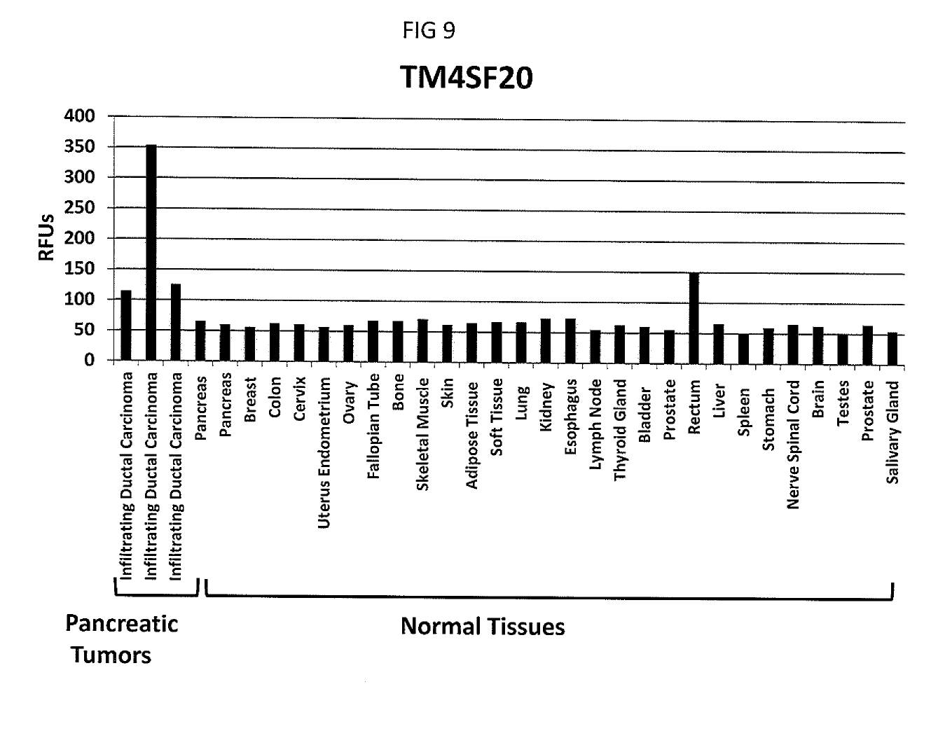

TM4SF20: TM4SF20 (Accession number NM_{—}024795.2) encodes Homo sapiens transmembrane 4 L six family member 20. It is disclosed here that TM4SF20 is a novel marker for pancreas tumors. As shown in FIG. 9, TM4SF20 expression was assayed by Illumina microarray, a probe specific for TM4SF20 (probe sequence GACTGCAACTCATCACATGAGGTCAGGTATGGAATTTTCCACTTGTGGTG (SEQ ID NO:857); Illumina probe ID ILMN_{—}1775830) detected strong gene expression (>100 RFUs) in pancreas gland tumor adenocarcinoma, infiltrating ductal carcinoma. In contrast, expression of TM4SF20 in a wide variety of normal tissues including normal pancreas, kidney, breast. colon, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<100 RFUs) with the exception of rectum (152 RFUs). The specificity of elevated TM4SF20 expression in malignant tumors of pancreas origin shown herein demonstrates that TM4SF20 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target TM4SF20 can be identified using the methods described herein and therapeutics that target TM4SF20 include, but are not limited to, antibodies that modulate the activity of TM4SF20. The manufacture and use of antibodies are described herein.

### Example 10

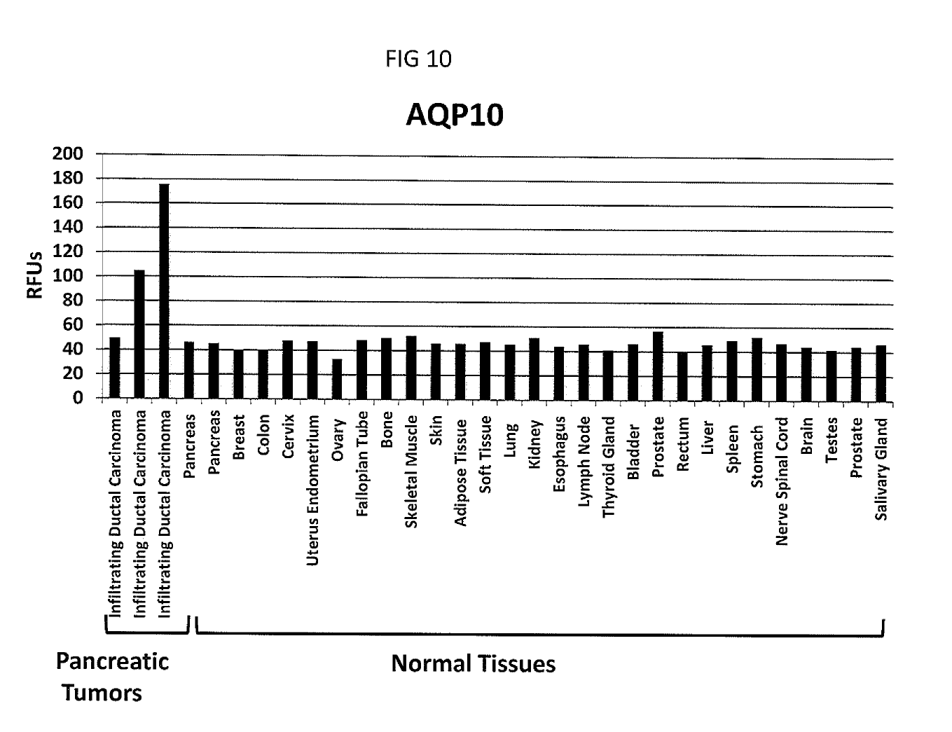

AQP10: AQP10 (Accession number NM_{—}080429.2) encodes Homo sapiens aquaporin 10. It is disclosed here that AQP10 is a novel marker for pancreas tumors. As shown in FIG. 10, AQP10 expression was assayed by IIlumina microarray, a probe specific for AQP10 (probe sequence CCCTGCAGAGGATGCTCGITTTGCAGAGAAGGCAGTGTTCCTCTATTCCC (SEQ ID NO: 86); Illumina probe ID ILMN_{—}2090004) detected strong gene expression (>100 RFUs) in pancreas gland tumor adenocarcinoma, infiltrating ductal carcinoma. In contrast, expression of AQP10 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<100 RFUs). The specificity of elevated AQP10 expression in malignant tumors of pancreas origin shown herein demonstrates that AQP10 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target AQP10 can be identified using the methods described herein and therapeutics that target AQP10 include, but are not limited to, antibodies that modulate the activity of AQP10. The manufacture and use of antibodies are described herein.

### Example 11

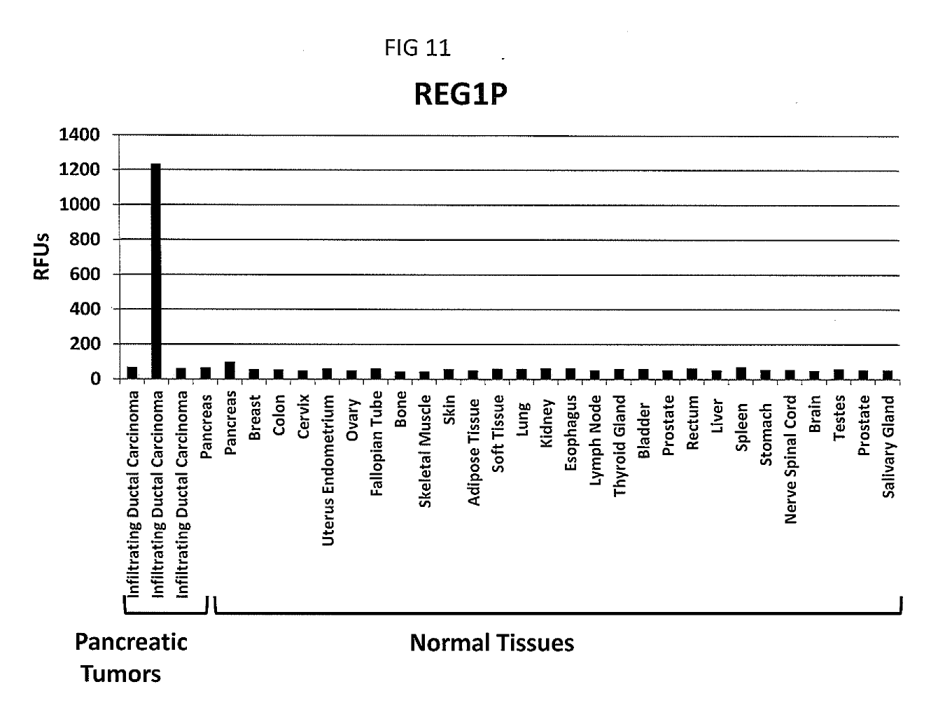

REG1P: REG1P (Accession number NR_{—}002714.1) encodes Homo sapiens regenerating islet-derived 1 pseudogene. It is disclosed here that REG1P is a novel marker for pancreas tumors. As shown in FIG. 11, REG1P expression was assayed by Illumina microarray, a probe specific for REG1P (probe sequence CTATTTATCTCTGAGGATCGACCGGGGACTGGGATAGAGGAAGGGTGAG (SEQ ID NO: 87) C; Illumina probe ID ILMN_{—}1785302) detected strong gene expression (>200 RFUs) in pancreas gland tumor adenocarcinoma, infiltrating ductal carcinoma. In contrast, expression of REG1P in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<200 RFUs). The specificity of elevated REG1P expression in malignant tumors of pancreas origin shown herein demonstrates that REG1P is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target REG1P can be identified using the methods described herein and therapeutics that target REG1P include, but are not limited to, antibodies that modulate the activity of REG1P. The manufacture and use of antibodies are described herein.

### Example 12

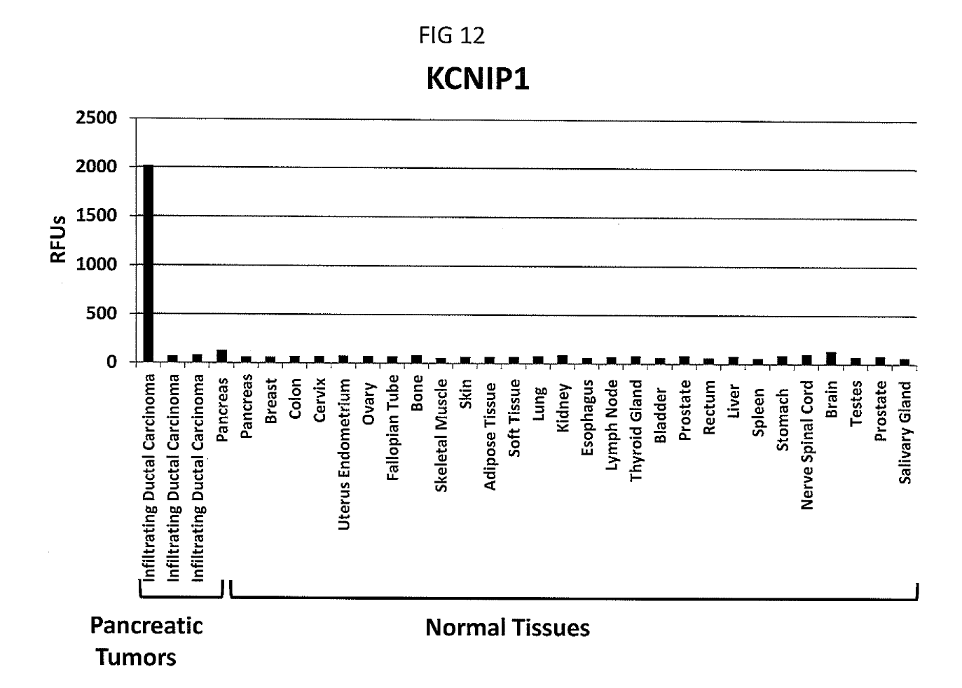

KCNIP1: KCNIP1 (Accession number NM_{—}014592.2) encodes Homo sapiens Kv channel interacting protein 1. It is disclosed here that KCNIP1 is a novel marker for pancreas tumors. As shown in FIG. 12, KCNIP1 expression was assayed by Illumina microarray, a probe specific for KCNIP1 (probe sequence GAGGTCTCTCCAGCTGTTTCAAAATGTCATGTAACTGGTGACACTCAGCC (SEQ ID NO: 88); Illumina probe ID ILMN_{—}2368856) detected strong gene expression (>200 RFUs) in pancreas gland tumor adenocarcinoma, infiltrating ductal carcinoma. In contrast, expression of KCNIP1 in a wide variety of normal tissues including normal pancreas, kidney, breast colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<200 RFUs). The specificity of elevated KCNIP1 expression in malignant tumors of pancreas origin shown herein demonstrates that KCNIP1 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target KCNIP1 can be identified using the methods described herein and therapeutics that target KCNIP1 include, but are not limited to, antibodies that modulate the activity of KCNIP1. The manufacture and use of antibodies are described herein.

### Example 13

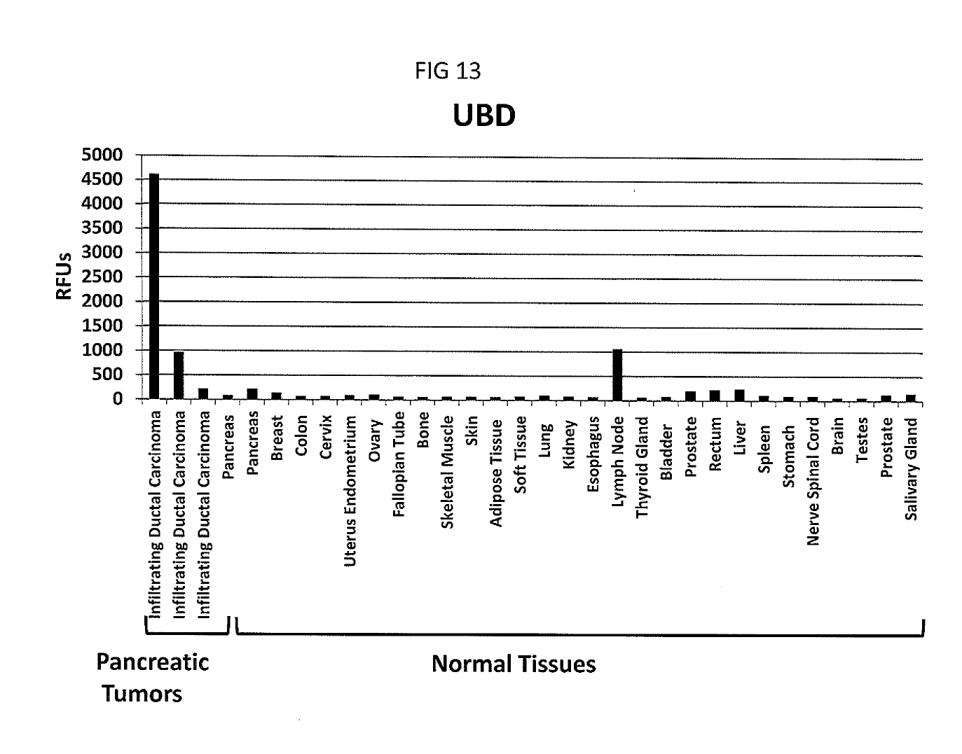

UBD: UBD (Accession number NM_{—}006398.2) encodes Homo sapiens ubiquitin D. It is disclosed here that UBD is a novel marker for pancreas tumors. As shown in FIG. 13, UBD expression was assayed by Illumina microarray, a probe specific for UBD (probe sequence CCTCCTCCAGGTGCGAAGGTCCAGCTCAGTGGCACAAGTGAAAGCAATGA (SEQ ID NO: 89); Illumina probe ID ILMN_{—}1678841) detected strong gene expression (>900 RFUs) in pancreas gland tumor adenocarcinoma, infiltrating ductal carcinoma. In contrast, expression of UBD in a wide variety of normal tissues including normal pancreas, kidney, breast. colon, rectum, cervix, endometrium, ovary, fallopian tube, bone, skeletal muscle, skin, adipose tissue, lung, thyroid, esophagus, pancreatic, prostate, liver, spleen, stomach, spinal cord, brain, testis, pancreas, and salivary gland was generally low (<900 RFUs), with the exception of lymph node (1076 RFUs). The specificity of elevated UBD expression in malignant tumors of pancreas origin shown herein demonstrates that UBD is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target UBD can be identified using the methods described herein and therapeutics that target UBD include, but are not limited to, antibodies that modulate the activity of UBD. The manufacture and use of antibodies are described herein.

### Example 14

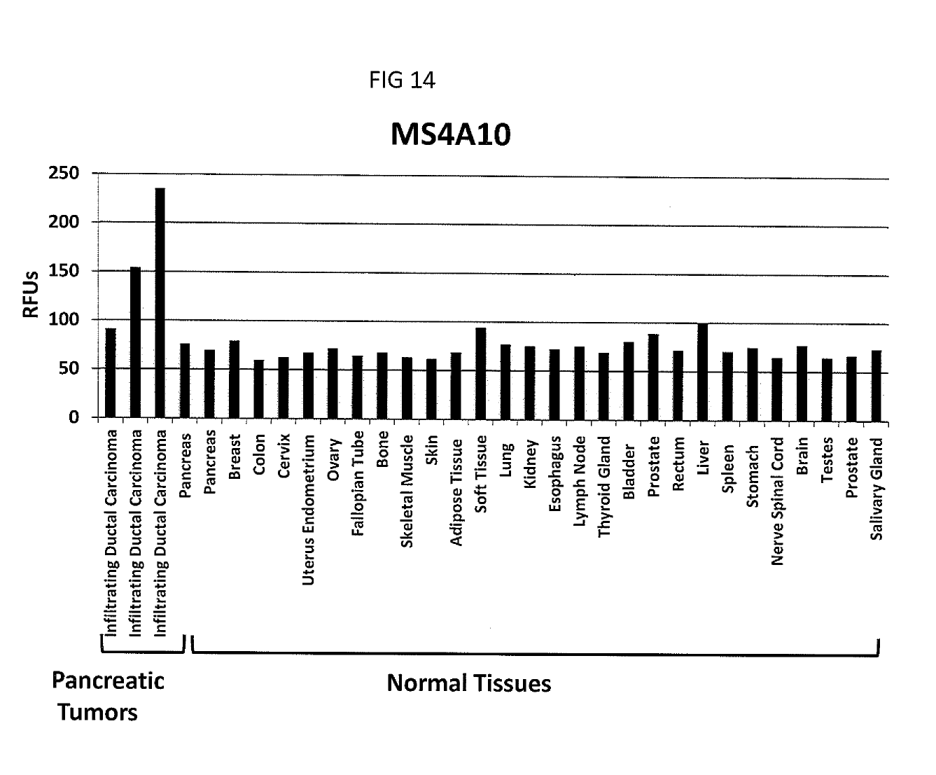

MS4A10: MS4A10 (Accession number NM_{—}206893.2) encodes Homo sapiens membrane-spanning 4-domains, subfamily A, member 10. It is disclosed here that MS4A10 is a novel marker for pancreas tumors. As shown in FIG. 14, MS4A10 expression was assayed by Illumina microarray, a probe specific for MS4A10 (probe sequence GGCTAGCTCTGCCAATCACTTACTGTGCGGGTTTGACTCAGTCCCTTCCC (SEQ ID NO: 90); Illumina probe ID ILMN_{—}1794921) detected strong gene expression (>120 RFUs) in pancreas gland tumor adenocarcinoma, infiltrating ductal carcinoma. In contrast, expression of MS4A10 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<120 RFUs). The specificity of elevated MS4A10 expression in malignant tumors of pancreas origin shown herein demonstrates that MS4A10 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target MS4A10 can be identified using the methods described herein and therapeutics that target MS4A10 include, but are not limited to, antibodies that modulate the activity of MS4A10. The manufacture and use of antibodies are described herein.

### Example 15

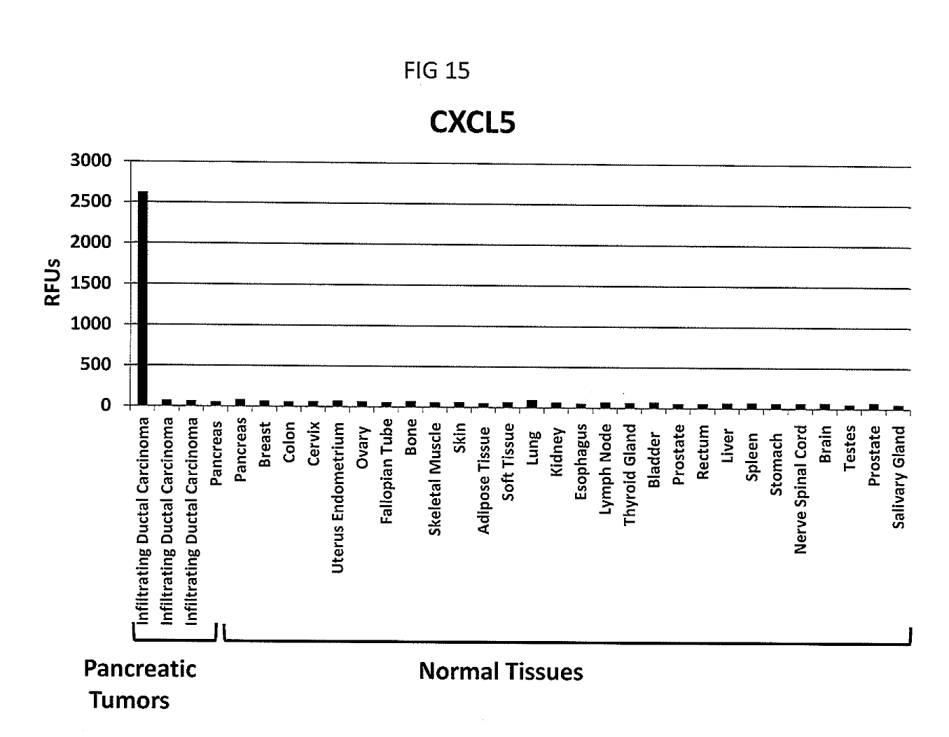

CXCL5: CXCL5 (Accession number NM_{—}002994.3) encodes Homo sapiens chemokine (C—X—C motif) ligand 5. It is disclosed here that CXCL5 is a novel marker for pancreas tumors. As shown in FIG. 15, CXCL5 expression was assayed by Illumina microarray, a probe specific for CXCL5. (probe sequence GCAAGTTCCCTCCCCACTCACAGCTTTGGCCCCTTTCACAGAGTAGAACC (SEQ ID NO: 91); Illumina probe ID ILMN_{—}1752562) detected strong gene expression (>100 RFUs) in pancreas gland tumor adenocarcinoma, infiltrating ductal carcinoma. In contrast, expression of CXCL5 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<100 RFUs). The specificity of elevated CXCL5 expression in malignant tumors of pancreas origin shown herein demonstrates that CXCL5 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target CXCL5 can be identified using the methods described herein and therapeutics that target CXCL5 include, but are not limited to, antibodies that modulate the activity of CXCL5. The manufacture and use of antibodies are described herein.

### Example 16

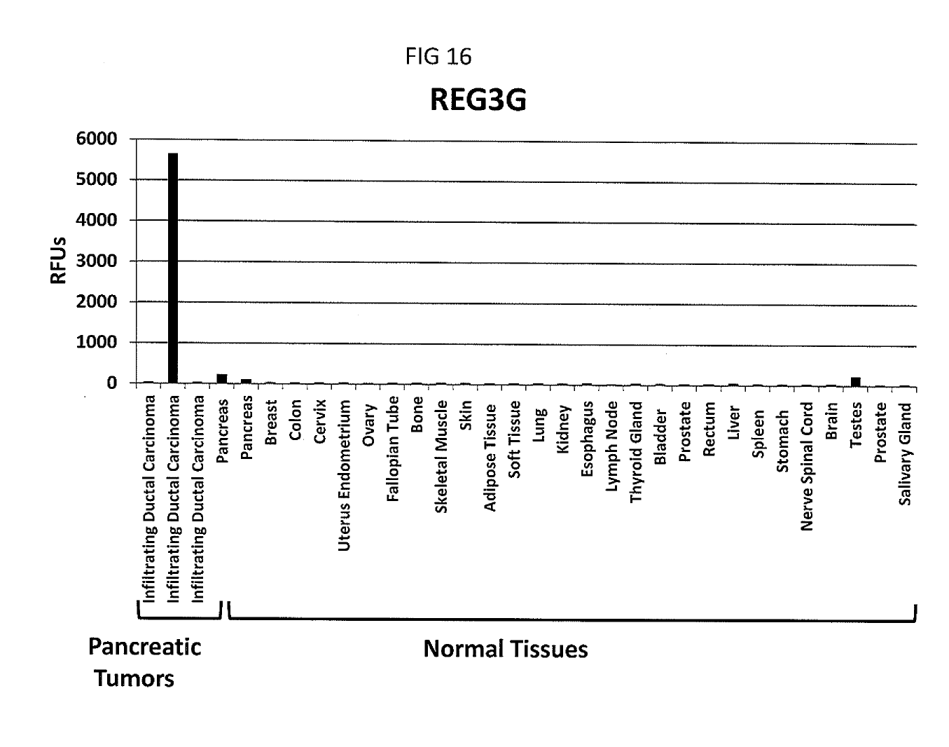

REG3G: REG3G (Accession number NM_{—}001008387.1) encodes Homo sapiens regenerating islet-derived 3 gamma. It is disclosed here that REG3G is a novel marker for pancreas tumors. As shown in FIG. 16, REG3G expression was assayed by Illumina microarray, a probe (probe specific for REG3G sequence CTGTGTGTCCTCCCGCTGACCACACTTCCTTTAGTGACCCGATTGCCTCC (SEQ ID NO: 92); Illumina probe ID ILMN_{—}1676795) detected strong gene expression (>250 RFUs) in pancreas gland tumor adenocarcinoma, infiltrating ductal carcinoma. In contrast, expression of REG3G in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<250 RFUs). The specificity of elevated REG3G expression in malignant tumors of pancreas origin shown herein demonstrates that REG3G is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target REG3G can be identified using the methods described herein and therapeutics that target REG3G include, but are not limited to, antibodies that modulate the activity of REG3G. The manufacture and use of antibodies are described herein.

### Example 17

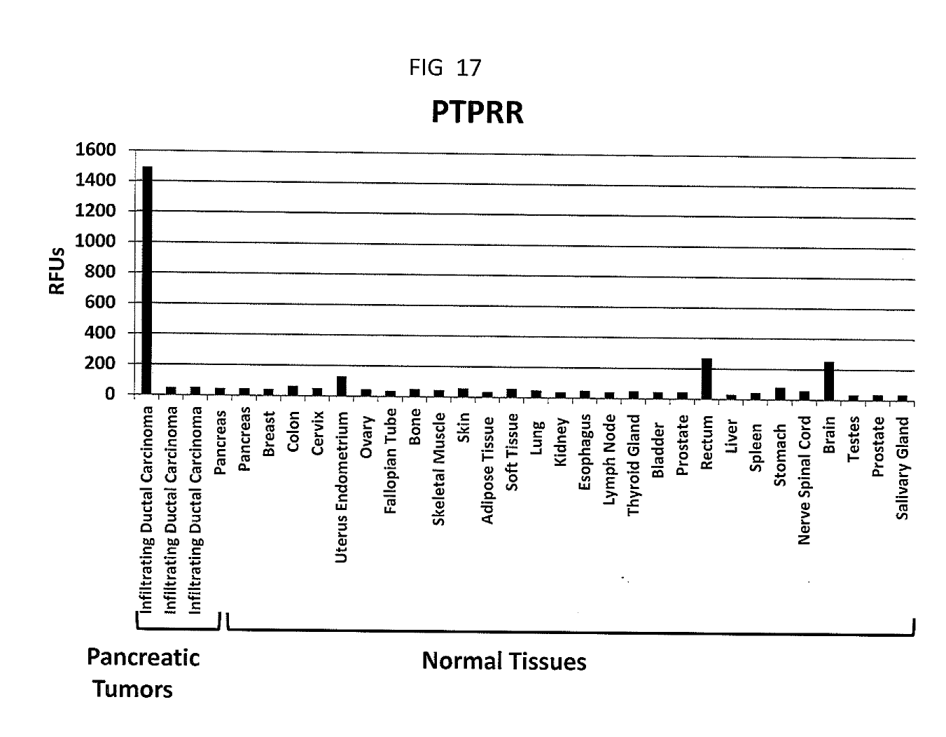

PTPRR: PTPRR (Accession number NM_{—}002849.2) encodes Homo sapiens protein tyrosine phosphatase, receptor type, R. It is disclosed here that PTPRR is a novel marker for pancreas tumors. As shown in FIG. 17, PTPRR expression was assayed by Illumina microarray, a probe specific for PTPRR (probe sequence GGTGGCTTCTGCTCCAGAACTCTATCCACTGTATTTCCACATCGTGAGTC (SEQ ID NO: 93); Illumina probe ID ILMN_{—}1679051) detected strong gene expression (>300 RFUs) in pancreas gland tumor adenocarcinoma, infiltrating ductal carcinoma. In contrast, expression of PTPRR in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<300 RFUs). The specificity of elevated PTPRR expression in malignant tumors of pancreas origin shown herein demonstrates that PTPRR is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target PTPRR can be identified using the methods described herein and therapeutics that target PTPRR include, but are not limited to, antibodies that modulate the activity of PTPRR. The manufacture and use of antibodies are described herein.

### Example 18

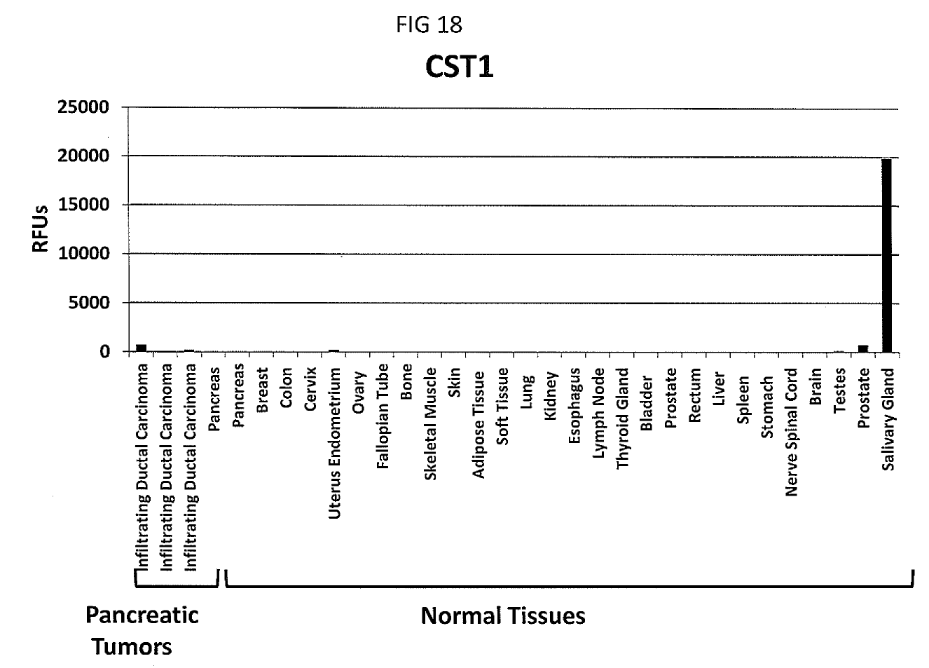

CST1: CST1 (Accession number NM_{—}001898.2) encodes Homo sapiens cystatin SN. It is disclosed here that CST1 is a novel marker for pancreas tumors. As shown in FIG. 18, CST1 expression was assayed by Illumina microarray, a probe specific for CST1 (probe sequence ATCCAGGTGTCAAGAATCCTAGGGATCTGTGCCAGGCCATTCGCACCAGC (SEQ ID NO: 94); Illumina probe ID ILMN_{—}1753449) detected strong gene expression (>500 RFUs) in pancreas gland tumor adenocarcinoma, infiltrating ductal carcinoma. In contrast, expression of CST1 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, liver, spleen, stomach, spinal cord, brain and testis was generally low (<500 RFUs), with the exception of prostate and salivary gland (783 and 19,929 RFUs respectively). The specificity of elevated CST1 expression in malignant tumors of pancreas origin shown herein demonstrates that CST1 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target CST1 can be identified using the methods described herein and therapeutics that target CST1 include, but are not limited to, antibodies that modulate the activity of CST1. The manufacture and use of antibodies are described herein.

### Example 19

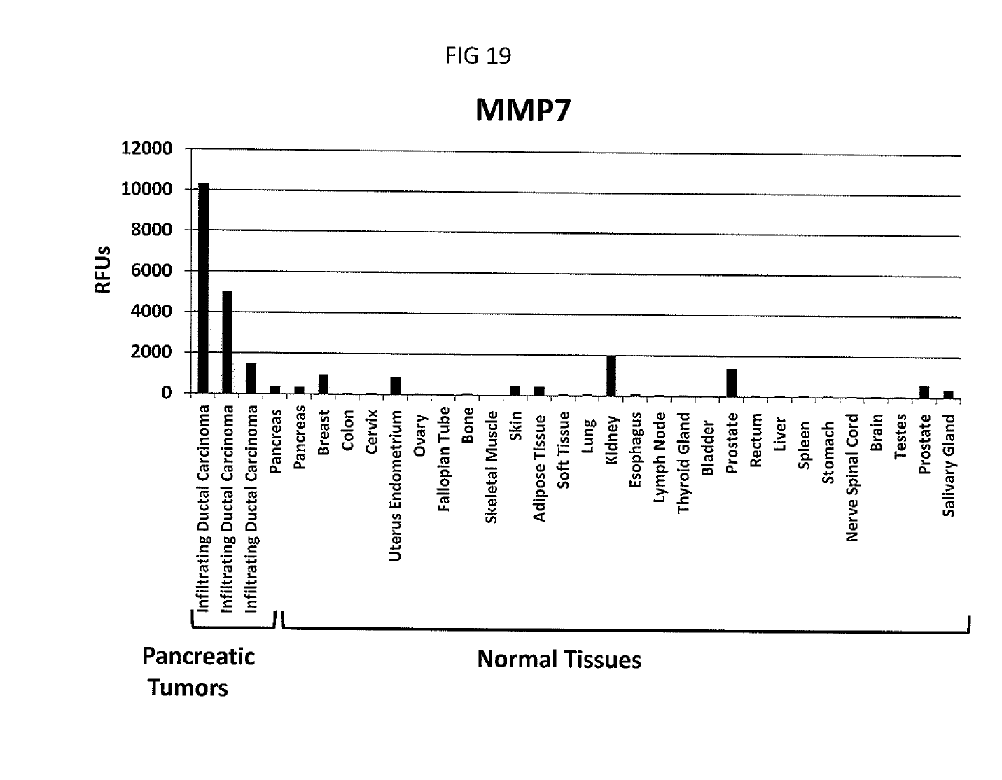

MMP7: MMP7 (Accession number NM_{—}002423.3) encodes Homo sapiens matrix metallopeptidase 7 (matrilysin, uterine. It is disclosed here that MMP7 is a novel marker for pancreas tumors. As shown in FIG. 19, MMP7 expression was assayed by Illumina microarray, a probe specific for MMP7 (probe sequence GCTCACTTCGATGAGGATGAACGCTGGACGGATGGTAGCAGTCTAGGGAT (SEQ ID NO: 95); Illumina probe ID ILMN_{—}1685403) detected strong gene expression (>1,400 RFUs) in pancreas gland tumor adenocarcinoma, infiltrating ductal carcinoma. In contrast, expression of MMP7 in a wide variety of normal tissues including normal pancreas, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<1,400 RFUs), with the exception of kidney (2,006 RFUs). The specificity of elevated MMP7 expression in malignant tumors of pancreas origin shown herein demonstrates that MMP7 is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target MMP7 can be identified using the methods described herein and therapeutics that target MMP7 include, but are not limited to, antibodies that modulate the activity of MMP7. The manufacture and use of antibodies are described herein.

### Example 20

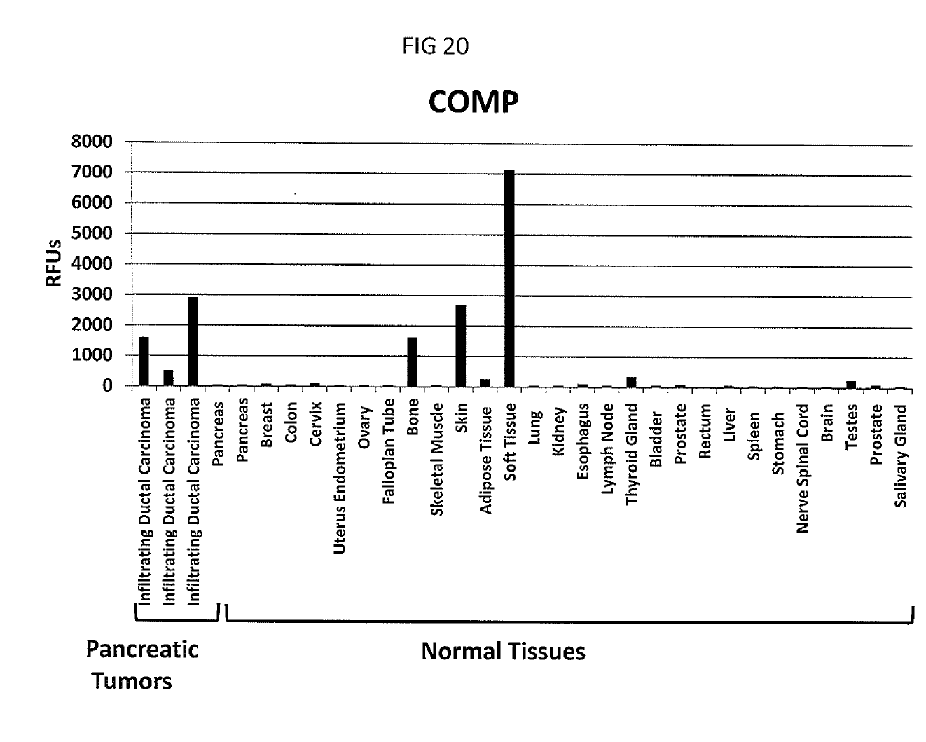

COMP: COMP (Accession number NM_{—}000095.2) encodes Homo sapiens cartilage oligomeric matrix protein. It is disclosed here that COMP is a novel marker for pancreas tumors. As shown in FIG. 20, COMP expression was assayed by Illumina microarray, a probe specific for COMP (probe sequence AGAGGACTATGAGACCCATCAGCTGCGGCAAGCCTAGGGACCAGGGTGAG (SEQ ID NO: 96); Illumina probe ID ILMN_{—}1677636) detected strong gene expression (>1,500 RFUs) in pancreas gland tumor adenocarcinoma, infiltrating ductal carcinoma. In contrast, expression of COMP in a wide variety of normal tissues including normal pancreas, kidney, breast. colon, rectum, cervix, endometrium, ovary, fallopian tube, skeletal muscle, adipose tissue, lung, thyroid, esophagus, lymph node, pancreatic, prostate, liver, spleen, stomach, spinal cord, brain, testis, pancreas, and salivary gland was generally low (<1,500 RFUs), with the exception of bone, skin and soft tissue (1,625, 2,690 and 7,142 RFUs respectively). The specificity of elevated COMP expression in malignant tumors of pancreas origin shown herein demonstrates that COMP is a marker for the diagnosis of pancreatic cancer (e.g. including but not limited to pancreatic adenocarcinomas and metastatic pancreatic tumors), and is a target for therapeutic intervention in pancreas cancer. The marker may be detected in urine as well as sera.

Therapeutics that target COMP can be identified using the methods described herein and therapeutics that target COMP include, but are not limited to, antibodies that modulate the activity of COMP. The manufacture and use of antibodies are described herein.

### Example 21

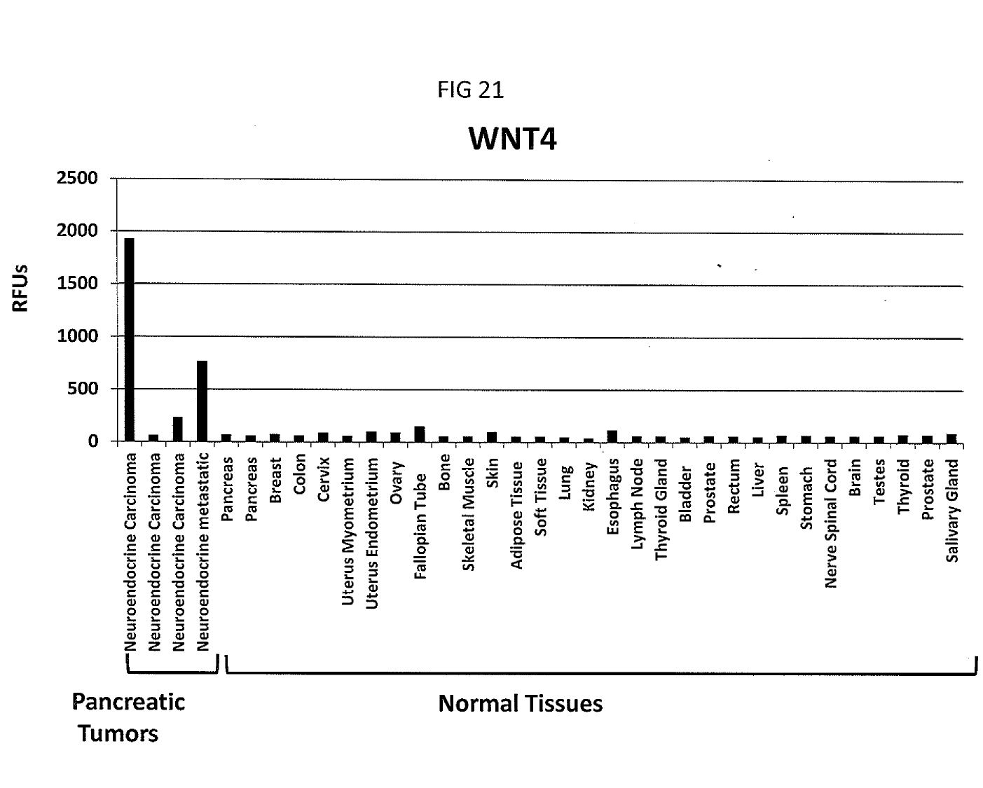

WNT4: WNT4 (Accession number NM_{—}030761.3) encodes Homo sapiens wingless-type MMTV integration site family, member 4. It is disclosed here that WNT4 is a novel marker for pancreas tumors. As shown in FIG. 21, WNT4 expression was assayed by Illumina microarray, a probe specific for WNT4 (probe sequence CCTGCGCCGGCAACCACCTAGTGGCCCAGGGAAGGCCGATAATTTAAACA (SEQ ID NO: 97); Illumina probe ID ILMN_{—}1666392) detected strong gene expression (>200 RFUs) in pancreas neuroendocrine tumors. In contrast, expression of WNT4 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<200 RFUs). The specificity of elevated WNT4 expression in malignant tumors of pancreas origin shown herein demonstrates that WNT4 is a marker for the diagnosis of pancreatic cancer (e.g. including but not limited to pancreatic neuroendocrine tumors and metastatic pancreatic tumors), and is a target for therapeutic intervention in pancreas cancer. The marker may be detected in urine as well as sera.

Therapeutics that target WNT4 can be identified using the methods described herein and therapeutics that target WNT4 include, but are not limited to, antibodies that modulate the activity of WNT4. The manufacture and use of antibodies are described herein.

### Example 22

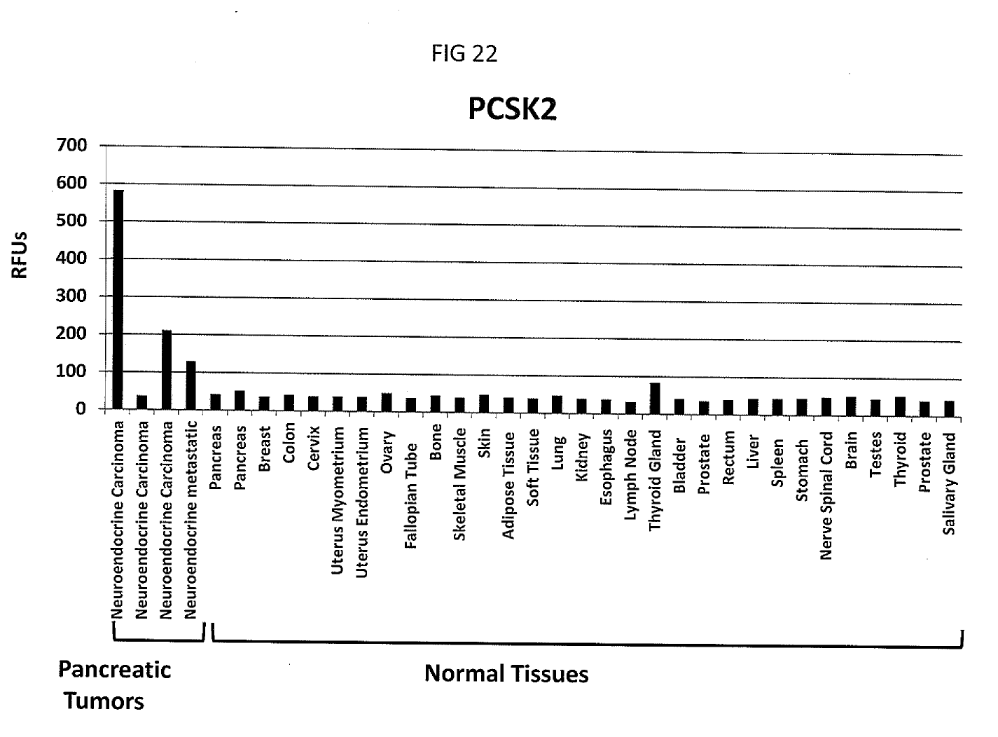

PCSK2; PCSK2 (Accession number NM_{—}002594.2) encodes Homo sapiens preprotein convertase subtilisin/kexin type 2. It is disclosed here that PCSK2 is a novel marker for pancreas tumors. As shown in FIG. 22, PCSK2 expression was assayed by IIlumina microarray, a probe specific for PCSK2 (probe sequence GGCCAGTGGAAATTCAGGTGAAAATGTTCATCAATTCCCATTGCATCACC (SEQ ID NO: 98); Illumina probe ID ILMN_{—}1695003) detected strong gene expression (>100 RFUs) in pancreas neuroendocrine tumors. In contrast, expression of PCSK2 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<100 RFUs). The specificity of elevated PCSK2 expression in malignant tumors of pancreas origin shown herein demonstrates that PCSK2 is a marker for the diagnosis of pancreatic cancer (e.g. including but not limited to pancreatic neuroendocrine tumors and metastatic pancreatic tumors), and is a target for therapeutic intervention in pancreas cancer. The marker may be detected in urine as well as sera.

Therapeutics that target PCSK2 can be identified using the methods described herein and therapeutics that target PCSK2 include, but are not limited to, antibodies that modulate the activity of PCSK2. The manufacture and use of antibodies are described herein.

### Example 23

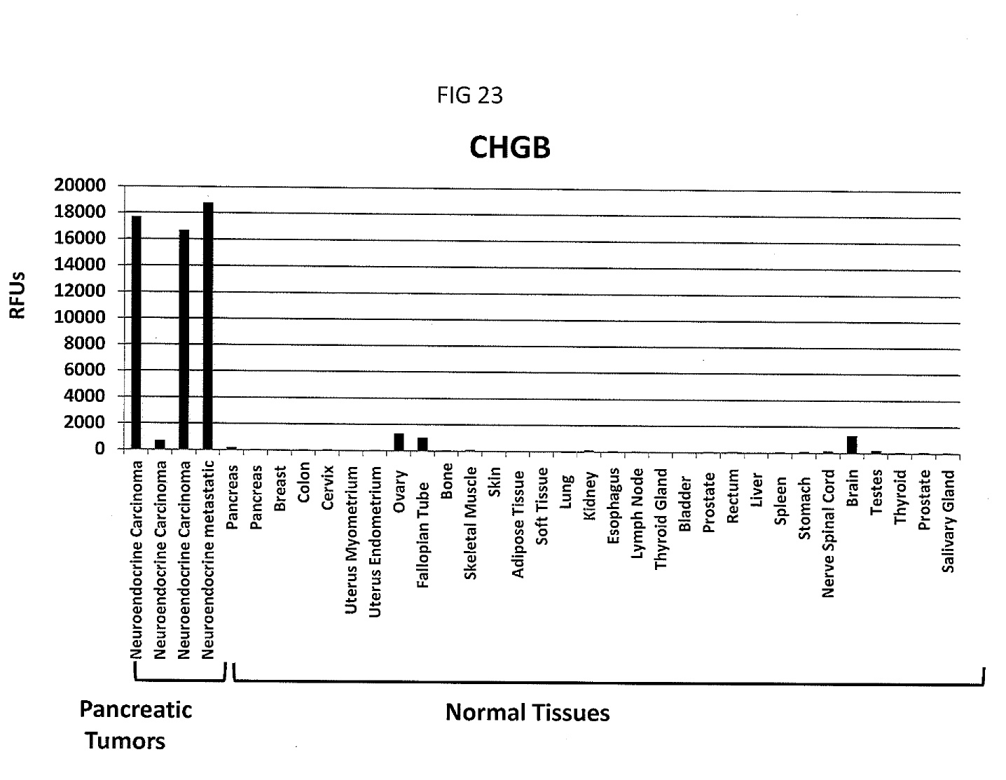

CHGB: CHGB (Accession number NM_{—}001819.1) encodes Homo sapiens chromogranin 13 (secretogranin 1). It is disclosed here that CHGB is a novel marker for pancreas tumors. As shown in FIG. 23, CHGB expression was assayed by Illumina microarray, a probe specific for CHGB (probe sequence TCAGCCAAAGGGGCTGACTGTCATTGGAGCGGTGGGCACTGTTAAGAAGC(SEQ ID NO: 99); Illumina probe ID ILMN_{—}1765966) detected strong gene expression (>1500 RFUs) in pancreas neuroendocrine tumors. In contrast, expression of CHGB in a wide variety of normal tissues including normal pancreas, kidney, breast, colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<1500 RFUs). The specificity of elevated CHGB expression in malignant tumors of pancreas origin shown herein demonstrates that CHGB is a marker for the diagnosis of pancreatic cancer (e.g. including but not limited to pancreatic neuroendocrine tumors and metastatic pancreatic tumors), and is a target for therapeutic intervention in pancreas cancer. The marker may be detected in urine as well as sera.

Therapeutics that target CHGB can be identified using the methods described herein and therapeutics that target CHGB include, but are not limited to, antibodies that modulate the activity of CHGB. The manufacture and use of antibodies are described herein.

### Example 24

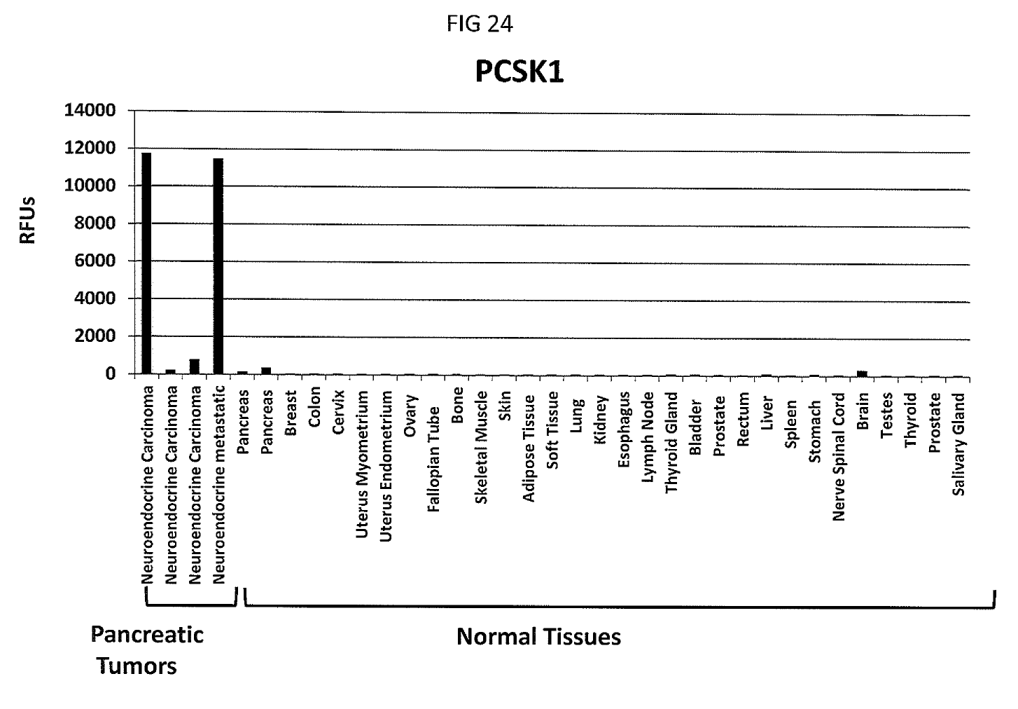

PCSK1: PCSK1 (Accession number NM_{—}000439.3) encodes Homo sapiens proprotein convertase subtilisin/kexin type 1. It is disclosed here that PCSK1 is a novel marker for pancreas tumors. As shown in FIG. 24, PCSK1 expression was assayed by Illumina microarray, a probe specific for PCSK1 (probe sequence GTAGAGGGTGTTTGCAGAGCAATGCCCGTAATGCTTAGAGAATGTTCTCC (SEQ ID NO: 100); Illumina probe ID ILMN_{—}1710859) detected strong gene expression (>400 RFUs) in pancreas neuroendocrine tumors. In contrast, expression of PCSK1 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<400 RFUs). The specificity of elevated PCSK1 expression in malignant tumors of pancreas origin shown herein demonstrates that PCSK1 is a marker for the diagnosis of pancreatic cancer (e.g. including but not limited to pancreatic neuroendocrine tumors and metastatic pancreatic tumors), and is a target for therapeutic intervention in pancreas cancer. The marker may be detected in urine as well as sera.

Therapeutics that target PCSK1 can be identified using the methods described herein and therapeutics that target PCSK1 include, but are not limited to, antibodies that modulate the activity of PCSK1. The manufacture and use of antibodies are described herein.

### Example 25

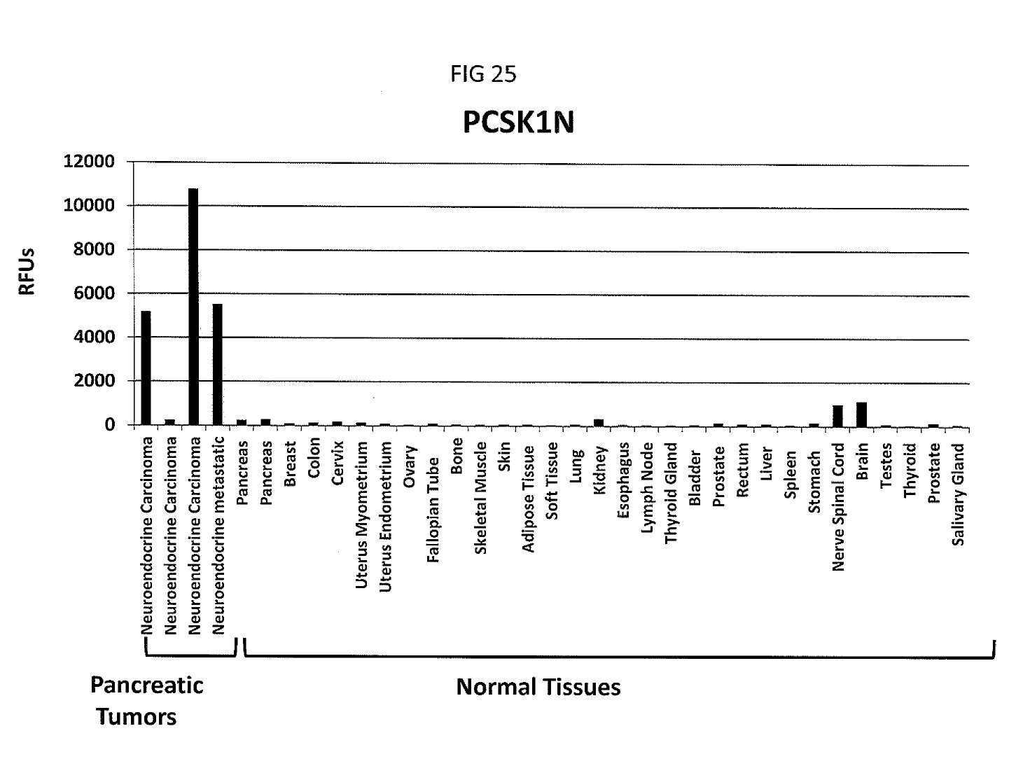

PCSK1N: PCSK1N (Accession number NM_{—}013271.2) encodes Homo sapiens proprotein convertase subtilisin/kexin type 1 inhibitor. It is disclosed here that PCSK1N is a novel marker for pancreas tumors. As shown in FIG. 25, PCSK1N expression was assayed by Illumina microarray, a probe specific for PCSK1N (probe sequence AGCTGTTGAGGTACTTGCTGGGACGGATTCTTGCGGGAAGCGCGGACTCC(SEQ ID NO: 101); Illumina probe ID ILMN_{—}1755582) detected strong gene expression (>1200 RFUs) in pancreas neuroendocrine tumors. In contrast, expression of PCSK1N in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<1200 RFUs). The specificity of elevated PCSK1N expression in malignant tumors of pancreas origin shown herein demonstrates that PCSK1N is a marker for the diagnosis of pancreatic cancer (e.g. including but not limited to pancreatic neuroendocrine tumors and metastatic pancreatic tumors), and is a target for therapeutic intervention in pancreas cancer. The marker may be detected in urine as well as sera.

Therapeutics that target PCSK1N can be identified using the methods described herein and therapeutics that target PCSK1N include, but are not limited to, antibodies that modulate the activity of PCSK1N. The manufacture and use of antibodies are described herein.

### Example 26

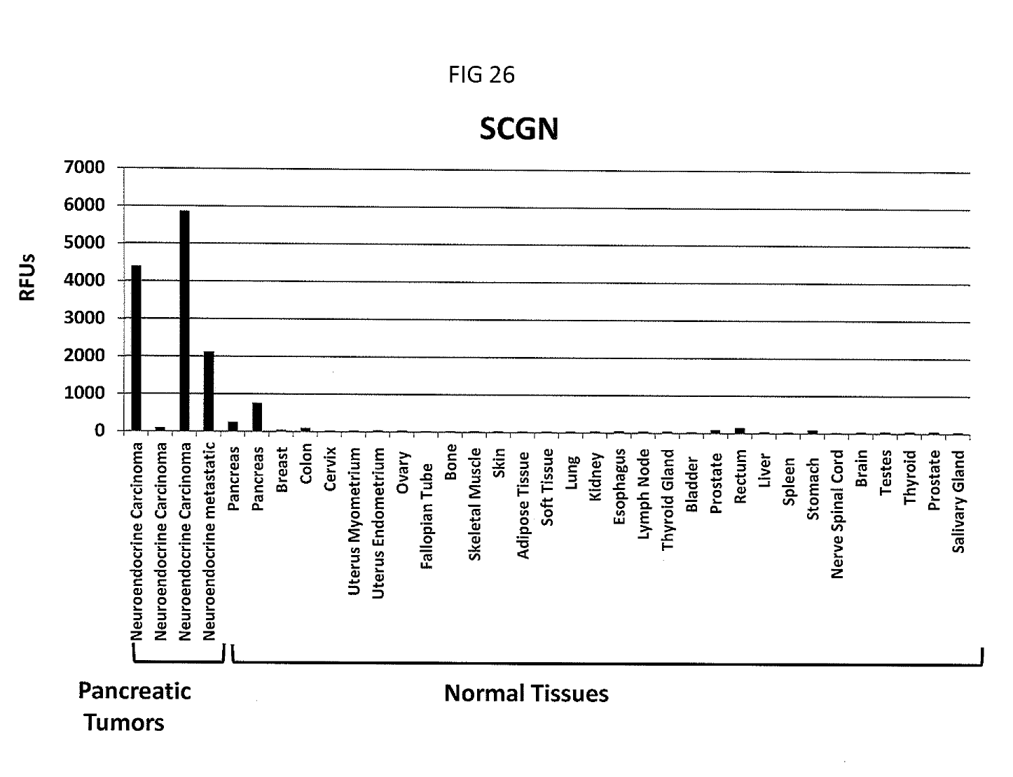

SCGN: SCGN (Accession number NM_{—}006998.3) encodes Homo sapiens secretagogin, EF-hand calcium binding protein. It is disclosed here that SCGN is a novel marker for pancreas tumors. As shown in FIG. 26, SCGN expression was assayed by Illumina microarray, a probe specific for SCGN (probe sequence CTCCCAAAGACTCAGCTCCCCTGTTAGATGGCTCTGCCTGTCCTTCCCCA (SEQ ID NO: 102); Illumina probe ID ILMN_{—}1789648) detected strong gene expression (>1000 RFUs) in pancreas neuroendocrine tumors. In contrast, expression of SCGN in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<1000 RFUs). The specificity of elevated SCGN expression in malignant tumors of pancreas origin shown herein demonstrates that SCGN is a marker for the diagnosis of pancreatic cancer (e.g. including but not limited to pancreatic neuroendocrine tumors and metastatic pancreatic tumors), and is a target for therapeutic intervention in pancreas cancer. The marker may be detected in urine as well as sera.

Therapeutics that target SCGN can be identified using the methods described herein and therapeutics that target SCGN include, but are not limited to, antibodies that modulate the activity of SCGN. The manufacture and use of antibodies are described herein.

### Example 27

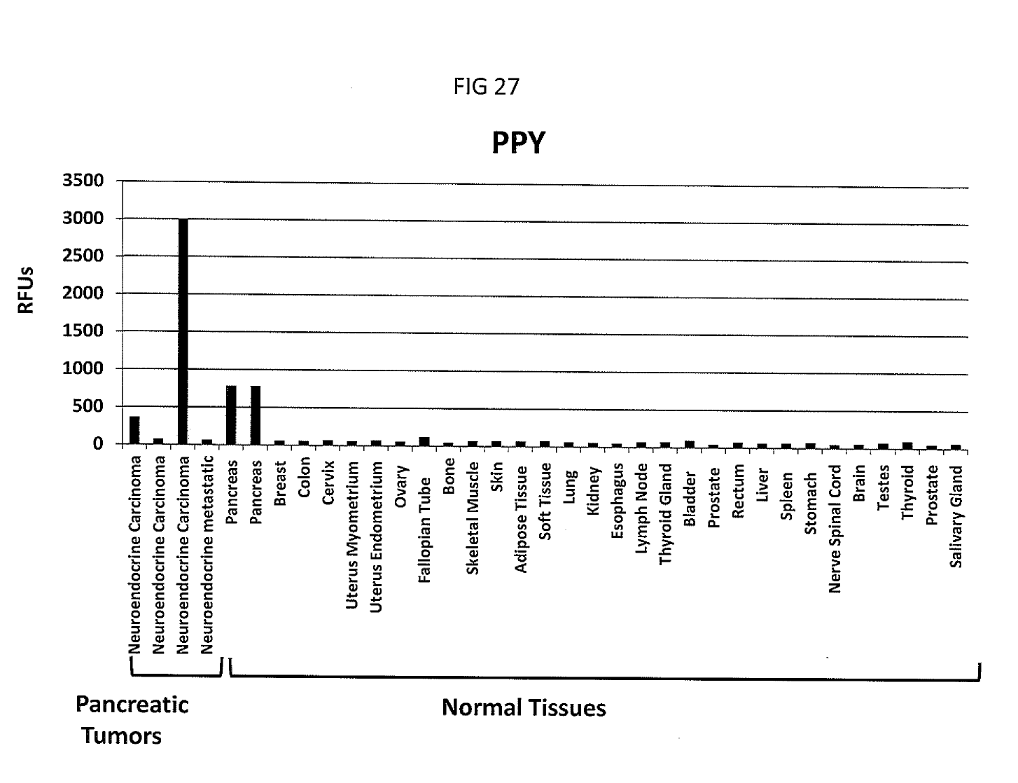

PPY: PPY (Accession number NM_{—}002722.3) encodes Homo sapiens pancreatic polypeptide. It is disclosed here that PPY is a novel marker for pancreas tumors. As shown in FIG. 27, PPY expression was assayed by Illumina microarray, a probe specific for PPY (probe sequence GGACTTATAATGCCACCTTCTGTCTCCTACGACTCCATGAGCAGCGCCAG (SEQ ID NO: 103); Illumina probe ID ILMN_{—}1751445) detected strong gene expression (>1000 RFUs) in pancreas neuroendocrine tumors. In contrast, expression of PPY in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<1000 RFUs). The specificity of elevated PPY expression in malignant tumors of pancreas origin shown herein demonstrates that PPY is a marker for the diagnosis of pancreatic cancer (e.g. including but not limited to pancreatic neuroendocrine tumors and metastatic pancreatic tumors), and is a target for therapeutic intervention in pancreas cancer. The marker may be detected in urine as well as sera.

Therapeutics that target PPY can be identified using the methods described herein and therapeutics that target PPY include, but are not limited to, antibodies that modulate the activity of PPY. The manufacture and use of antibodies are described herein.

### Example 28

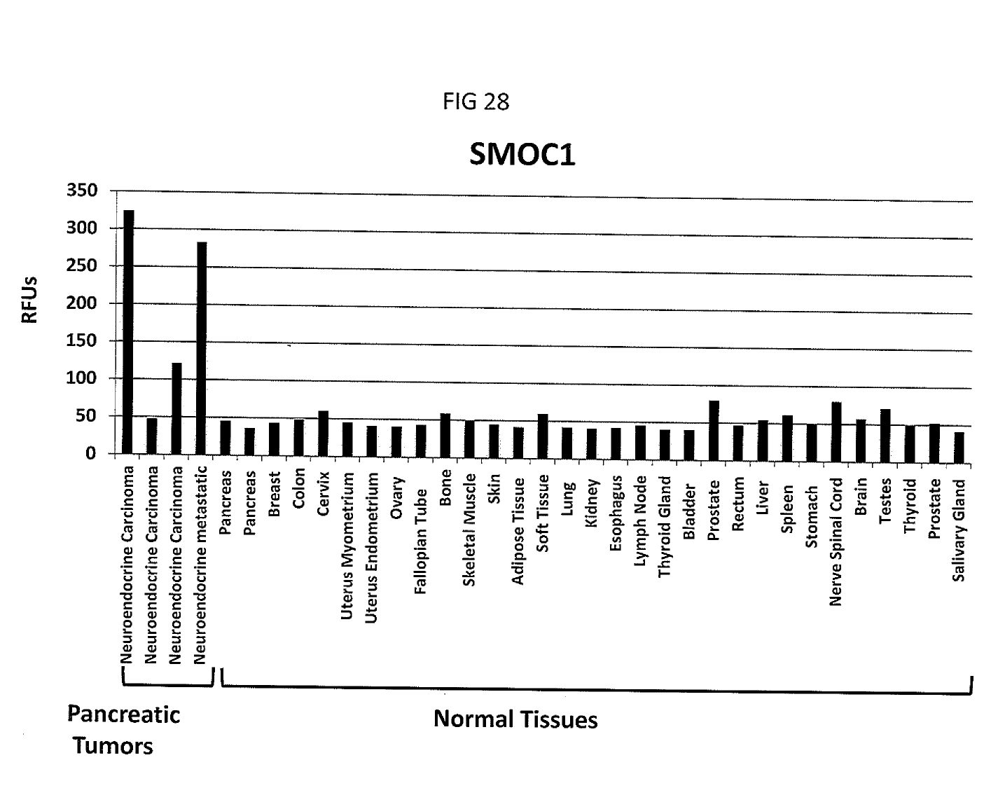

SMOC1: SMOC1 (Accession number NM_{—}001034852.1) encodes Homo sapiens SPARC related modular calcium binding I. It is disclosed here that SMOC1 is a novel marker for pancreas tumors. As shown in FIG. 28, SMOC1 expression was assayed by Illumina microarray, a probe specific for SMOC1 (probe sequence CAACAGGGAAACTCTTCACCTCCCTGCAAACCTACCAGTGAGGCTCCCAG (SEQ ID NO: 104); Illumina probe ID ILMN_{—}1791202) detected strong gene expression (>100 RFUs) in pancreas neuroendocrine tumors. In contrast, expression of SMOC1 in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<100 RFUs). The specificity of elevated SMOC1 expression in malignant tumors of pancreas origin shown herein demonstrates that SMOC1 is a marker for the diagnosis of pancreatic cancer (e.g. including but not limited to pancreatic neuroendocrine tumors and metastatic pancreatic tumors), and is a target for therapeutic intervention in pancreas cancer. The marker may be detected in urine as well as sera.

Therapeutics that target SMOC1 can be identified using the methods described herein and therapeutics that target SMOC1 include, but are not limited to, antibodies that modulate the activity of SMOC1. The manufacture and use of antibodies are described herein.

### Example 29

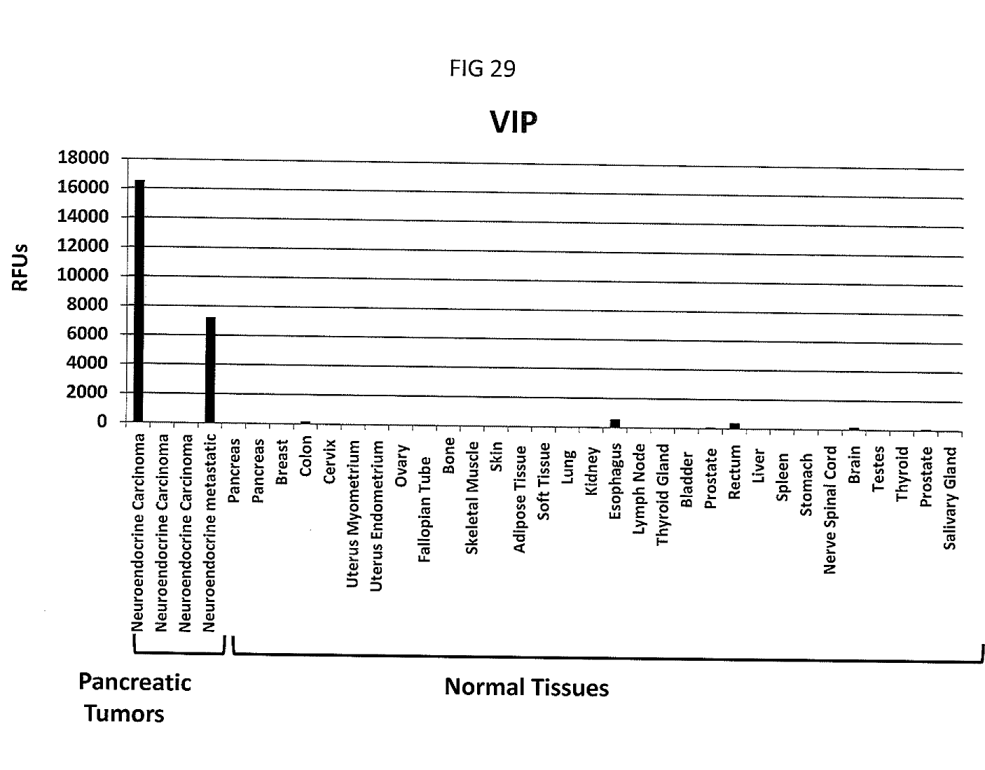

VIP: VIP (Accession number NM_{—}003381.2) encodes Homo sapiens vasoactive intestinal peptide. It is disclosed here that VIP is a novel marker for pancreas tumors. As shown in FIG. 29, VIP expression was assayed by Illumina microarray, a probe specific for VIP (probe sequence CTTCGGCATGGCCTCTTTACAGGGCACCTTCTGCTCTCAGGTTGGGTGAC (SEQ ID NO: 105); Illumina probe ID ILMN_{—}2357542) detected strong gene expression (>600 RFUs) in pancreas neuroendocrine tumors. In contrast, expression of VIP in a wide variety of normal tissues including normal pancreas, kidney, breast. colon, rectum, cervix, endometrium, ovary, fallopian tube, bone, skeletal muscle, skin, adipose tissue, lung, thyroid, esophagus, lymph node, pancreatic, prostate, liver, spleen, stomach, spinal cord, brain, testis, pancreas, and salivary gland was generally low (<600 RFUs). The specificity of elevated VIP expression in malignant tumors of pancreas origin shown herein demonstrates that VIP is a marker for the diagnosis of pancreatic cancer (e.g. including but not limited to pancreatic neuroendocrine tumors and metastatic pancreatic tumors), and is a target for therapeutic intervention in pancreas cancer. The marker may be detected in urine as well as sera.

Therapeutics that target VIP can be identified using the methods described herein and therapeutics that target VIP include, but are not limited to, antibodies that modulate the activity of VIP. The manufacture and use of antibodies are described herein.

### Example 30

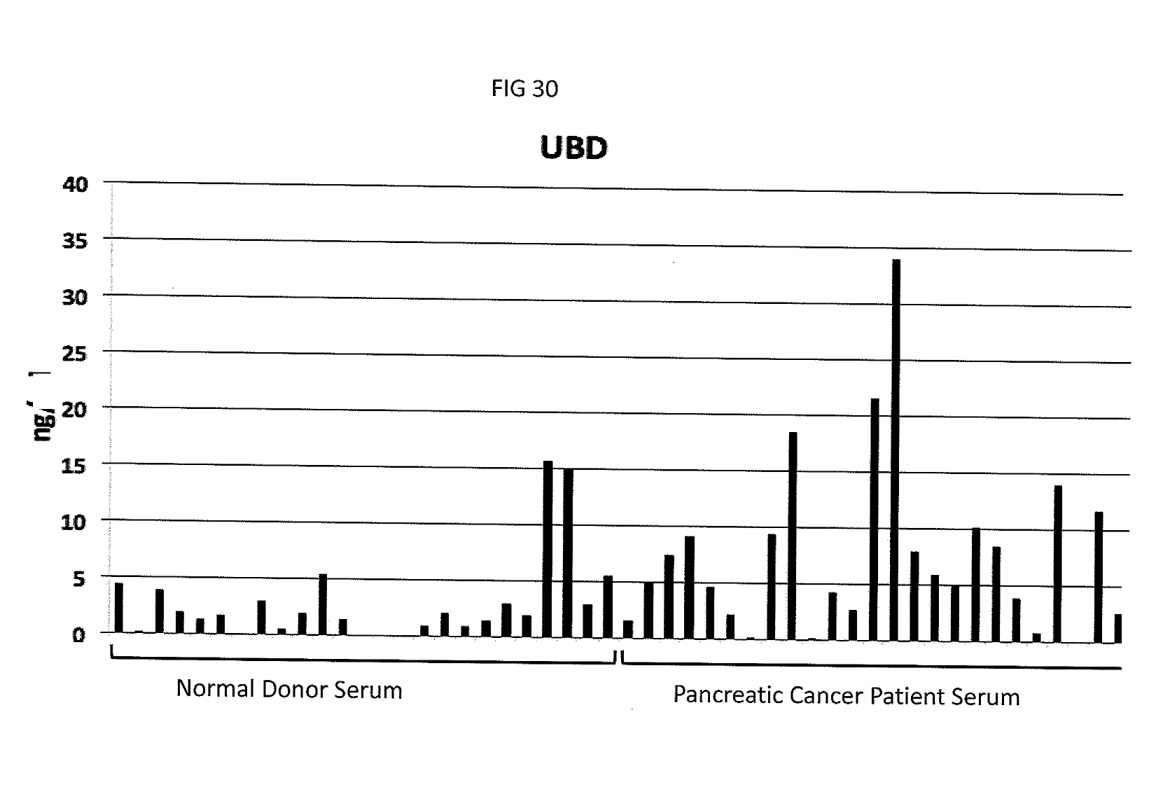

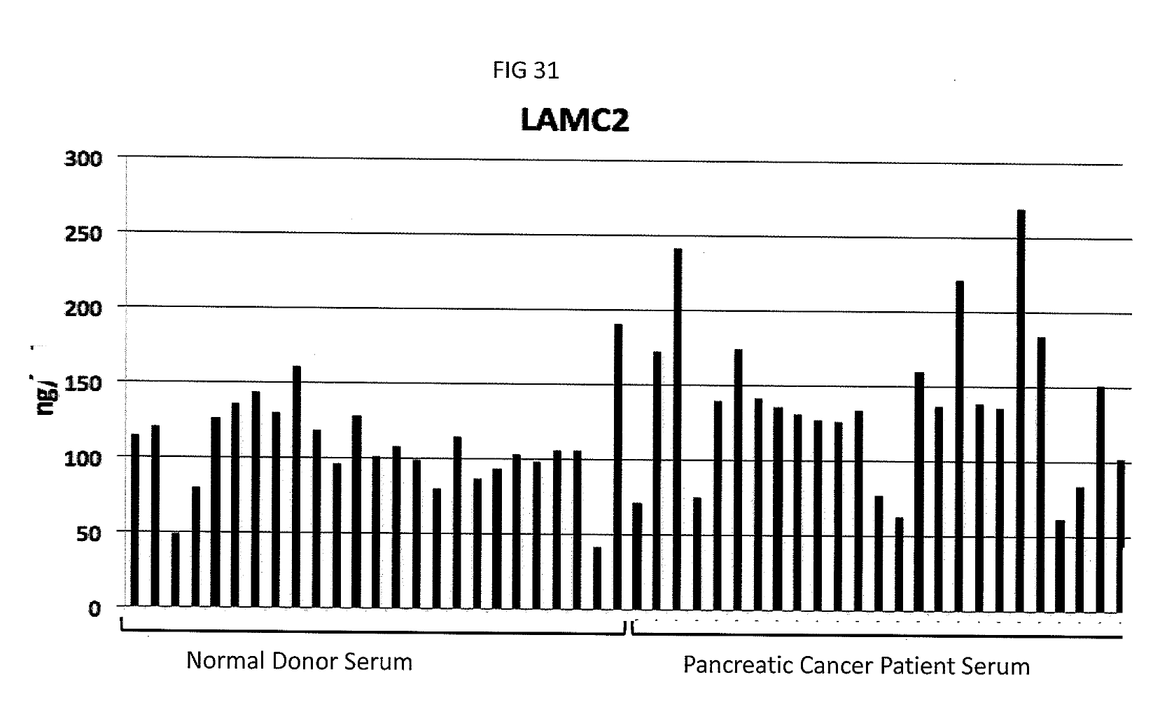

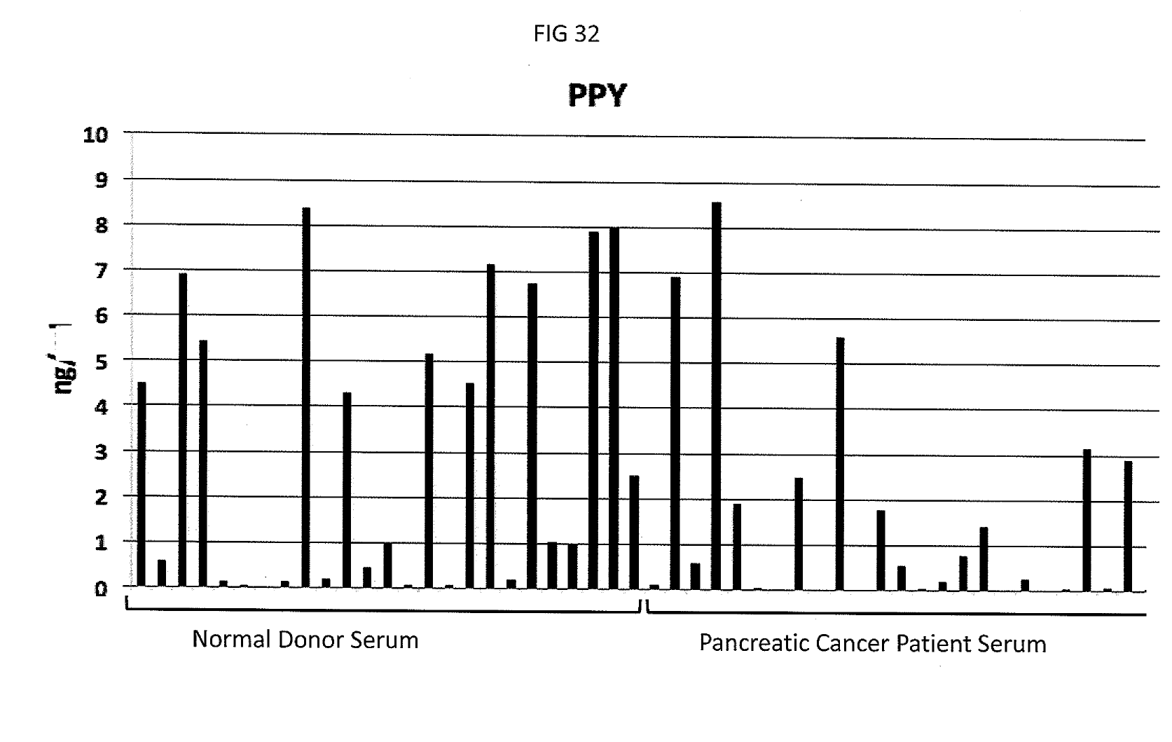

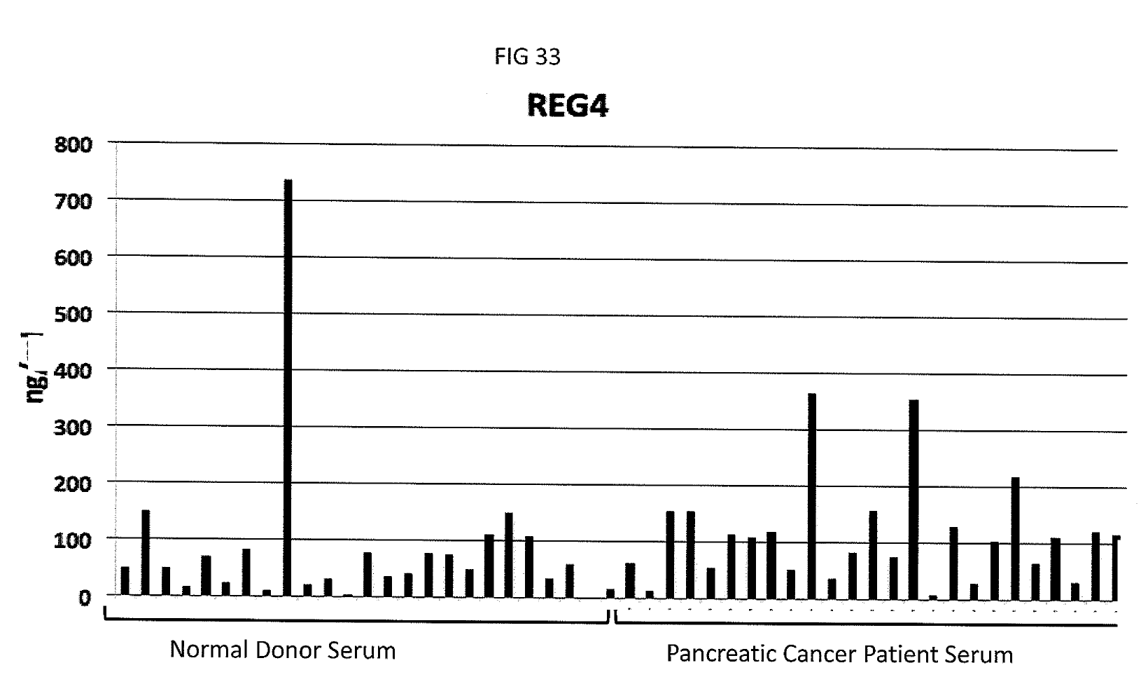

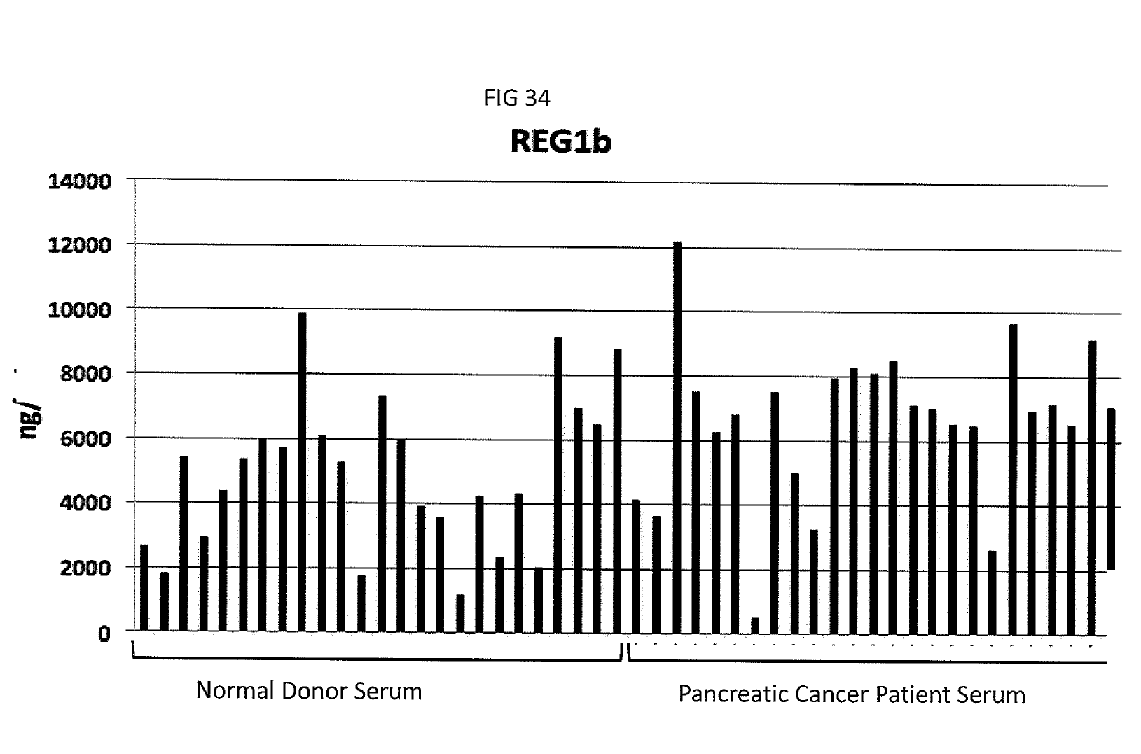

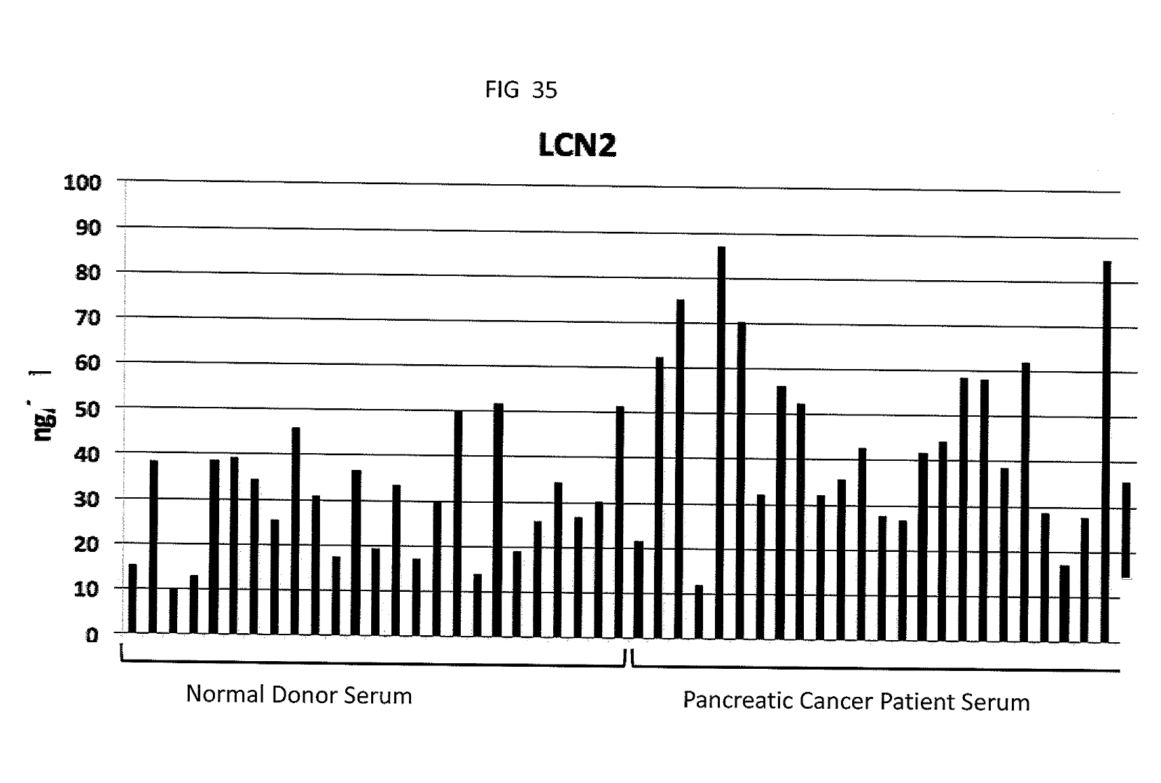

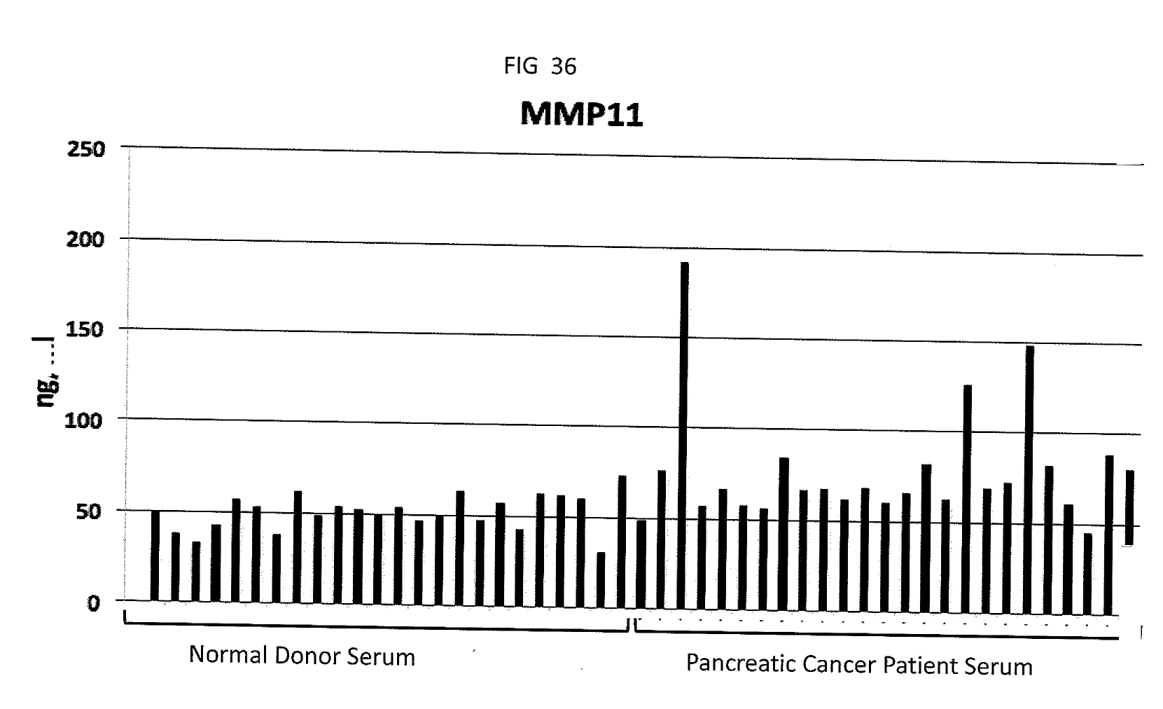

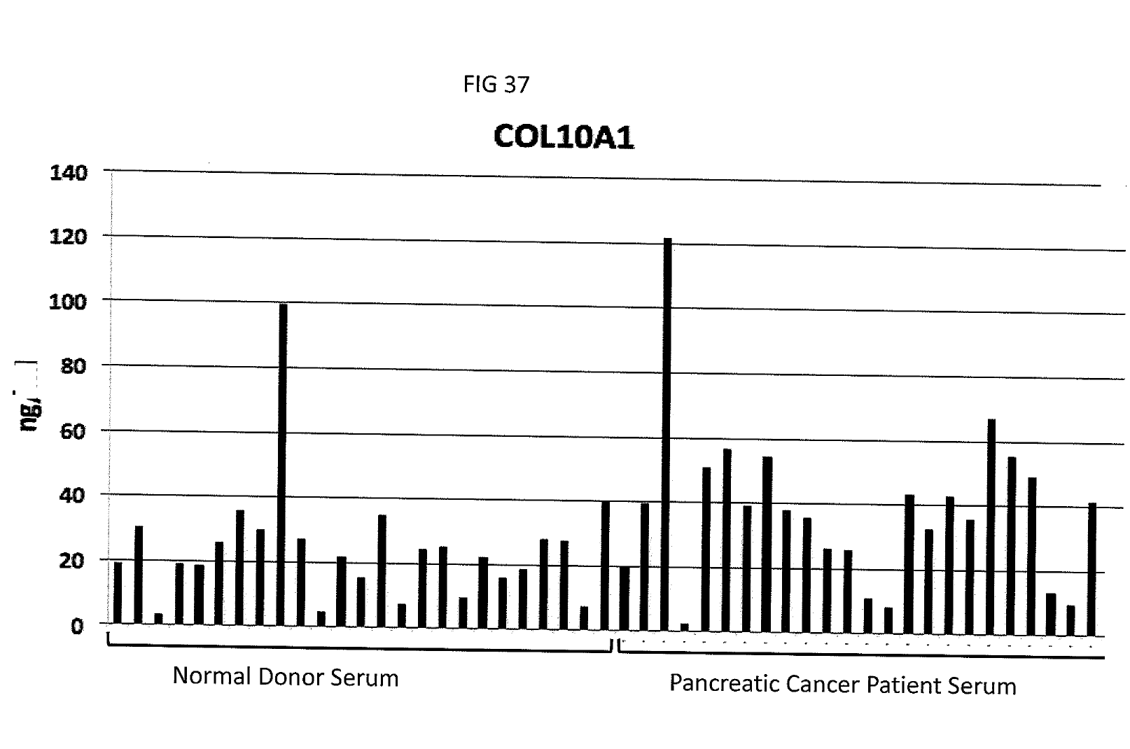

Example 30 provides ELISA data for protein encoded for by the genes: UPD, LAMC2, PPY, REG4, REG1b, LCN2, MMP11, and COL10A (FIGS. 30-37).

Levels of the three protein markers were assayed in serum using a USCN ELISA kit (USCN) according to the manufacturer's instructions. In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution. 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

The results are shown in FIGS. 30-37 that UPD, LAMC2, PPY, REG4, REG1b, LCN2, MMP11, and COL10A are all elevated in pancreatic cancer samples.

### Example 31

Levels of MMP7 protein were assayed in serum using a Luminex kit (Millipore, Bedford, Mass.) according to the manufacturer's instructions. In brief, 200 μL of wash buffer was added per well, shaken for 10 minutes, then decanted. Then, 25 μL of standard or control was added to the appropriate wells and 25 μL of assay buffer was added to the background and sample wells. Then, 25 μL of the appropriate matrix solution was added to the background, standards, and control wells. Then, 25 μL of serum sample was added to the sample wells and then 25 μL of beads were added to each well and incubated overnight at 4° C. with shaking. The contents of the wells was removed and washed 2× with 200 μL wash buffer and then 25 uL detection antibodies was added to each well and incubated for 1 hour at room temperature. Then, 25 μL Streptavidin-Phycoerythrin per well was added and incubated for 30 minutes at room temperature. The contents of the wells were removed and washed 2 times with 200 uL wash buffer and 100 uL of wash buffer was added per well and samples were read on the Luminex 200 instrument (50 μL, 50 beads per bead set). A standard curve was derived from the standards supplied in the kit and the sample values were extrapolated from this curve.

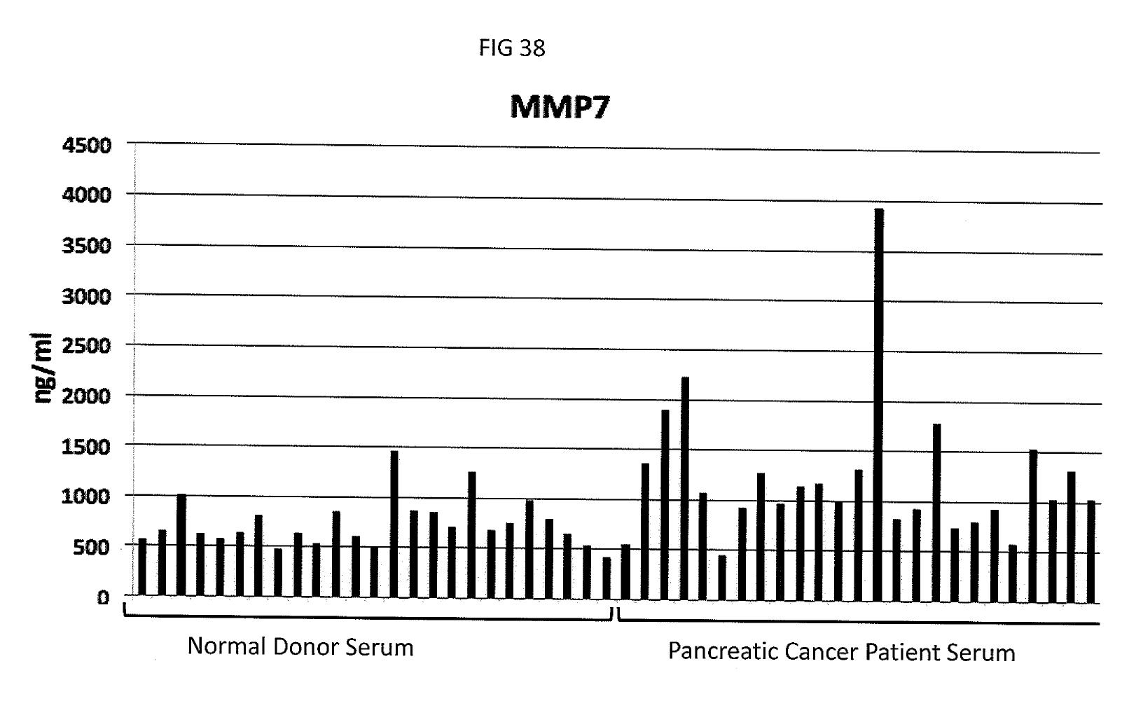

Serum samples from normal subjects, subjects and those with pancreatic, cancer were analyzed. The results, shown in FIG. 38 indicate that protein expression levels of MMP7 in serum of subjects with pancreatic cancer were elevated when compared to normal subjects.

TABLE 1
Genes Associated with Pancreatic Cancer

| | SEQ | | |
|---|---|---|---|
| Symbol | ID NO | Accession | Definition |
| PPY | 1 | NM_0027 [symbol] | Homo sapiens pancreatic polypeptide (PPY), mRNA. |
| CHGB | 2 | NM_0018 [symbol] | Homo sapiens chromogranin B (secretogranin 1) (CHG8), mRNA. |
| ACE2 | 3 | NM_0218 [symbol] | Homo sapiens angiotensin I converting enzyme (peptidyl-dipeptidase A) 2 (ACE2), mRNA. |
| COL10A1 | 4 | NM_0004 [symbol] | Homo sapiens collagen, type X, alpha 1 (COL10A1), mRNA. |
| PRSS7 | 5 | NM_0027 [symbol] | Homo sapiens protease, serine, 7 (enteiokinase) (PRS57), mRNA. |
| MEP1B | 6 | NM_0059 [symbol] | Homo sapiens meprin A, beta (MEP1B), mRNA. |
| MMP12 | 7 | NM_0024 [symbol] | Homo sapiens matrix metallopeptidase 12 (macrophage elastase) (MMP12), mRNA. |
| MMP11 | 8 | NM_0059 [symbol] | Homo sapiens matrix metallopeptidase 11 (stromelysin 3) (MMP11), mRNA. |
| TM4SF20 | 9 | NM_0247 [symbol] | Homo sapiens transmembrane 4 L six family member 20(TM4SF20), mRNA. |
| AQP10 | 10 | NM_0804 [symbol] | Homo sapiens aquaporin 10 (AQP10), mRNA. |
| REG1P | 11 | NR_00271 [symbol] | Homo sapiens regenerating islet-derived 1 pseudogene (REG1P), non-coding RNA. |
| KCNIP1 | 12 | NM_0145 [symbol] | Homo sapiens Kv channel interacting protein 1 (KCNIP1), transcript variant 2, mRNA. |
| UBD | 13 | NM_0063 [symbol] | Homo sapiens ubiquitin D (UBD), mRNA. |
| MS4A10 | 14 | NM_2068 [symbol] | Homo sapiens membrane-spanning 4-domains, subfamily A, member 10 (MS4A10), mRNA. |
| CXCL5 | 15 | NM_0029 [symbol] | Homo sapiens chemokine (C-X-C motif) ligand 5 (CXCL5), mRNA. |
| REG3G | 16 | NM_0010 [symbol] | Homo sapiens regenerating islet-derived 3 gamma (REG3G), transcript variant 1, mRNA. |
| PTPRR | 17 | NM_0028 [symbol] | Homo sapiens protein tyrosine phosphatase, receptor type, R (PTPRR), transcript variant 1, mRNA. |
| C6orf222 | 18 | NM_0010 [symbol] | Homo sapiens chromosome 6 open reading frame 222 (C6orf222), mRNA. |
| RBP2 | 19 | NM_0041 [symbol] | Homo sapiens retinol binding protein 2, cellular (RBP2), mRNA. |
| TNFRSF6B | 20 | NM_0038 [symbol] | Homo sapiens tumor necrosis factor receptor superfamily, member 6b, decoy (TNFRSF6B), |
| | | | transcript variant M68E, mRNA. |
| TNFRSF6B | 21 | NM_0329 [symbol] | Homo sapiens tumor necrosis factor receptor superfamily, member 6b, decoy (TNFRSF6B), |
| | | | transcript variant M68C, mRNA. |
| CST1 | 22 | NM_0018 [symbol] | Homo sapiens cystatin SN (CST1), mRNA. |
| CTSE | 23 | NM_0019 [symbol] | Homo sapiens cathepsin E (CTSE), transcript variant 1, mRNA. |
| SI | 24 | NM_0010 [symbol] | Homo sapiens sucrase-isomaltase (alpha-glucosidase) (SI), mRNA. |
| LUM | 25 | NM_0023 [symbol] | Homo sapiens lumican (LUM), mRNA. |
| C5orf46 | 26 | NM_2069 [symbol] | Homo sapiens chromosome 5 open reading frame 46 (C5orf46), mRNA. |
| MMP7 | 27 | NM_0024 [symbol] | Homo sapiens matrix metallopeptidase 7 (matrilysin, uterine) (MMP7), mRNA. |
| GABRP | 28 | NM_0142 [symbol] | Homo sapiens gamma-aminobutyric acid (GABA) A receptor, pI (GABRP), mRNA. |
| COMP | 29 | NM_0000 [symbol] | Homo sapiens cartilage oligomeric matrix protein (COMP), mRNA. |
| FNDC1 | 30 | NM_0325 [symbol] | Homo sapiens fibronectin type III domain containing 1 (FNDC1), mRNA. |

[symbol]

indicates data missing or illegible when filed

TABLE 2
Genes Associated with Pancreatic Cancer

| | SEQ | | |
|---|---|---|---|
| Symbol | ID NO | Accession | Definition |
| WNT4 | 31 | NM_030761.3 | Homo sapiens wingless-type MMTV integration site family, member 4 (WNT4), mRNA. |
| PCSK2 | 32 | NM_002594.2 | Homo sapiens proprotein convertase subtilisin/kexin type 2 (PCSK2), mRNA. |
| CHGB | 33 | NM_001819.1 | Homo sapiens chromogranin B (secretogranin 1) (CHGB), mRNA. |
| C19orf30 | 34 | NM_174947.2 | Homo sapiens chromosome 19 open reading frame 30 (C19orf30), mRNA. |
| PCSK1 | 35 | NM_000439.3 | Homo sapiens proprotein convertase subtilisin/kexin type 1 (PCSK1), mRNA. |
| PCSK1N | 36 | NM_013271.2 | Homo sapiens proprotein convertase subtilisin/kexin type 1 inhibitor (PCSK1M), mRNA. |
| PTPRN | 37 | NM_002845.2 | Homo sapiens protein tyrosine phosphatase, receptor type, N (PTPRN), mRNA. |
| SCGN | 38 | NM_006998.3 | Homo sapiens secretagogin, EF-hand calcium binding protein (SCGN), mRNA, |
| KCHK16 | 39 | NM_032115.2 | Homo sapiens potassium channel, subfamily K, member 16 (KCNK16), mRNA. |
| KCNMB2 | 40 | NM_005832.3 | Homo sapiens potassium large conductance calcium-activated channel, subfamily M, |
| | | | beta member 2 (KCNMB2) |
| PPY | 41 | NM_002722.3 | Homo sapiens pancreatic polypeptide (PPY), mRNA. |
| SMOC1 | 42 | NM_001034852.1 | Homo sapiens SPARC related modular calcium binding 1 (SMOC1), transcript variant 1, mRNA. |
| CFC1 | 43 | NM_032545.2 | Homo sapiens cripto, FRL-1, cryptic family 1 (CFC1), mRNA. |
| ABCC8 | 44 | NM_000352.3 | Homo sapiens ATP- binding cassette, sub-family C (CFTR/MRP), member 8 (ABCC8), mRNA. |
| CA849062 | 45 | CA849062 | Ir69h03.y1 HR85 islet Homo sapiens cDNA clone IMAGE: 6607902 5, mRNA sequence |
| DRD1IP | 46 | NM_015722.2 | Homo sapiens dopamine receptor D1 interacting protein (DRD1IP), mRNA. |
| CALY | 47 | NM_015722.3 | Homo sapiens calcyon neuron-specific vesicular protein (CALY), mRNA. |
| CNTN1 | 48 | NM_001843.2 | Homo sapiens contactin 1 (CNTN1), transcript variant 1, mRNA. |
| GAD2 | 49 | NM_000818.1 | Homo sapiens glutamate decarboxylase 2 (pancreatic islets and brain, 65kDa) (GAD2), mRNA. |
| LOC64246 | 50 | XR_016169.1 | PREDICTED: Homo sapiens similar to ankyrin repeat domain 30A (LOC642460), mRNA. |
| LOC65013 | 51 | NM_001080841.1 | Homo sapiens seven transmembrane helix receptor (LOC650137), mRNA. |
| VIP | 52 | NM_003381.2 | Homo sapiens vasoactive intestinal peptide (VIP), transcript variant 1, mRNA. |

TABLE 4
Probe Sequences for Genes Associated with Pancreatic Cancer

| | SEQ ID | | | |
|---|---|---|---|---|
| Symbol | NO. | Accession | Probd_Id | Probe_Sequence |
| PPY | 1 | NM_002722.3 | ILMN_1751445 | GGACTTATAATGCCACCTTCTGTCTCCTACGACTCCATGAGCAGCGCCAG |
| CHGB | 2 | NM_001819.1 | ILMN_1765966 | TCAGCCAAAGGGGCTGACTGTCATTGGAGCGGTGGGCACTGTTAAGAAGC |
| ACE2 | 3 | NM_021804.1 | ILMN_1667018 | ACATCTCCCTGACAAGACAAAACTAGAGCCAGGGGCCTCCGTGAACTCCC |
| COL10A1 | 4 | NM_000493.3 | ILMN_1672776 | CCCCTAAAATATTTCTGATGGTGCACTACTCTGAGGCCTGTATGGCCCCT |
| PRSS7 | 5 | NM_002772.1 | ILMN_1695969 | GCCTAATCGCCCCGGAGTGTATGCCAGGGTCTCAAGGTTTACCGAATGGA |
| MEP1B | 6 | NM_005925.2 | ILMN_1734694 | GGTGGTACATGGGAGAAAGGTGTGAAAAGAGAGGCTCCACCCGAGACACC |
| MMP12 | 7 | NM_002426.2 | ILMN_2073758 | TCTATTTGAAGCATGCTCTGTAAGTTGCTTCCTAACATCCTTGGACTGAG |
| MMP11 | 8 | NM_005940.3 | ILMN_1655915 | CAGGTCTTGGTAGGTGCCTGCATCTGTCTGCCTTCTGGCTGACAATCCTG |
| TM4SF20 | 9 | NM_024795.2 | ILMN_1775830 | GACTGCAACTCATCACATGAGGTCAGGTATGGAATTTTCCACTTGTGGTG |
| AQP10 | 10 | NM_080429.2 | ILMN_2090004 | CCCTGCAGAGGATGCTCGTTTTGCAGAGAAGGCAGTGTTCCTCTATTCCC |
| REG1P | 11 | NR_002714.1 | ILMN_1785302 | CTATTTATCTCTGAGGATCGACCGGGGACTGGGATAGAGGAAGGGTGACC |
| KCNIP1 | 12 | NM_014592.2 | ILMN_2368856 | GAGGTCTCTCCAGCTGTTTCAAAATGTCATGTAACTGGTGACACTCAGCC |
| UBD | 13 | NM_006398.2 | ILMN_1678841 | CCTCCTCCAGGTGCGAAGGTCCAGCTCAGTGGCACAAGTGAAAGCAATGA |
| MS4A10 | 14 | NM_206893.2 | ILMN_1794921 | GGCTAGCTCTGCCAATCACTTACTGTGCGGGTTTGACTCAGTCCCTTCCC |
| CXCL5 | 15 | NM_002994.3 | ILMN_1752562 | GCAAGTTCCCTCCCCACTCACAGCTTTGGCCCCTTTCACAGAGTAGAACC |
| REG3G | 16 | NM_001008387.1 | ILMN_1676795 | CTGTGTGTCCTCCCGCTGACCACACTTCCTTTAGTGACCCGATTGCCTCC |
| PTPRR | 17 | NM_002849.2_ | ILMN_1679051 | GGTGGCTTCTGCTCCAGAACTCTATCCACTGTATTTCCACATCGTGAGTC |
| C6orf222 | 18 | NM_001010903.3 | ILMN_1671087 | CATGGGGGAAAGGGTGTTAAACCTGGATGGCAAGTGAGATCTTGTGACCC |
| RBP2 | 19 | NM_004164.2 | ILMN_2219681 | AGAAGGAGAACCGCGGCTGGAAGCAGTGGATTGAGGGGGACAAGCTGTAC |
| TNFRSF6B | 20 | NM_003823.2 | ILMN_1661825 | AAGGAGGTGGCATGTCGGTCAGGCACAGCAGGGTCCTGTGTCCGCGCTGA |
| TNFRSF6B | 21 | NM_032945.2 | ILMN_2331232 | TGCCGCACCGGCTTCTTCGCGCACGCTGGTTTCTGCTTGGAGCACGCATC |
| CST1 | 22 | NM_001898.2 | ILMN_1753449 | ATCCAGGTGTCAAGAATCCTAGGGATCTGTGCCAGGCCATTCGCACCAGC |
| CTSE | 23 | NM_001910.2 | ILMN_1799887 | TGCTGGTGCCTCTCTATTGGTAATGTTAAGACTGCTGGGTGGGTTTGGAG |
| SI | 24 | NM_001041.1 | ILMN_1739144 | GCTTGGATCCCTTCATGTATGGGGGAAAGGAACTACTCCTGTCAATGCAG |
| LUM | 25 | NM_002345.3 | ILMN_2167805 | CTGTCAAATGATGTGCAAAACCTTTTACTGGTTGCATGGAAATCAGCCAA |
| C5orf46 | 26 | NM_206966.2 | ILMN_1718866 | GGCAAAGACCCAAAGCCAGACTTCCCCAAATTCCTAAGCCTCCTGGGCAC |
| MMP7 | 27 | NM_002423.3 | ILMN_1685403 | GCTCACTTCGATGAGGATGAACGCTGGACGGATGGTAGCAGTCTAGGGAT |
| GABRP | 28 | NM_014211.1 | ILMN_1689146 | TTGCGGGTGAGCTGCCCTCTCCAAATCCAGCCAGAGATGCACATTCCTCG |
| COMP | 29 | NM_000095.2 | ILMN_1677636 | AGAGGACTATGAGACCCATCAGCTGCGGCAAGCCTAGGGACCAGGGTGAG |
| FNDC1 | 30 | NM_032532.1 | ILMN_2163873 | TCCAGGAATAGCATATGCACGCTGTTCTTGCTTCATGGAATGCTACATGC |

TABLE 5
Probe Sequences Associated With Pancreatic Cancer

| | SEQ ID | | | |
|---|---|---|---|---|
| Symbol | No. | Accession | Probe_Id | Probe_Sequence |
| WNT4 | 32 | NM_030761.3 | ILMN_1666392 | CCTGCGCCGGCAACCACCTAGTGGCCCAGGGAAGGCCGATAATTTAAACA |
| PCSK2 | 32 | NM_002594.2 | ILMN_1695003 | GGCCAGTGGAAATTCAGGTGAAAATGTTCATCAATTCCCATTGCATCACC |
| CHGB | 33 | NM_001819.1 | ILMN_1765966 | TCAGCCAAAGGGGCTGACTGTCATTGGAGCGGTGGGCACTGTTAAGAAGC |
| C19orf30 | 34 | NM_174947.2 | ILMN_1730818 | GCTCCCCACAACTGACCCTGCCTTCTTCTGCAAGCTCCATTTCATCAAGG |
| PCSK1 | 35 | NM_000439.3 | ILMN_1710859 | GTAGAGGGTGTTTGCAGAGCAATGCCCGTAATGCTTAGAGAATGTTCTCC |
| PCSK1N | 36 | NM_013271.2 | ILMN_1755582 | AGCTGTTGAGGTACTTGCTGGGACGGATTCTTGCGGGAAGCGCGGACTCC |
| PTPRN | 37 | NM_002846.2 | ILMN_1658576 | CTGGGAGTTCCCTGAACATCTGTGTGTGTCCCCCTATGCTCCAGTATGGA |
| SCGN | 38 | NM_006998.3 | ILMN_1789648 | CTCCCAAAGACTCAGCTCCCCTGTTAGATGGCTCTGCCTGTCCTTCCCCA |
| KCNK16 | 39 | NM_032115.2 | ILMN_1792186 | AAGGACAGAGGTGTGGGAGACCCAAGGTGGTCTTGAGATTGACAGACAGC |
| KCNMB2 | 40 | NM_005832.3 | ILMN_1687331 | AACTGAGAGAAAGAGCAACAAAGCGGCGAGTGGTGTGAGAGGGCAGCACG |
| PPY | 41 | NM_002722.3 | ILMN_1751445 | CGACTTATAATGCCACCTTCTGTCTCCTACGACTCCATGAGCAGCGCCAG |
| SMOC1 | 42 | NM_001034852.1 | ILMN_1791202 | CAACAGGGAAACTCTTCACCTCCCTGCAAACCTACCAGTGAGGCTCCCAG |
| CFC1 | 43 | NM_032545.2 | ILMN_1668099 | GCACTAAGAGAAGGAGACTCTCAAACCAAAAATGACCTGGAGGCACCATG |
| ABCC8 | 44 | NM_000352.3 | ILMN_1767129 | TTCGTCCGTGCAGACAAGTGACCTGCCAGAGCCCAAGTGCCATCCCACAT |
| CA849062 | 45 | CA849062 | ILMN_1915987 | TGTCCTCGCTATGAGATTTCCCACCACCCACGCTGGTGTTCACATCTAGC |
| DRD1IP | 46 | NM_015722.2 | ILMN_1771622 | CCCCCGTGGGTCTCTGACTCTGTCGCTTTTCTCTAAGTAAAGATTTCACG |
| CALY | 47 | NM_015722.3 | ILMN_3238986 | AAGATCTGCACGCCGCTGACCCTGGAGATGTACTACACGGAGATGGACCC |
| CNTN1 | 48 | NM_001843.2 | ILMN_1661852 | CTCAAGTCAAAATTTCAGGTGCACCCACCCTATCCCCAAGTCTTCTCGGC |
| GAD2 | 49 | NM_000818.1 | ILMN_1728301 | CAGCTGCCAAAATCGTAGGTGTTGGCTCTGCTGGTCACTGGAGTAGTTGC |
| LOC64246 | 50 | XR_016169.1 | ILMN_1672000 | AAGCCTACCTGTGGAAGGAAAGTTTCTCTTCCAAATAAAGCCTTAGAATT |
| LOC65013 | 51 | NM_001080841.1 | ILMN_2103003 | TAGTGGCCTGATGACACTCATGTGCTTTCTGGGACTTCTGGCCTCCTATG |
| VIP | 52 | NM_0033812.2 | ILMN_2357543 | CTTCGGCATGGCCTCTTTACAGGGCACCTTCTGCTCTCAGGTTGGGTGAC |

TABLE 6
Genes Associated With Pancreatic Cancer

| Symbol | Accession | Definition |
|---|---|---|
| LAMC2 | NM_005562.1 | Homo sapiens laminin, gamma 2 (LAMC2), transcript variant 1, mRNA. |
| LCN2 | NM_005564.3 | Homo sapiens lipocalin 2 (LCN2), mRNA. |
| REG1B | NM_006507.2 | Homo sapiens regenerating islet-derived 1 beta (pancreatic stone protein, pancreatic thread protein) (REG1B), mRNA. |
| REG4 | NM_032044.2 | Homo sapiens regenerating islet-derived family member 4 (REG4), mRNA. |

TABLE 7
Probe Sequences Associated With Pancreatic Cancer

| | | SEQ ID | | |
|---|---|---|---|---|
| Symbol | Accession | NO: | Probe_Id | Probe_Sequence |
| LAMC2 | NM_005562.1 | 53 | ILMN_16538 [symbol] | CGCTGGGGTTACAATCCAAGACACACTCAACACATTAGACGG |
| | | | | CCTCCTGC |
| LCN2 | NM_005564.3 | 54 | ILMN_16922 [symbol] | CCACATCGTCUCCCTGTCCCAATCGACCAGTGTATCGACGGC |
| | | | | TGAGTGC |
| REG1B | NM_006507.2 | 55 | ILMN_16814 [symbol] | TGCATCGCTGATCHCAGTACCTTCACCTUCTCAGTCTUAGAG |
| | | | | CCCTG |
| REG4 | NM_032044.2 | 56 | ILMN_21693 [symbol] | GGTCTGGCAAGTCCATGGGTGGGAACAAGCACTGTGCTGAGA |
| | | | | TGAGCTCC |

[symbol]

indicates data missing or illegible when filed

1. A method of detecting pancreatic cancer in a subject comprising a) obtaining a sample from a subject b) contacting the sample obtained from the subject with one or more agents that detect expression of a panel of markers encoded by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof; c) contacting a non-cancerous cell, with the one or more agents from b); and d) comparing the expression level of the panel of markers encoded for by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2, or a complement thereof in the sample obtained from the subject with the expression level of the panel of markers encoded for by the genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2 or a complement thereof, in the non-cancerous cell, wherein a higher level of expression of the panel of markers encoded for by genes PPY, CHGB, ACE2, COL10A1, PRSS7, MEP1B, MMP12, MMP11, TM4SF20, AQP10, REG1P, KCNIP1, UBD, MS4A10, CXCL5, REG3G, PTPRR, C6orf222, RBP2, TNFRSF6B, TNFRSF6B, CST1, CTSE, SI, LUM, C5orf46, MMP7, GABRP, COMP, FNDC1, WNT4, PCSK2, CHGB, C19orf30, PCSK1, PCSK1N, PTPRN, SCGN, KCNK16, KCNMB2, PPY, SMOC1, CFC1, ABCC8, CA849062, DRD1IP, CALY, CNTN1, GAD2, LOC642460, LOC650137, VIP, LAMC2, REG4, REG1b, LCN2 or a complement thereof in the sample compared to the non-cancerous cell indicates that the subject has pancreatic cancer.

2. The method of claim 1, wherein the subject is a human.

3. The method of claim 1, wherein the sample is a bodily fluid.

4. The method of claim 3, wherein the bodily fluid is blood.

5. The method of claim 3, wherein the bodily fluid is serum.

6. The method of claim 3, wherein the bodily fluid is urine.

7. The method of claim 1, wherein the sample is a tissue sample.

8. The method of claim 1, wherein the sample is comprised of cells.

9. The method of claim 1, wherein the one or more agents is a nucleic acid.

10. The method of claim 1, wherein the one or more agents is a protein.

11. The method of claim 10, wherein the protein is an antibody.

12. A kit for detecting pancreatic cancer in sample comprising one or more agents of claim 1.

13. The kit of claim 12, wherein the one or more agents is a nucleic acid.

14. The kit of claim 12, wherein the one or more agents is a protein.

15. The kit of claim 12, wherein the protein is an antibody.

16. The kit of claim 12, further comprising one or more controls.

17. The kit of claim 16 wherein the control is a positive control.

18. The kit of claim 17 wherein the positive control comprises pancreatic cancer cells.

19. The kit of claim 16, wherein the one or more controls is a negative control.

20. The kit of claim 19, wherein the negative control comprises non-cancerous pancreatic cells.
